(19) United States
(12) Patent Application Publication
(10) Pub. No.: US 2026/0014395 A1
(43) Pub. Date: Jan. 15, 2026

(54) WEARABLE AND AUTOMATED ULTRASOUND THERAPY DEVICES AND METHODS

(71) Applicant: Cortery AB, Stockholm (SE)

(72) Inventors: Filip Ludwig PETERS, Domsten (SE); STOKELY Christopher Lee, Houston, TX (US); Mena NADUM, Göteborg (SE); Ana Candela CELDRAN, Upplands Väsby (SE); Lars Gunnar Henrik STAAF, Lindome (SE); Mohammad Yusuf MULLA, Norrköping (SE); Christer JOHANSSON, Göteborg (SE)

(21) Appl. No.: 18/993,575

(22) PCT Filed: Jul. 12, 2023
(86) PCT No.: PCT/EP2023/069401

§ 371 Date: Jan. 12, 2025

Related U.S. Application Data
(63) Continuation-in-part of application No. PCT/EP2022/069521, filed on Jul. 12, 2022.
Application No. PCT/EP2022/069521 is a continuation-in-part of application No. 18/155,063, filed on Jan. 16, 2023, now Pat. No. 12,053,652.
Application No. 18/155,063 is a continuation-in-part of application No. PCT/EP2022/069521, filed on Jul. 12, 2022.
(60) Provisional application No. 63/479,544, filed on Jan. 11, 2023.
(60) Provisional application No. 63/480,000, filed on Jan. 14, 2023.

(30) Foreign Application Priority Data
Jul. 12, 2022 (WO) PCT/EP2022/069521

(51) Int. Cl. A61N 7/00

(52) U.S. Cl. CPC A61N 7/00; A61N 2007/0052

(57) ABSTRACT

Described are devices and methods for administering targeted ultrasound therapy. The device consists of a wearable housing designed to conform to a part of the user's body, with an array of ultrasound transducer units on the skin-facing side. These transducers can generate ultrasound therapy to a target therapy area of the user, with frequency, intensity, treatment area, and duration parameters optimized for a therapeutic application. Incorporated within the device is a communication system, an energy source, and a processor. The processor's role is to execute instructions, including assessing the user's health status based on received data, processing and analyzing ultrasound data to gauge transmission quality, determining if the current transmission quality is within thresholds, initiating ultrasound therapy when the conditions are right, and adjusting ultrasound therapy parameters real-time or near real-time if needed during treatment. The real-time or near real-time adjustment is made through various parameters like pulsing frequency, pulse train frequency, the number of treated spots, duration, and treatment zone area. This device provides a personalized, adaptive approach to ultrasound therapy, optimizing the treatment process for each individual user.

### PRIORITY

This application claims benefit of and priority to U.S. Non-Provisional patent application Ser. No. 18/155,063 filed on Jan. 16, 2023, U.S. Provisional Patent Application No. 63/479,544 filed on Jan. 11, 2023, U.S. Provisional Patent Application No. 63/480,000 filed on Jan. 14, 2023, and PCT application number PCT/EP2022/069521 filed on Jul. 12, 2022.

### BACKGROUND

### Technical Field

The specification is generally directed toward wearable and/or automated ultrasound therapy devices. More particularly, but not limited to, a wearable and automated ultrasound therapy device for treating and preventing one or more of kidney disease, cardiovascular disease, neurodegenerative disease, peripheral artery disease, deep vein thrombosis and inducing pulmonary rehabilitative effects.

### Description of the Related Art

Typically, the human body's kidneys function to filter out extra fluid and certain ions. Renovascular disease patients have substantially impaired kidney function due to one or more of chronic renal disease (CRD), diabetic kidney disease and acute kidney injury may be characterized by a gradual decline in renal function over months or years. Angio-genetic, anti-inflammatory and/or regenerative therapy can be important goals of treatment in these diseases.

Further, pulmonary rehabilitation (PR) enables people with lung problems to breathe and live more comfortably. With lung conditions like chronic obstructive pulmonary disease (COPD) or Idiopathic Pulmonary Fibrosis (IPF), a patient could need pulmonary rehabilitation with the goal of inducing anti-inflammatory and/or muscle relaxing effects or inducing angiogenesis and blood circulation to aid the body in breaking down fibrous tissue.

Further, deep vein thrombosis (DVT) occurs when a blood clot (thrombus) forms in one or more of the deep veins in the body, usually in the legs which can cause leg pain or swelling. Reducing swelling and inducing anti-thrombotic effects is an important goal of therapy.

Furthermore, a common disorder called peripheral arterial disease causes arteries to constrict, reducing blood flow to the arms or legs. In peripheral artery disease (PAD), the blood supply to the legs or arms is insufficient to meet demand. Other symptoms, such as claudication, may result from this. Typically, peripheral artery disease indicates an accumulation of fatty deposits in the arteries (atherosclerosis). Vascular narrowing brought on by atherosclerosis can lessen blood flow to the arms and legs. Revascularization can be an appropriate therapeutic goal for PAD.

Low intensity ultrasound treatment methods have been used in treating soft tissue injuries or damage and reducing fatty deposits commonly known as cellulite.

Some devices generate low-intensity ultrasound. US patent US10058340B2 issued to Iulian Cioanta et al. discloses an ultrasound applicator that includes housing multiple reflectors with ultrasound generators projecting into a shared cavity. The multiple ultrasound generators can produce a variety of ultrasound focal volumes and wavefronts for medical treatment. Further, US patent application US20200206072A1 published by Christopher Capelli et al. discloses a method for acoustic treatment of tissue to disperse vacuoles within the tissue. The method includes a step of directing pulsed acoustic waves from the acoustic wave generator into the tissue containing the vacuoles. Further, the method of Christopher Capelli et al. includes a step of identifying the location of tissue containing vacuoles, and/or coupling (e.g., acoustically) an acoustic wave generator to the tissue containing the vacuoles.

### SUMMARY

The above devices are intended to be utilized by a trained, and skilled medical professional who can apply the treatment to a patient in a clinical setting. Furthermore, the existing devices are costly and require specific knowledge about human anatomy, treatment protocol, regimen, pressure to be applied by the device, duration, and frequency of treatment. Further, these devices cannot track the health status of the user over time.

To the Applicant's knowledge, no wearable ultrasound therapy devices exist that utilize ultrasound for automatically treating kidney disease, inducing pulmonary rehabilitative effects, treating peripheral artery disease, and/or treating deep vein thrombosis.

This specification recognizes that there is a need for a portable, cost-effective, and efficient wearable and automated ultrasound therapy device to improve and measure pulmonary function, renal function, peripheral arterial function, and deep vein thrombosis (DVT) symptoms. Further, there is a need for a device that does not require a medical professional and/or assistant to have any specific abilities or training for ultrasound-based treatment of kidney disease, inducing pulmonary rehabilitative effects, peripheral artery disease, and deep vein thrombosis. Furthermore, there is a need for a wearable and automated ultrasound therapy device that can reduce the requirement for hospital-based visits and reduce the amount of resources needed by the hospital to treat a patient.

In some embodiments, a wearable and automated ultrasound therapy device for treating kidney disease, inducing pulmonary rehabilitative effects, treating peripheral artery disease, and treating deep vein thrombosis is provided.

An aspect relates to a device for generating ultrasound treatment to a target therapy area of a user. The device comprises a wearable housing including a skin-facing side; an array of ultrasound transducer units on the skin-facing side; an energy source; and a processor for processing instructions. The instructions comprise: loading transducer parameters for a therapeutic application, the transducer parameters containing at least one of frequency, intensity, treatment zone area, and duration; triggering the array of ultrasound transducer units to emit test waves according to a transmission quality test routine; determining if a backscatter or a reflection of the test waves passes the transmission quality test; initiating an ultrasound therapy treatment process when the current conditions pass the transmission quality test. The ultrasound treatment process comprises: generating an ultrasound therapy by the array of ultrasound transducer units.

The transmission quality test routine is configured to assess the overall quality of ultrasound therapy transmission. The routine provides specific instructions and examples that enable a skilled person within the art to perform the test with accuracy and reliability. The quality approval test routine comprises the following steps:

Manual Assessment: One approach to assessing the quality of ultrasound therapy transmission is through manual evaluation. A skilled operator visually inspects the raw ultrasound data to determine if it meets one or more preset thresholds for acceptable quality. The operator compares the observed data with known criteria or reference standards to make an informed judgment on the quality of the transmission.

Threshold-based Assessment: An alternative approach involves setting predefined thresholds for specific parameters or features of the ultrasound data. The data is then compared against these thresholds to determine if it meets the required quality criteria. For example, thresholds can be defined for signal-to-noise ratio, spatial resolution, or image clarity. If the data surpasses the thresholds, it is considered to have met the quality requirements for generating ultrasound therapy.

Machine Learning Analysis: In addition to manual or threshold-based assessments, machine learning models can be utilized to analyze the raw ultrasound data and estimate its quality. The machine learning models can be trained on a dataset comprising ultrasound data, either raw or processed, along with corresponding quality labels. The labels indicate whether the data is of sufficient quality for generating ultrasound therapy. The machine learning models can be supervised, where the training data includes labeled examples, or unsupervised, where the models learn patterns and relationships within the data without explicit labels. The trained machine learning models analyze the relevant features of the ultrasound data and generate a quality estimation or classification. The estimation indicates the likelihood that the ultrasound transmission meets the predefined quality thresholds. This automated approach provides a fast and objective assessment of the ultrasound data, enhancing the efficiency and accuracy of the quality approval process.

It should be noted that the specific machine learning algorithms and techniques used for analysis may vary depending on the implementation. Examples include support vector machines, decision trees, neural networks, or clustering algorithms. The training of the machine learning models may involve preprocessing steps, feature extraction, and feature selection to optimize the model's performance. In addition to the manual assessment, threshold-based assessment, and machine learning analysis described earlier, there are several other approaches that can be used for quality assessment of ultrasound therapy. These approaches include:

Statistical Analysis: Statistical methods can be employed to analyze various statistical properties of the ultrasound data. This can involve measures such as mean, standard deviation, variance, or correlation coefficients. By comparing these statistical properties against predefined benchmarks or reference values, the quality of the ultrasound transmission can be assessed.

Signal Processing Techniques: Signal processing techniques can be utilized to enhance the ultrasound data and extract relevant information. These techniques may involve noise reduction, filtering, image enhancement, or feature extraction algorithms. The processed data can then be evaluated based on specific criteria to determine its quality for generating ultrasound therapy.

Quantitative Metrics: Quantitative metrics can be defined to measure specific aspects of the ultrasound data, such as image resolution, contrast, or spatial uniformity. These metrics can be calculated based on mathematical formulas or algorithms that quantify the desired attributes. By comparing the calculated metrics against predefined thresholds, the quality of the ultrasound transmission can be evaluated.

Reference Standards: Reference standards or guidelines can be established by regulatory bodies or industry organizations. These standards define specific criteria and requirements for acceptable ultrasound data quality. The ultrasound transmission can be assessed against these reference standards to ensure compliance and quality assurance.

In summary, the quality approval test routine for ultrasound therapy transmission assessment combines manual evaluation, threshold-based assessments, machine learning analysis, statistical analysis, signal processing techniques, quantitative metrics and reference standards to determine the overall quality of the ultrasound therapy transmission. The routine enables skilled practitioners within the art to effectively evaluate and ensure the suitability of the ultrasound transmission for generating the intended ultrasound therapy.

In an embodiment, the ultrasound treatment process further comprises, before generating the ultrasound therapy, setting ultrasound therapy parameters at least partly based on real-time or near real-time data from one or more physiological sensors; the therapy parameters including one or more of pulsing frequency, pulse train frequency, number of treated spots, duration, treatment zone area.

In an embodiment, the ultrasound treatment process further comprises, after generating the ultrasound therapy, adjusting the ultrasound therapy parameters during the treatment process if needed at least partly based on updated real-time or near real-time data from one or more physiological sensors.

In an embodiment, the instructions further comprise receiving a health assessment of the user based on received health status data.

In an embodiment, the ultrasound therapy parameters comprise one or more of pulsing frequency, pulse train frequency, number of treated spots, duration, and treatment zone are.

In an embodiment, the device further comprises a communication system.

In an embodiment, the instructions further comprise receiving a health assessment of the user based on received health status data.

In an embodiment, the device is further connected to a device comprising a display screen further comprising a user interface to receive input information about the target therapy area, wherein the input information comprises size and shape of the target therapy area.

In an embodiment, the device further comprises a user-friendly interface allowing the user to interact with the device, monitor therapy progress, adjust settings, and receive alerts or updates.

In an embodiment, the communication system is configured to relay therapy data and receive remote operational commands.

In an embodiment, the device further comprises one or more physiological sensors, wherein the one or more physiological sensors are selected from a group consisting of one or more minimally invasive sensors including but not limited to an electrocardiography (ECG) sensor, photoplethysmography (PPG) sensor, seismocardiography (SCG) sensor, ballistocardiography (BCG) sensor, phonocardiography (PCG) sensor, ultrasonic sensor, temperature sensor, blood pressure sensor, bioimpedance sensor, electromyography (EMG), blood biomarker, pacemaker, glucose sensor, cochlear implants, implantable defibrillators, electroencephalogram (EEG), pulse oximeters, deep brain stimulators, retina implants, intracardiac pressure monitoring sensors and/or a piezoelectric sensor.

In an embodiment, the device further comprises a circuitry to automatically shut off the device upon detection of one or more of excessive heat, pain, and an indicator of potential harm.

In an embodiment, the device further comprises an emergency stop button to allow the user to terminate the ultrasound therapy as and when required.

In an embodiment, the device further comprises wherein the transmission quality test is used to determine the quality of the ultrasound transmission or the proximity of the transducers to the skin.

In an embodiment, the target therapy area comprises at least one of a thoracic region, head region, kidney region, a lung region, a lower limb region, a neck region, a shoulder region, an upper limb region, an abdominal region, and a back region of the user.

In an embodiment, the array of ultrasound transducer units is configured to operate directly through the skin of the user near the target therapy area or indirectly through one or more of an impedance matching material, gel, and fluid near the target therapy area.

In an embodiment, the instructions further include determining whether to not utilize, partially utilize, or fully utilize each of the array of transducer units and activating a selection of the array of transducer units for beam steering of the ultrasound and shifting focal points of the ultrasound to a desired location of the target therapy area.

In an embodiment, the instructions include adjusting a phase and a magnitude of each of the ultrasound transducers in the array of ultrasound transducers.

In an embodiment, the device further comprises a minimally invasive sensor, and the instructions include acquiring through the minimally invasive sensor one or more of: cardiac function information, neuronal function information, pulmonary function information, renal function information, peripheral arterial function, and deep vein thrombosis (DVT) function information of the user.

In an embodiment, the device further comprises a vibration sensor on a side of the wearable housing facing away from the skin-facing side, wherein the instructions include detecting and removing of vibration disturbances sensed by the vibration sensor with receiving and sending signals.

In an embodiment, the instructions include promoting one or more of angiogenesis, reducing inflammation, improving fibrosis, tubular injury, neovascularizing, and ameliorating inflammatory processes in the target therapy area.

In an embodiment, the device further comprises an inertial measurement unit (IMU) sensor, and the instructions include determining with the IMU sensor at least one of whether the ultrasound therapy device is correctly positioned on the skin and whether the user is moving.

In an embodiment, the housing is designed to contour to one or more of the user's chest, head, thorax and lower-limb region.

In an embodiment, the instructions include comparing one or more of pulmonary health, renal health, peripheral artery health, neuronal health, cardiac health and/or deep vein thrombosis health of the user in one or more of the target therapy areas over time to determine the efficacy of the ultrasound therapy.

In an embodiment, the processor is configured to update the ultrasound therapy based on the determined efficacy of the ultrasound therapy over time.

In an embodiment, the wearable housing is rigid or semi-rigid.

In an embodiment, the wearable housing comprises an adjustable strapping mechanism for secure attachment to the user's body.

In an embodiment, the wearable housing comprises an adhesive patch.

In an embodiment, the processor executes instructions to initiate shockwave therapy with an energy level of less than 0.38 mJ/mm².

In an embodiment, the grouping of ultrasound transducers is arranged in an array, wherein the array configuration comprises a specific arrangement of at least one of the following patterns: a linear array for focused ultrasound therapy; a curved array arranged in a concave shape to follow the contour of the body in irregular surfaces; and an annular array following an annular pattern.

In an embodiment, the device is further configured to provide an extracorporeal shockwave treatment in parallel with chemotherapy for cancer treatment to minimize the risk of cancer therapy-induced cardiomyopathies and/or congestive heart failure.

In an embodiment, the device is further configured to provide an extracorporeal shockwave treatment in parallel with chemotherapy for cancer treatment wherein the treatment is administered prior to each chemotherapy session during the cancer treatment regimen.

In an embodiment, the extracorporeal shockwave treatment is synchronized with chemotherapy sessions using a smart algorithm that dynamically adjusts the timing and intensity of shockwaves based on the specific amount and type of chemotherapy drugs administered.

In an embodiment, the device further comprises a control unit and a user interface allowing healthcare professionals to program and customize the initial shockwave treatment parameters based on individual patient profiles.

In an embodiment, extracorporeal shockwave treatment is administered using adjustable shockwave parameters including one or more of frequency, intensity, and duration, tailored to individual patient needs and clinical presentations.

An aspect relates to a method for generating ultrasound treatment to a target therapy area of a user, the method comprising the steps of: providing the user with an ultrasound therapy device containing a housing with ultrasound transducers on a skin-facing side of the housing, an impedance-matching material, gel or fluid, and a user interface; applying onto the user the impedance matching material, gel, or fluid at or near a target therapy area; holding by the user or fastening within a wearable structure the ultrasound therapy device against the impedance matching material, gel, or fluid of the user at or near the target therapy area; sensing, by the ultrasound therapy device, and receiving, by a user interface, information regarding at least some of a location of the target therapy area, a location of the ultrasound therapy device relative to the target therapy area, an ultrasound transmission quality of the impedance matching material, gel or fluid and skin contact, a type of ultrasound therapy to perform, and a health status of the user; activating the ultrasound therapy by the user; and performing the ultrasound therapy on the user based on the information.

In an embodiment, the method further comprises the steps of: mapping out a sub-skin layout of the target therapy area using the ultrasound transducers or other sensors on the device; labeling the features identified in the sub-skin layout; and performing the ultrasound therapy on a portion of the sub-skin layout that matches a pre-determined label.

In an embodiment, the method further comprises the step of: focusing a laser from the ultrasound therapy device or another device below the user's skin near the target therapy area to create an ultrasound emanating from a focal point of the laser by means of laser energy absorbance and the resulting rapid thermal expansion which generates ultrasound waves detected by the ultrasound transducers in the device, utilized for therapy guidance and sub-skin layout mapping allowing for precise targeting and monitoring of therapy area.

In an embodiment, the method further comprises the step of terminating the ultrasound therapy upon depression, actuation, or toggling of an emergency stopper on or near the ultrasound therapy device.

In an embodiment, the target therapy area comprises at least one of a thoracic region, head region, kidney region, a lung region, a lower limb region, a neck region, a shoulder region, an upper limb region, an abdominal region, and a back region of the user.

In an embodiment, the ultrasound therapy is performed to initiate shockwave therapy with an energy level of less than 0.38 mJ/mm² to induce angiogenesis in a cardiac tissue of the user.

In an embodiment, the ultrasound therapy is administered in a population with above average risk factors for developing a stroke.

In an embodiment, the ultrasound therapy is performed using a shockwave therapy parameter with an energy level of less than 0.04 mJ/cm² prior to administering a chemotherapy drug to the user.

In an embodiment, the ultrasound therapy is performed during a drug-coated balloon interventional procedure.

In an embodiment, the method further comprises the steps of receiving information about the ultrasound therapy and controlling the ultrasound therapy through the user interface.

In an embodiment, the method further comprises the step of implementing gating procedures.

An aspect relates to a system for shockwave therapy utilizing an ultrasound system, comprising: an ultrasound therapy module configured to generate ultrasound therapy information, wherein the ultrasound therapy information includes location data, frequency data, spatial average, temporal average data, duty cycle data, and therapy duration data; a shockwave generation module configured to generate shockwaves as a type of shockwave therapy, wherein a shockwave is defined as a single pulse with a wide frequency range of approximately 150 kHz up to 100 MHz, a high pressure amplitude of up to 150 MPa, a low tensile wave of up to −25 MPa, a small positive pulse width lower than 1000 nanoseconds (1 μs) and a total pulse width lower than 10 μs, and a short rise time of up to a few hundred nanoseconds; a treatment parameter adjustment module configured to adjust shockwave parameters as part of the ultrasound therapy information, including the number of spots to treat, the number of shocks per spot, positive peak duration and amplitude, negative tensile peak duration and amplitude, pulse duration, frequency of pulse repetition, pulse train frequency, total therapeutic session duration, and treatment frequency occurrence and duration; a pulse train control module configured to control the application of multiple shocks into a damaged area by dividing the target area into spots and applying multiple shocks to each spot, wherein a group of shocks applied on the same spot is referred to as the train of pulses, and the time between shocks applied on the same spot determines the final pulse repetition frequency of the treatment, while the time between consecutive train of pulses for the treatment of two consecutive target spots determines the pulse train frequency of the therapy; a therapeutic outcome monitoring module configured to monitor and analyze the therapeutic outcomes of shockwave therapy based on the adjusted shockwave parameters; and a user interface module configured to provide a graphical user interface for adjusting shockwave parameters and monitoring therapeutic outcomes.

An aspect relates to a non-invasive ultrasound therapy system for training a machine learning model to optimize ultrasound therapy parameters, the system comprising: a computing device or cloud system connected to receive post-ultrasound therapy health outcome data comprising changes in one or more of a cardiac function, quality of life and angiogenesis, patient information data comprising demographic and medical history data of patients and ultrasound therapy parameter data comprising one or more of pulsing frequency, pulse train frequency, number of treated spots, duration and treatment zone area from a patient population comprising of suspected or confirmed cardiovascular disease patients from multiple clinical study sites, wherein the computing device or cloud system comprises a processor to execute a plurality of instructions comprising: separately match the post-ultrasound therapy health outcome patient data with the patient information data and ultrasound therapy parameter data; generate features from at least one portion of the patient information or ultrasound therapy parameter data using manual methods or unsupervised machine learning methods; and train a machine learning model based on the generated features to optimize ultrasound therapy parameters using unsupervised or supervised machine learning methods, wherein the ultrasound therapy parameters are optimized to increase the likelihood of achieving a shockwave therapeutic effect comprising angiogenesis and neovascularization.

In an embodiment, the instructions further comprise storing the machine learning model in a format compatible to be implemented on a portable computing device.

An aspect relates to a device for generating and delivering ultrasound treatment to a target therapy area of a user. The device includes a housing, an array of ultrasound transducer units, a user interface, a memory, and a processor. The array of ultrasound transducer units is placed on the skin-facing side of the housing. The array of ultrasound transducer units generates and delivers ultrasound therapy to a target therapy area of a user. The user interface receives input information from the user about the target therapy area. The input information comprises size and shape of the target therapy area. The memory stores a plurality of ultrasound therapy instructions. The processor processes the ultrasound therapy instructions. The ultrasound therapy instructions comprising: determining the location and the depth of the target therapy area based on the input information; calculating an optimal configuration for the array of ultrasound transducer units based on the location, depth, size, and shape of the target therapy area; transmitting ultrasound therapy information to the ultrasound transducer units; generating an ultrasound therapy by the array of ultrasound transducer units in the target therapy area of the user based on the optimal configuration of the ultrasound transducer units; sending and receiving ultrasonic waves from the ultrasound transducer units to test an expected throughput of the impedance matching material, gel or fluid, and skin contact; determining if expected throughput is allowable based on a pre-existing threshold; and instructing the processor to start ultrasound therapy if the expected throughput is allowable.

In an embodiment, the device includes a feedback system that is configured to receive feedback from one or more of the user, and a plurality of physiological sensors to adjust the ultrasound therapy as needed during treatment.

In an embodiment, the device includes a circuitry that is configured to automatically shut off the device upon detection of one or more of excessive heat, pain, and an indicator of potential harm. Potential harm indicators may include but are not limited to excessive pressure, tension or strain exerted on the body measured by incorporated sensors, biometric feedback given by physiological sensors incorporated in the device capable of monitoring vital signs and able to show abnormalities that could show potential distress, or user feedback by incorporating buttons or controls that allow the user to provide feedback about their comfort level or any perceived harm.

In an embodiment, the device includes an emergency stop button that is configured to allow the user to terminate the ultrasound therapy as and when required.

In an embodiment, the device includes one or more of a rechargeable battery, and a wireless charging system that are configured to charge one or more components of the device and reduce the need for constant power source replacement.

In an embodiment, the target therapy area includes but are not limited to a kidney region, a lung region, a lower limb region, a neck region, a shoulder region, an upper limb region, an abdominal region, and a back region of the user.

In an embodiment, the array of ultrasound transducer units configured to operate directly through the skin of the user near the target therapy area or indirectly through one or more of the impedance matching material, gel, and fluid near the target therapy area.

One aspect of the present disclosure relates to an ultrasound therapy device for generating ultrasound therapy. The ultrasound therapy device includes a wearable structure, a grouping of ultrasound transducer units, a tightening mechanism, a memory, and a processor. The wearable structure is securable to a user to transmit the ultrasound to a target therapy area of a user including at least one of a kidney region, a lung region, and a lower limb of the user. The ultrasound transducer units are attachable and repositionable in the wearable structure to generate and deliver the ultrasound to the target region. The ultrasound transducer units are arranged in an array. The array of ultrasound transducer units is mechanically moved within the wearable structure and is in contact with a material to facilitate penetration of ultrasound into the user's body. Adequate adhesion to the user's skin to facilitate ultrasound tissue penetration is assessed prior to ultrasound transmission. The tightening mechanism adjusts tightness. The ultrasound therapy device is connected to at least one minimally invasive physiological sensor to collect health status data of the user. A memory includes ultrasound therapy instructions. A processor processes the ultrasound therapy instructions. The ultrasound therapy instructions comprising: determining a non-acute health status of the user based on health status data of the user received by the ultrasound therapy device or a computing device from a minimally invasive physiological sensor; receiving ultrasonic data from the ultrasound transducer units; determining a location of the array of ultrasound transducer units on the user's body; transmitting ultrasound therapy information to the one or more ultrasound transducer units; and generating ultrasound therapy in a target therapy area of the user, after a non-acute health state of the user and correct location of the array has been established.

In an embodiment, the minimally invasive physiological sensor is selected from a group consisting of one or more minimally invasive sensors including but not limited to an electrocardiography (ECG) sensor, photoplethysmography (PPG) sensor, seismocardiography (SCG) sensor, ballistocardiography (BCG) sensor, phonocardiography (PCG) sensor, ultrasonic sensor, temperature sensor, blood pressure sensor, bioimpedance sensor, electromyography (EMG), blood biomarker, pacemaker, glucose sensor, cochlear implants, implantable defibrillators, electroencephalogram (EEG), pulse oximeters, deep brain stimulators, retina implants, intracardiac pressure monitoring sensors and/or a piezoelectric sensor. Minimally invasive sensors as used herein include non-invasive sensors.

In an embodiment, the ultrasound therapy information includes at least one of frequency, temporal average, duty cycle, and therapy duration.

In an embodiment, the ultrasound therapy device includes a minimally invasive sensor for minimally invasively acquiring one or more of: pulmonary function information, renal function information, peripheral arterial function, and deep vein thrombosis (DVT) function information of the user.

In an embodiment, a vibration sensor is facing away from the user's skin to enable the detection of environmental acoustic disturbances that could interfere with receiving and sending signals.

In an embodiment, the pulmonary function information comprises one or more minimally invasive data from the lungs and self-reported pulmonary health data.

In an embodiment, the renal function information comprises one or more minimally invasive data from the kidney and self-reported kidney health data.

In an embodiment, the deep vein thrombosis (DVT) function information comprises one or more minimally invasive data from the lower limb and self-reported lower limb health data.

In an embodiment, the peripheral arterial function information comprises one or more minimally invasive data from the lower limb and self-reported lower limb health data.

In an embodiment, the processor is configured to receive pulmonary function information, and transmit ultrasound therapy information to the one or more ultrasound transducer units for generating ultrasound therapy.

In an embodiment, the processor is configured to receive renal function information, and transmit ultrasound therapy information to the one or more ultrasound transducer units for generating ultrasound therapy.

In an embodiment, the processor is configured to receive deep vein thrombosis (DVT) function information and transmit ultrasound therapy information to the one or more ultrasound transducer units for generating ultrasound therapy.

In an embodiment, the processor is configured to receive peripheral arterial function information and transmit ultrasound therapy information to the one or more ultrasound transducer units for generating ultrasound therapy.

In an embodiment, the ultrasound therapy information is configured to induce pulmonary rehabilitative effects.

In an embodiment, the ultrasound therapy information is configured to promote one or more of angiogenesis, reduce inflammation, reduce fibrosis and tubular injury, increase neovascularization, and ameliorate inflammatory processes.

In an embodiment, the ultrasound therapy information is configured to diminish sodium reabsorption at different sites in the nephron, thereby increasing urinary sodium and water losses, and inhibit water reabsorption by blocking vasopressin receptors along the connecting tubule and collecting duct with the goal of inducing negative body fluid balance.

In an embodiment, the ultrasound therapy is administered in one or more patients diagnosed with one or more of bronchitis, Chronic Obstructive Pulmonary Disease, Cystic Fibrosis, Emphysema, Idiopathic Pulmonary Fibrosis, flu, lung cancer, obstructive sleep apnea, pleurisy, Tuberculosis, pulmonary congestion, congestive organ failure, kidney diseases, and peripheral artery disease.

In an embodiment, the ultrasound therapy device includes a circuit board to connect the one or more ultrasound transducer units, the minimally invasive sensor(s), the positioning mechanism, and the processor.

In an embodiment, the ultrasound therapy device comprises a proximity sensor.

In an embodiment, the proximity sensor is a pressure sensor for measuring the pressure that the one or more ultrasound transducer units apply on the user's skin.

In an embodiment, the ultrasound therapy device includes an inertial measurement unit (IU) sensor configured to determine whether the ultrasound therapy device is correctly positioned on the user's skin and/or determines user movement.

In an embodiment, the ultrasound therapy device includes a housing for accommodating at least a part of one or more ultrasound transducer units, the proximity sensor, and the processor.

In an embodiment, the processor is configured to analyze one or more of the pulmonary function information, the renal function information, and/or the deep vein thrombosis (DVT) function of one or more of the lung, the kidney, and the lower limb of the user and determine one or more locations on the lung, the kidney and the lower limb of the user where the one or more of the ultrasound signals are focused based on ultrasonic sensor data of the one or more ultrasound transducer units.

In an embodiment, the circuit board is a PCB connected to one or more of: an analog-to-digital converter (ADC) for converting analog ultrasonic data into digital data; a micro-controlling unit with a power and data transmission port; one or more large bandwidth operational amplifiers circuits; a plurality of digital buffers; at least two signal mixers for precise doppler calculation; a plurality of filters suitable for an operating range of a piezoelectric ultrasonic sensor; a plurality of bidirectional drivers for a micro linear actuator and a servo motor; and a plurality of headers and a plurality of PWM lines to provide the power to the micro linear actuator and the servo motor.

In an embodiment, the ultrasound therapy device includes a boost circuit for providing power feed to the one or more ultrasound transducer units.

In an embodiment, the boost circuit comprises a low equivalent series resistance (ESR) capacitor and utilizes an accumulated charge on high capacitance.

In an embodiment, the ultrasound therapy information comprises location data, frequency data, spatial average temporal average data, duty cycle data, and therapy duration data.

In an embodiment, the array is configured to direct the ultrasound beam to the one or more regions of the lung the kidney, and the lower limb and execute the ultrasound therapy information in the one or more regions.

In an embodiment, the ultrasound therapy device is configured to compare the pulmonary, renal, deep vein thrombosis or peripheral arterial health of the user in one or more regions over time to determine the efficacy of the ultrasound therapy.

In an embodiment, the ultrasound therapy device is configured to update the ultrasound therapy based on the observed efficacy and safety of the ultrasound therapy over time.

In an embodiment, the ultrasound therapy device is configured to create a map of the user's lungs, kidneys, and lower limbs.

In an embodiment, the ultrasound therapy device is configured to collect information on the user's health condition through a questionnaire and/or patient health database.

In an embodiment, the ultrasound therapy device includes a pressure adjustment mechanism configured to adjust the tightness of the ultrasound transducer units to the user's skin.

In an embodiment, the ultrasound therapy device receives power through a cable from an external electric power system.

In an embodiment, the ultrasound therapy device includes an impedance matching material, gel, or fluid in the form of removable ultrasound gel that is connected to the skin-facing side of the ultrasound therapy device.

Accordingly, one advantage of this example embodiment is that it provides a portable, cost-effective, and efficient wearable and automated ultrasound therapy device to obtain and measure pulmonary function, renal function, peripheral arterial function, and deep vein thrombosis (DVT) function by generating ultrasound therapy.

Accordingly, one advantage of this example embodiment is that it does not require a medical professional and/or assistant to have any specific abilities or training for treating kidney disease, inducing pulmonary rehabilitative effects, peripheral artery disease, and deep vein thrombosis.

Accordingly, one advantage of this example embodiment is that it provides a wearable and automated ultrasound therapy device that can reduce the requirement for hospital-based visits and reduce the number of resources needed by the hospital to treat a patient.

Accordingly, one advantage of this example embodiment is that it automates the continuous positioning of the ultrasound transducer unit and the acquisition of sensor data.

Accordingly, one advantage of this example embodiment is that the continuous positioning of the ultrasound transducer unit is automated with an artificial intelligence-based system approach such as a Case-Based Expert System, and/or a fuzzy logic control system.

Accordingly, one advantage of this example embodiment is that it provides a personalized ultrasound therapy parameter that can be calculated by a machine learning model. Consequently, the ultrasound therapy device can be configured to continuously identify, and scan regions of the kidneys, lungs, and lower limbs of the user that need to be treated and apply region-based ultrasounds.

Other embodiments and advantages will become readily apparent to those skilled in the art upon viewing the drawings and reading the detailed description hereafter, all without departing from the spirit and the scope of the disclosure. The drawings and detailed descriptions presented are to be regarded as illustrative in nature and not in any way as restrictive.

The present disclosure further relates to a device configured to provide a semi-automated ultrasound treatment. Firstly, the user can receive ultrasound therapy information such as disease type that needs to be treated and therapy region, e.g. ischemia and/or organ muscle stiffness. The user can position the device to the therapy region using previously mapped out organ regions from a first-time use calibration process. Then the device can provide the ultrasonic data and/or electronic stethoscope data (and/or other minimally invasive physiological parameter data of the user) from the therapy region to the user. The user can therefore analyze severity of the disease. The risk analysis may be assessed by a risk assessment machine learning model and/or measurement of health function of the user. Then the user can identify the ultrasound therapy parameters such as intensity, duration, and/or pulsation frequency in one or more regions based on the user's characteristics such as body size, age, type, and/or severity of organ disease (e.g. organ muscle stiffness may require an ultrasound therapy to relax the muscle and ischemia may require an ultrasound therapy for regenerative therapy). Lastly, the user can administer the ultrasound therapy based on the identified therapy needs.

In some aspects, the techniques described herein relate to an ultrasound therapy device for generating ultrasound therapy, the ultrasound therapy device including: a housing including a skin-facing side; a grouping of ultrasound transducer units on the skin-facing side to generate and deliver ultrasound to a target therapy area of a user including at least one of a kidney region, a lung region, and a lower limb of the user, the grouping of ultrasound transducer units configured to operate directly through skin of the user near the target therapy area or indirectly through an impedance matching material, gel or fluid near the target therapy area; a communications bus to receive from a minimally invasive physiological sensor a health status data of the user; memory including ultrasound therapy instructions; and a processor for processing the ultrasound therapy instructions, the ultrasound therapy instructions including: determining a non-acute health status of the user based on the health status data of the user received from the minimally invasive physiological sensor; receiving ultrasonic data from the ultrasound transducer units; determining a body location of the grouping of ultrasound transducer units from the ultrasonic data; transmitting ultrasound therapy information to the ultrasound transducer units; and generating ultrasound therapy by the ultrasound transducer units in a target therapy area of the user, after a non-acute health state of the user and the body location of the grouping has been established and determined to be acceptable.

In some aspects, the techniques described herein relate to an ultrasound therapy device, wherein at least some of the ultrasound transducer units of the grouping can be partially or fully utilized to allow for beam steering of the ultrasound and shifting focal points of the ultrasound to a desired location of the target therapy area.

In some aspects, the techniques described herein relate to an ultrasound therapy device, further including: a pressure sensor to measure proximity of the ultrasound transducer units to the skin of the user to determine adequate skin-transducer contact, wherein the determining the body location of the grouping of ultrasound transducer units is further based on readings from the pressure sensor.

In some aspects, the techniques described herein relate to an ultrasound therapy device, wherein the processor generates an ultrasound using phased arrays of the ultrasound transducer units by adjusting a phase and a magnitude of each ultrasound transducer.

In some aspects, the techniques described herein relate to an ultrasound therapy device, wherein the grouping of ultrasound transducers is arranged in an array.

In some aspects, the techniques described herein relate to an ultrasound therapy device, wherein the impedance matching material, gel or fluid is ultrasound gel or an ultrasound gel patch.

In some aspects, the techniques described herein relate to an ultrasound therapy device, wherein the minimally invasive physiological sensor is selected from the group consisting of minimally invasive sensors.

In some aspects, the techniques described herein relate to an ultrasound therapy device, wherein the ultrasound therapy information includes at least one of frequency, temporal average, duty cycle, and therapy duration.

In some aspects, the techniques described herein relate to an ultrasound therapy device, further including: a wearable structure securable to the user, wherein the housing is attachable and repositionable in the wearable structure to target the target therapy area.

In some aspects, the techniques described herein relate to an ultrasound therapy device, further including: a tightening mechanism to adjust tightness of the wearable structure around the body of the user and secure the ultrasound therapy device in position to target the target therapy area and prevent unwanted dispersion of the ultrasound.

In some aspects, the techniques described herein relate to an ultrasound therapy device, further including: sending and receiving ultrasonic waves from the ultrasound transducers to test expected throughput of the impedance matching material, gel or fluid and skin contact; determine if expected throughput is allowable based on a pre-existing threshold; and instruct the processor to start ultrasound therapy if expected throughput is allowable.

In some aspects, the techniques described herein relate to an ultrasound therapy device, wherein the housing is part of a handheld computing device or attachment of the handheld computing device, and the user can interact with a touchscreen display in communication with the ultrasound therapy device on the handheld computing device.

In some aspects, the techniques described herein relate to an ultrasound therapy device, wherein the handheld computing device is a smartphone.

In some aspects, the techniques described herein relate to an ultrasound therapy device, including a minimally invasive sensor for minimally invasively acquiring one or more of: pulmonary function information, renal function information, peripheral arterial function, deep vein thrombosis (DVT) function information of the user.

In some aspects, the techniques described herein relate to an ultrasound therapy device, wherein a second vibration sensor is facing away from the skin-facing side and the processor is configured to enable detection and removal of surrounding acoustic disturbances sensed by the second vibration sensor with receiving and sending signals.

In some aspects, the techniques described herein relate to an ultrasound therapy device, wherein the pulmonary function information includes one or more minimally invasive, imaging and/or biomarker data from the lungs and self-reported pulmonary health data.

In some aspects, the techniques described herein relate to an ultrasound therapy device, wherein the renal function information includes one or more minimally invasive, imaging and/or biomarker data from the kidney and self-reported kidney health data.

In some aspects, the techniques described herein relate to an ultrasound therapy device, wherein the deep vein thrombosis (DVT) function information includes one or more minimally invasive, imaging and/or biomarker data from the lower limb and self-reported lower limb health data.

In some aspects, the techniques described herein relate to an ultrasound therapy device, wherein the processor is configured to receive pulmonary function information and transmit ultrasound therapy information to the one or more ultrasound transducer units for generating ultrasound therapy.

In some aspects, the techniques described herein relate to an ultrasound therapy device, wherein the processor is configured to receive renal function information and transmit ultrasound therapy information to the one or more ultrasound transducer units for generating ultrasound therapy.

In some aspects, the techniques described herein relate to an ultrasound therapy device, wherein the processor is configured to receive deep vein thrombosis (DVT) function information, and transmit ultrasound therapy information to the one or more ultrasound transducer units for generating ultrasound therapy.

In some aspects, the techniques described herein relate to an ultrasound therapy device, wherein the ultrasound therapy information is configured to induce pulmonary rehabilitative effects.

In some aspects, the techniques described herein relate to an ultrasound therapy device, wherein the processor carries out instructions to promote one or more of angiogenesis, reduce inflammation, improve renal fibrosis and tubular injury, neovascularization, and ameliorating inflammatory processes.

In some aspects, the techniques described herein relate to an ultrasound therapy device, wherein the ultrasound therapy is configured to treat one or more of bronchitis, Chronic Obstructive Pulmonary Disease, Cystic Fibrosis, Emphysema, Idiopathic Pulmonary Fibrosis, flu, lung cancer, obstructive sleep apnea, pleurisy, Tuberculosis, pulmonary congestion, kidney diseases, and peripheral artery disease.

In some aspects, the techniques described herein relate to an ultrasound therapy device, further including: a circuit board in communication at least one of the ultrasound transducer units, a pressure sensor, a vibration sensor, a positioning mechanism, and the processor.

In some aspects, the techniques described herein relate to an ultrasound therapy device, wherein a pressure sensor is configured to measure pressure that the ultrasound transducer units apply on the skin.

In some aspects, the techniques described herein relate to an ultrasound therapy device, further including an inertial measurement unit (IN/U) sensor configured to determine at least one of whether the ultrasound therapy device is correctly positioned on the skin and whether the user is moving. The housing accommodates at least a part of each of the ultrasound transducer units, a pressure sensor, and the processor.

In some aspects, the techniques described herein relate to an ultrasound therapy device, wherein the processor is configured to analyze one or more of the pulmonary function information, the renal function information, and/or the deep vein thrombosis (DVT) function of one or more of the lung, the kidney, and the lower limb of the user and determine one or more locations on the lung, the kidney, and the lower limb of the user where the one or more of the ultrasound are focused based on ultrasonic sensor data of one or more ultrasound transducer units.

In some aspects, the techniques described herein relate to an ultrasound therapy device, wherein the circuit board is a PCB connected to one or more of: an analog-to-digital converter (ADC) for converting analog ultrasonic data into digital data; a micro-controlling unit with a power and data transmission port; large bandwidth operational amplifiers circuit; a plurality of digital buffers; at least two signal mixers for precise doppler calculation; a plurality of filters suitable for an operating range of a piezoelectric ultrasonic sensor; a pulse width modulation control; a plurality of bidirectional drivers for a micro linear actuator and a servo motor; and a plurality of headers and a plurality of PWM lines to provide the power to the micro linear actuator and the servo motor.

In some aspects, the techniques described herein relate to an ultrasound therapy device, including a boost circuit for providing power feed to the one or more of the ultrasound transducer units.

In some aspects, the techniques described herein relate to an ultrasound therapy device, wherein the boost circuit includes a low equivalent series resistance (ESR) capacitor and utilizes an accumulated charge on high capacitance.

In some aspects, the techniques described herein relate to an ultrasound therapy device, wherein the ultrasound therapy information includes location data, frequency data, spatial average temporal average data, duty cycle data, and therapy duration data.

In some aspects, the techniques described herein relate to an ultrasound therapy device, wherein the ultrasound therapy device is configured to compare pulmonary or renal health of the user in one or more regions over time to determine efficacy of the ultrasound therapy.

In some aspects, the techniques described herein relate to an ultrasound therapy device, wherein the processor is configured to update the ultrasound therapy based on the determined efficacy of the ultrasound therapy over time.

In some aspects, the techniques described herein relate to an ultrasound therapy device, wherein the processor is configured to create a map of the user's lung, kidney, and the lower limb from signal received by the ultrasound transducers or other sensors.

In some aspects, the techniques described herein relate to an ultrasound therapy device, wherein the processor is configured to collect information on a health condition of the user through at least one of a questionnaire and a patient health database.

In some aspects, the techniques described herein relate to an ultrasound therapy device, wherein the wearable structure includes a pressure adjustment mechanism configured to adjust proximity of the transducer units to the user's skin.

In some aspects, the techniques described herein relate to an ultrasound therapy device, wherein the ultrasound therapy device receives power through a cable from an external electric power system.

In some aspects, the techniques described herein relate to an ultrasound therapy device, wherein the ultrasound therapy device includes a power management unit containing
specific power management circuits, such as but not limited to voltage regulators and convertors, power switches, power sequencing and reset circuits, and sleep mode controllers, and
thermal management circuits configured to:
   optimize power usage and efficient power distribution;
   implement sleep or low-power modes to extend battery life;
   prevent overheating and maintain optimal operation conditions;
   regulate voltage and current levels to safeguard the device's components from potential damage due to excess of current or voltage;

In some aspects, the techniques described herein relate to an ultrasound therapy device, wherein the ultrasound therapy device further includes a removable ultrasound gel patch that is connected to the skin-facing side of the ultrasound therapy device.

In some aspects, the techniques described herein relate to a kit including the ultrasound therapy device, a wearable structure, and a removable ultrasound gel patch.

In some aspects, the techniques described herein relate to a kit including the ultrasound therapy device and ultrasound gel.

In some aspects, the techniques described herein relate to a kit including the ultrasound therapy device, a wearable structure, an impedance matching material, gel or fluid, and fixation pads.

In some aspects, the techniques described herein relate to a method of applying ultrasound therapy by a user, the method including the steps of: providing the user with an ultrasound therapy device containing a housing with ultrasound transducers on a skin facing side of the housing, an impedance matching material, gel or fluid, and a user interface; applying by the user the impedance matching material, gel or fluid at or near a target therapy area including at least one of a kidney region, a lung region, and a lower limb of the user; holding by the user or fastening within a wearable structure the ultrasound therapy device against the impedance matching material, gel or fluid of the user at or near the target therapy area; sensing, by the ultrasound therapy device, and receiving, by the user interface, information regarding at least some of a location of the target therapy area, a location of the ultrasound therapy device relative to the target therapy area, a proximity of the ultrasound transducers to the skin of the user, a type of ultrasound therapy to perform, and a health status of the user; activating the ultrasound therapy by the user; and performing the ultrasound therapy on the user based on the information.

In some aspects, the techniques described herein relate to a method, further including the steps of: mapping out a sub skin layout of the target therapy area using the ultrasound transducers or other sensors on the device; and performing the ultrasound therapy on a portion of the layout.

In some aspects, the techniques described herein relate to a method, further including a step of: focusing a laser from the ultrasound therapy device or another device below the user's skin near the target therapy area to create an ultrasound emanating from a focal point of the laser. Said ultrasound emanating from a focal point of the laser can be detected using ultrasound transducers and analyzed to determine a condition of said focal point.

In some aspects, the system comprises a photoacoustic imaging module and an ultrasound therapy module. The photoacoustic imaging module utilizes laser-induced acoustic waves to generate high-resolution images of biological tissues. In operation, a laser beam is directed onto the tissue, which absorbs the laser energy and undergoes thermal expansion. This rapid expansion generates ultrasound waves that can be detected using an ultrasound transducer. The detected ultrasound signals are processed to reconstruct images of the tissue, providing detailed anatomical and functional information.

In some aspects, the techniques described herein relate to a method, wherein the user interface on a smartphone or smart tablet receives instructions from the user.

In some aspects, the techniques described herein relate to a method, wherein the smartphone or smart tablet is integrated with or physically connected to the ultrasound therapy device.

In some aspects, the method includes a step of receiving, by a feedback system, feedback from one or more of the user, and a plurality of physiological sensors to adjust the ultrasound therapy as needed during treatment.

In some aspects, the method includes a step of allowing, by an emergency stop button, the user to terminate the ultrasound therapy as and when required.

In some aspects, the user-initiated termination of ultrasound therapy through an emergency stopper includes but is not limited to depression by the application of pressure on the designated button, switch or control mechanism on or near the therapy device; actuation by triggering a specific mechanism, sensor or input device integrated into the ultrasound therapy or near the device such as but not limited to a microphone for voice command and/or a camera for gesture recognition or a mobile application control; toggling a switch, lever or other similar element on or near the device.

In some aspects, the target therapy area comprises at least one of a kidney region, a lung region, a lower limb region, a neck region, a shoulder region, an upper limb region, an abdominal region, and a back region of the user.

Another aspect relates to an ultrasound system for generating and delivering ultrasound treatment to a target therapy area of a user. The ultrasound system includes an ultrasound therapy device.

The ultrasound therapy device includes a housing with a skin-facing side, an array of ultrasound transducer units, a user interface, a memory, and a processor. The array of ultrasound transducer units on the skin-facing side. The array of ultrasound transducer units configured to generate and deliver ultrasound therapy to a target therapy area of a user. The user interface receives input information from the user about the target therapy area. The input information comprises size and shape of the target therapy area. The memory stores a plurality of ultrasound therapy instructions. The processor for processing the ultrasound therapy instructions. The ultrasound therapy instructions comprising: determining the location and the depth of the target therapy area based on the input information; calculating an optimal configuration for the array of ultrasound transducer units based on the location, depth, size, and shape of the target therapy area; transmitting ultrasound therapy information to the ultrasound transducer units; generating an ultrasound therapy by the array of ultrasound transducer units in the target therapy area of the user based on the optimal configuration of the ultrasound transducer units; sending and receiving ultrasonic waves from the ultrasound transducer units to test an expected throughput of the impedance matching material, gel or fluid, and skin contact; determining if expected throughput is allowable based on a pre-existing threshold; and instructing the processor to start ultrasound therapy if the expected throughput is allowable.

In an aspect, the ultrasound therapy device includes a feedback system to receive feedback from one or more of the user, and a plurality of physiological sensors to adjust the ultrasound therapy as needed during treatment.

In an aspect, the ultrasound therapy device includes a circuitry to automatically shut off the device upon detection of one or more of excessive heat, pain, and an indicator of potential harm. Potential harm indicators may include but are not limited to excessive pressure, tension or strain exerted on the body measured by incorporated sensors, biometric feedback given by physiological sensors incorporated in the device capable of monitoring vital signs and able to show abnormalities that could show potential distress, or user feedback by incorporating buttons or controls that allow the user to provide feedback about their comfort level or any perceived harm.

In an aspect, the ultrasound therapy device comprises an emergency stop button to allow the user to terminate the ultrasound therapy as and when required.

In an aspect, the ultrasound therapy device includes one or more of a rechargeable battery, and a wireless charging system to charge one or more components of the device and reduce the need for constant power source replacement.

In an aspect, the target therapy area includes but is not limited to a kidney region, a lung region, a lower limb region, a neck region, a shoulder region, an upper limb region, an abdominal region, and a back region of the user.

In an aspect, the array of ultrasound transducer units is configured to operate directly through the skin of the user near the target therapy area or indirectly through one or more of the impedance matching material, gel, and fluid near the target therapy area.

In an aspect, the array of ultrasound transducer units is configured in a grid pattern, distributed across the wearable surface. Each unit within the array is designed to emit ultrasound waves and can be independently activated, allowing for precise control over direction and intensity of the ultrasound beam. Activation of the units can be achieved via mechanical beam steering or electronic beam steering. For mechanical beam steering, the device is equipped with mechanisms that allow for controlled movement and/or rotation of the units in the array such as but not limited to motor, gears or manual adjustable mechanisms. In the case of electronic beam steering, the transducer array remains stationary, and beam direction is controlled through electronic means. Each transducer element within the array is individually activated through electronic control circuitry. By selectively activating specific elements and adjusting their timing and intensity, the ultrasound beam can be electronically focused and steered in desired directions.

In some aspects, the techniques described herein relate to an ultrasound system for generating ultrasound therapy, the ultrasound system including: an ultrasound therapy device, including; a housing including a skin-facing side; a grouping of ultrasound transducer units on the skin-facing side to generate and deliver ultrasound to a target therapy area of a user including at least one of a kidney region, a lung region, and a lower limb of the user, the grouping of ultrasound transducer units configured to operate directly through skin of the user near the target therapy area or indirectly through an impedance matching material, gel or fluid near the target therapy area; and a communications bus to receive from a minimally invasive physiological sensor a health status data of the user, and to exchange information and instructions from a processor and memory; the memory including ultrasound therapy instructions; and the processor for processing the ultrasound therapy instructions, the ultrasound therapy instructions including: determining a non-acute health status of the user based on the health status data of the user received from the minimally invasive physiological sensor; receiving ultrasonic data from the ultrasound transducer units; determining a body location of the grouping of ultrasound transducer units from the ultrasonic data; transmitting ultrasound therapy information to the ultrasound transducer units; and generating ultrasound therapy by the ultrasound transducer units in a target therapy area of the user, after a non-acute health state of the user and the body location of the grouping has been established and determined to be acceptable.

In some aspects, the techniques described herein relate to an ultrasound system for generating ultrasound therapy, the ultrasound system including: an ultrasound therapy device, including; a housing including a skin-facing side; a grouping of ultrasound transducer units on the skin-facing side to generate and deliver at a focal point of ultrasound waves ultrasound to a target therapy area of a user including at least one of a kidney region, a lung region, and a lower limb of the user, the grouping of ultrasound transducer units configured to operate directly through skin of the user near the target therapy area or indirectly through an impedance matching material, gel or fluid near the target therapy area; and a communications bus to receive from a minimally invasive physiological sensor a health status data of the user, and to exchange information and instructions from a processor and memory; the memory including ultrasound therapy instructions; and the processor for processing the ultrasound therapy instructions, the ultrasound therapy instructions including: determining a non-acute health status of the user based on the health status data of the user received from the minimally invasive physiological sensor; receiving ultrasonic data from the ultrasonic transducer units; determining a body location of the grouping of ultrasonic transducer units from the ultrasonic data; transmitting ultrasound therapy information to the ultrasonic transducer units; and generating ultrasound therapy at the focal point of ultrasound waves generated by the ultrasound transducer units in a target therapy area of the user, after a non-acute health state of the user and the body location of the grouping has been established and determined to be acceptable.

In some aspects, the techniques described herein relate to an ultrasound system, wherein at least some of the ultrasound transducer units of the grouping can be partially or fully utilized to allow for beam steering of the ultrasound and shifting focal points of the ultrasound to a desired location of the target therapy area.

In some aspects, the techniques described herein relate to an ultrasound system, further including: a pressure sensor to measure proximity of the ultrasound transducer units to the skin of the user to determine adequate skin-transducer contact, wherein the determining the body location of the grouping of ultrasound transducer units is further based on readings from the pressure sensor.

In some aspects, the techniques described herein relate to an ultrasound system, wherein the processor generates an ultrasound using phased arrays of the ultrasound transducer units by adjusting a phase and a magnitude of each ultrasound transducer.

In some aspects, the techniques described herein relate to an ultrasound system, wherein the grouping of ultrasound transducers is arranged in an array.

In some aspects, the techniques described herein relate to an ultrasound system, wherein the array is arranged two- or three dimensionally.

In some aspects, the techniques described herein relate to an ultrasound system, wherein the impedance matching material, gel or fluid is ultrasound gel or an ultrasound gel patch.

In some aspects, the techniques described herein relate to an ultrasound system, wherein the minimally invasive physiological sensor is selected from the group consisting of minimally invasive sensors.

In some aspects, the techniques described herein relate to an ultrasound system, wherein the ultrasound therapy information includes at least one of frequency, temporal average, duty cycle, and therapy duration.

In some aspects, the techniques described herein relate to an ultrasound system, further including: a wearable structure securable to the user, wherein the housing is attachable and repositionable in the wearable structure to target the target therapy area.

In some aspects, the techniques described herein relate to an ultrasound system, further including: a tightening mechanism to adjust tightness of the wearable structure around the body of the user and secure the ultrasound therapy device in position to target the target therapy area and prevent unwanted dispersion of the ultrasound.

In some aspects, the techniques described herein relate to an ultrasound system, further including sending and receiving ultrasonic waves from the ultrasound transducers to test expected throughput of the impedance matching material, gel or fluid and skin contact; determine if expected throughput is allowable based on a pre-existing threshold; and instruct the processor to start ultrasound therapy if expected throughput is allowable.

In some aspects, the techniques described herein relate to an ultrasound system, wherein the housing is part of a handheld computing device or attachment of the handheld computing device, and the user can interact with a touchscreen display in communication with the ultrasound therapy device on the handheld computing device.

In some aspects, the techniques described herein relate to an ultrasound system, wherein the handheld computing device is a smartphone.

In some aspects, the techniques described herein relate to an ultrasound system, including an minimally invasive sensor for minimally invasively acquiring one or more of: pulmonary function information, renal function information, peripheral arterial function, deep vein thrombosis (DVT) function information of the user.

In some aspects, the techniques described herein relate to an ultrasound system, wherein a second vibration sensor is facing away from the skin-facing side and the processor is configured to enable detection and removal of surrounding acoustic disturbances sensed by the second vibration sensor with receiving and sending signals.

In some aspects, the techniques described herein relate to an ultrasound system, wherein the pulmonary function information includes one or more minimally invasive data from the lungs and self-reported pulmonary health data.

In some aspects, the techniques described herein relate to an ultrasound system, wherein the renal function information includes one or more minimally invasive data from the kidney and self-reported kidney health data.

In some aspects, the techniques described herein relate to an ultrasound system, wherein the deep vein thrombosis (DVT) function information includes one or more minimally invasive data from the lower limb and self-reported lower limb health data.

In some aspects, the techniques described herein relate to an ultrasound system, wherein the processor is configured to receive pulmonary function information and transmit ultrasound therapy information to the one or more ultrasound transducer units for generating ultrasound therapy.

In some aspects, the techniques described herein relate to an ultrasound system, wherein the processor is configured to receive renal function information and transmit ultrasound therapy information to the one or more ultrasound transducer units for generating ultrasound therapy.

In some aspects, the techniques described herein relate to an ultrasound system, wherein the processor is configured to receive deep vein thrombosis (DVT) function information, and transmit ultrasound therapy information to the one or more ultrasound transducer units for generating ultrasound therapy.

In some aspects, the techniques described herein relate to an ultrasound system, wherein the ultrasound therapy information is configured to induce pulmonary rehabilitative effects.

In some aspects, the techniques described herein relate to an ultrasound system, wherein the processor carries out instructions to promote one or more of angiogenesis, reduce inflammation, improve renal fibrosis and tubular injury, neovascularization, and ameliorating inflammatory processes.

In some aspects, the techniques described herein relate to an ultrasound system, wherein the ultrasound therapy is configured to treat one or more of bronchitis, Chronic Obstructive Pulmonary Disease, Cystic Fibrosis, Emphysema, Idiopathic Pulmonary Fibrosis, flu, lung cancer, obstructive sleep apnea, pleurisy, Tuberculosis, pulmonary congestion, kidney diseases, and peripheral artery disease.

In some aspects, the techniques described herein relate to an ultrasound system, further including: a circuit board in communication at least one of the ultrasound transducer units, a pressure sensor, a vibration sensor, a positioning mechanism, and the processor.

In some aspects, the techniques described herein relate to an ultrasound system, wherein the pressure sensor is configured to measure pressure that the ultrasound transducer units applies on the skin.

In some aspects, the techniques described herein relate to an ultrasound system, further including an inertial measurement unit (IMU) sensor configured to determine at least one of whether the ultrasound therapy device is correctly positioned on the skin and whether the user is moving.

In some aspects, the techniques described herein relate to an ultrasound system, wherein the housing accommodates at least a part of each of the ultrasound transducer units, a pressure sensor, and the processor.

In some aspects, the techniques described herein relate to an ultrasound system, wherein the processor is configured to analyze one or more of the pulmonary function information, the renal function information, and/or the deep vein thrombosis (DVT) function of one or more of the lung, the kidney, and the lower limb of the user and determine one or more locations on the lung, the kidney, and the lower limb of the user where the one or more of the ultrasound are focused based on ultrasonic sensor data of one or more ultrasound transducer units.

In some aspects, the techniques described herein relate to an ultrasound system, wherein the circuit board is a PCB connected to one or more of: an analog-to-digital converter (ADC) for converting analog ultrasonic data into digital data; a micro-controlling unit with a power and data transmission port; large bandwidth operational amplifiers circuit; a plurality of digital buffers; at least two signal mixers for precise doppler calculation; a plurality of filters suitable for an operating range of a piezoelectric ultrasonic sensor; a pulse width modulation control; a plurality of bidirectional drivers for a micro linear actuator and a servo motor; and a plurality of headers and a plurality of PWM lines to provide the power to the micro linear actuator and the servo motor.

In some aspects, the techniques described herein relate to an ultrasound system, including a boost circuit for providing power feed to the one or more of the ultrasound transducer units.

In some aspects, the techniques described herein relate to an ultrasound system, wherein the boost circuit includes a low equivalent series resistance (ESR) capacitor and utilizes an accumulated charge on high capacitance.

In some aspects, the techniques described herein relate to an ultrasound system, wherein the ultrasound therapy information includes location data, frequency data, spatial average temporal average data, duty cycle data, and therapy duration data.

In some aspects, the techniques described herein relate to an ultrasound system, wherein the ultrasound therapy device is configured to compare pulmonary or renal health of the user in one or more regions over time to determine efficacy of the ultrasound therapy.

In some aspects, the techniques described herein relate to an ultrasound system, wherein the processor is configured to update the ultrasound therapy based on the determined efficacy of the ultrasound therapy over time.

In some aspects, the techniques described herein relate to an ultrasound system, wherein the processor is configured to create a map of the user's lung, kidney, and the lower limb from signal received by the ultrasound transducers or other sensors.

In some aspects, the techniques described herein relate to an ultrasound system, wherein the processor is configured to collect information on a health condition of the user through at least one of a questionnaire and a patient health database.

In some aspects, the techniques described herein relate to an ultrasound system, wherein the wearable structure includes a pressure adjustment mechanism configured to adjust proximity of the transducer units to the user's skin.

In some aspects, the techniques described herein relate to an ultrasound system, wherein the ultrasound therapy device receives power through a cable from an external electric power system.

In some aspects, the techniques described herein relate to an ultrasound system, wherein the ultrasound therapy device further includes a removable ultrasound gel patch that is connected to the skin-facing side of the ultrasound system.

In some aspects, the techniques described herein relate to a kit including the ultrasound system, a wearable structure, and a removable ultrasound gel patch.

In some aspects, the techniques described herein relate to a kit including the ultrasound system and ultrasound gel.

In some aspects, the techniques described herein relate to a kit including the ultrasound system, a wearable structure, an impedance matching material, gel or fluid, and fixation pads.

In some aspects, the array of ultrasound transducer units is replaced with a single, large, mechanically steerable ultrasound transducer, controlled by the processor to generate and deliver ultrasound therapy with variable frequency, intensity, and duration parameters specifically optimized for a predetermined therapeutic application.

In some aspects, the techniques described herein relate to a non-invasive ultrasound therapy system for training a classifier machine learning model to classify each element of the grid-based mapping of the organ as healthy or treatment area, the system comprising: a computing device or cloud system connected to receive patient information data including demographics and medical history, previously estimated data on the patient's health status and disease progression, and previous classification label in the grid for tissue/organ location and type information, wherein the computing device or cloud system includes a processor configured to execute a plurality of instructions, including: combining patient information data, health status and disease progression information and previously calculated classification in the organ mapping model with the organ/tissue type and location labels into a single dataset; generating features from at least one portion of the collected data that fall within each of at least one temporal window; training a classifier machine learning model based on the generated features to detect and classify each area on the targeted organ map, being able to determine those spots within the organ/tissue mapping grid that need treatment and those spots within the organ/tissue mapping grid that do not need further treatment, and label the energy level of the treatment for each spot on the grid (none, low, medium, high); and storing the trained machine learning model in a format that permits for its deployment in a portable computing device.

In some aspects, the techniques described herein relate to a non-invasive ultrasound therapy system for training a machine learning model to assess patient health status, and disease progression, the system comprising: computing device or cloud system connected to receive patient information data comprising medical history and demographics, past ultrasound therapy sessions information including number of undergone previous sessions, duration, and treated area, received non-invasive sensor signals from elements present in the device, and gold standard measurements of cardiac function (e.g. Ejection Fraction or Myocardial Perfusion Index) wherein the computing device contains a processor configured to execute a plurality of instructions, including: initially merging patient information data, ultrasound therapy past sessions information and received/sensed non-invasive data into a single dataset; separately matching the received/sensed gold standard patient data with the patient information data, past ultrasound therapy information, and recorded non-invasive sensor signals; generating features from at least one portion of the matched data that fall within each of at least one temporal window; training a machine learning model based on the generated features using supervised machine learning algorithms to assess patient health status, and estimate disease progression via the estimation of health biomarkers such as ejection fraction, CCS class in cardiac assessment; and storing the trained machine learning model in a format that allows for its deployment in a wearable and/or portable computing device.

In some aspects, the techniques described herein relate to a non-invasive ultrasound therapy system for training a classifier machine learning model to detect and classify adverse events, the system comprising: a computing device connected to receive patient information data, past ultrasound therapy sessions information, and non-invasive sensor signals; a processor within the computing device configured to execute a plurality of instructions, including: combining patient information data, past ultrasound therapy sessions information, and non-invasive sensor signals into a single dataset; generating features from at least one portion of the collected data that fall within each of at least one temporal window; training a classifier machine learning model based on the generated features to detect and classify adverse events; and storing the trained machine learning model.

In some aspects, the techniques described herein relate to a machine learning model to classify and label tissue location and type in the targeted area, the system comprising: a computing device connected to receive patient information data, non-invasive data recordings and ultrasound therapy parameter data, wherein the computing device comprises a processor to execute a plurality of instructions comprising: generate features from at least one portion of the collected data that fall within each of at least one temporal window; train a machine learning model based on the generated features to map the organ location and create a grid-based map of the area, labeling each grid-position based on the type of tissue recognized by the model; and store the machine learning model.

Other features of the example embodiments will be apparent from the drawings and from the detailed description that follows.

### BRIEF DESCRIPTION OF THE DRAWINGS

The accompanying drawings illustrate some embodiments of systems, methods, and other aspects of the disclosure. Any person of ordinary skill in the art will appreciate that the illustrated element boundaries (e.g., boxes, groups of boxes, or other shapes) in the figures represent an example of the boundaries. In some examples, one element may be designed as multiple elements, or multiple elements may be designed as one element. In some examples, an element shown as an internal component of one element may be implemented as an external component in another and vice versa. Furthermore, the elements may not be drawn to scale. Reference numerals may be selectively repeated across images for clarity or emphasis.

Various embodiments will hereinafter be described in accordance with the appended drawings, which are provided to illustrate, not limit, the scope, wherein similar designations denote similar elements, and in which:

FIG. 1A illustrates a perspective view of an example wearable structure that allows the ultrasound therapy device to target the kidney region, or the lung region (as shown) of the user. FIG. 1B illustrates a perspective view of another example wearable structure that allows the ultrasound therapy device to target the diaphragm or stomach of the user.

FIG. 2 illustrates a perspective view of the wearable structure that allows the ultrasound therapy device to fit around the lower limb of the user, in accordance with one embodiment of the present disclosure.

FIG. 3 illustrates a block diagram of example connections between the various components of the wearable structure configured to fit around one or more of: the kidney region, the lung region, and the lower limb of the user, in accordance with one embodiment of the present disclosure.

FIG. 4 illustrates a bottom view of an example ultrasound therapy device, in accordance with one embodiment of the present disclosure.

FIG. 5 illustrates a side view of the ultrasound therapy device, in accordance with one embodiment of the present disclosure.

FIG. 6 illustrates a monitoring application installed within the ultrasound therapy device in the form of a handheld computing device, in accordance with one embodiment of the present disclosure.

FIG. 7 illustrates a perspective view of the handheld computing device placed against the body or chest of a user, in accordance with at least one embodiment.

FIG. 8 illustrates a first perspective view of the placement of the ultrasound therapy device against the body of the user, in accordance with at least one embodiment.

FIG. 9 illustrates a second perspective view of the placement of the ultrasound therapy device against the chest of the user, in accordance with at least one embodiment.

FIG. 10 illustrates a skin-facing side view of a matrix or array of ultrasound transducer units, in accordance with at least one embodiment.

FIG. 11 depicts an example electronics diagram of an example ultrasound system.

FIG. 12 is a flowchart of an example method of applying automated ultrasound therapy.

FIG. 13 illustrates a perspective view of the wearable ultrasound therapy device that acts as a chest-worn adhesive patch, in accordance with one embodiment of the present disclosure.

FIG. 14 illustrates a flow diagram related to cardiac-specific organ mapping and cardiac-specific health assessment models, in accordance with one embodiment of the present disclosure.

FIG. 15 illustrates a perspective view of the cardiac or organ mapping classification of a grid, in accordance with one embodiment of the present disclosure.

FIG. 16 illustrates a block diagram of a cardiac-specific treatment therapy optimization system, in accordance with one embodiment of the present disclosure.

FIG. 17 illustrates a perspective view of the wearable ultrasound therapy device that acts as a helmet, in accordance with one embodiment of the present disclosure. FIG. 17A depicts an example array arranged in an annular configuration. FIG. 17B depicts a mechanism for adjusting the positioning and adjustment of a curved array.

FIG. 18A illustrates an example of the data matching step in the health assessment machine learning model between gold standard parameters in cardiac health and myocardial function (ejection fraction measured from echocardiography or cardiac MRI), and recorded signals collected from non-invasive sensors in the wearable device presented in this disclosure, in accordance with at least one embodiment. FIG. 18B illustrates an example process of gold standard data as a dependent variable (y) and the generated features from non-invasive recorded data as an independent variable (x) to be used in training a machine learning model to assess health status and disease progression.

FIG. 19 depicts a descriptive flowchart of the steps in the development of the machine learning models involved in the health monitoring phase of the devices presented in this disclosure, in accordance with at least one embodiment.

FIG. 20 illustrates a comprehensive flowchart outlining the sequential steps entailed in the development of the machine learning model employed for optimizing treatment by classifying the treatment area, as per at least one embodiment.

FIG. 21 depicts a descriptive flowchart of the steps in the development of the machine learning model involved in the treatment optimization phase for the generation of treatment parameters presented in this disclosure, in accordance with at least one embodiment.

### DETAILED DESCRIPTION

The present description is best understood with reference to the detailed figures and description set forth herein. Various embodiments of the present system and method have been discussed with reference to the figures. However, those skilled in the art will readily appreciate that the detailed description provided herein with respect to the figures is merely for explanatory purposes, as the present system and method may extend beyond the described embodiments. For instance, the teachings presented and the needs of a particular application may yield multiple alternative and suitable approaches to implement the functionality of any detail of the present systems and methods described herein. Therefore, any approach to implement the present system and method may extend beyond certain implementation choices in the following embodiments.

According to an embodiment herein, the methods herein may be implemented by performing or completing manually, automatically, and/or a combination of thereof. The term “method” refers to manners, means, techniques, and procedures for accomplishing any task including, but not limited to, those manners, means, techniques, and procedures either known to the person skilled in the art or readily developed from existing manners, means, techniques and procedures by persons skilled in the art. The persons skilled in the art will envision many other possible variations within the scope of the present system and method described herein.

One example of ultrasound are shockwaves. Generally, ultrasound is defined as either low intensity or high intensity by determining whether the energy is below or above 1 W/cm2. Further, the low-frequency and high-frequency ultrasound are classified by determining whether the frequency is below or above 1 MHz. The low frequency ultrasound has good penetration that can reach deeper targets and initiate predominantly mechanical effects on cell membranes with negligible temperature increase (<0.01° C.), thereby depolarizing membranes to activate voltage-gated sodium channels and voltage-gated calcium channels and to influence cells' excitability. However, high-frequency ultrasound has a shorter wavelength and better spatial resolution than low-frequency ultrasound. The high-frequency ultrasound is centrally deposited, which is helpful in imaging. Fast attenuation of the high-frequency ultrasound may cause thermal loss and poor penetration when applied to the delivery of skin treatment.

A shockwave can be described as a single pulse with a wide frequency range (from approx. 150 kHz up to 100 MHz), high pressure amplitude (up to 150 MPa), low tensile wave (up to −25 MPa), small pulse width and a short rise time of up to a few hundred nanoseconds. Shockwave Therapy involves the application of multiple shockwaves into the damaged area, by dividing the target area into spots to be treated by the application of multiple shocks.

Shockwave parameters comprise but are not limited to number of spots to treat, number of shocks per spot, positive peak duration and amplitude, negative tensile peak duration and amplitude, pulse duration and frequency of pulse repetition, pulse train frequency, total therapeutical session duration and treatment frequency occurrence and duration.

During shockwave treatment, several shocks are applied per spot. This grouping of shocks per spot is referred to as train of pulses. The time between shocks on the same spot determines the final pulse repetition frequency of the treatment, while the time between consecutive train of pulses for the treatment of two consecutive target spots determines the Pulse Train frequency of the therapy. These adjustable parameters enable precise and targeted treatment for improved therapeutic outcomes in various medical conditions.

The ultrasound therapy module is configured to generate ultrasound therapy information that includes location data, frequency data, spatial average data, temporal average data, duty cycle data, and therapy duration data. The location data specifies the target therapy area where the shockwave therapy is to be administered, such as the myocardium or peripheral tissue. The frequency data represents the wide frequency range of shockwaves, typically spanning from approximately 150 kHz up to 100 MHz. The spatial average data represents the average energy distribution within the target area and can be adjusted within the range of 0.01 to 0.38 mJ/mm² to optimize angiogenesis effects. The temporal average data represents the average energy delivered during the therapy session and can be adjusted within the range of 0.01 to 0.38 mJ/mm² to provide the desired angiogenic response. The duty cycle data indicates the proportion of time that shockwaves are actively generated during each pulse or treatment session and can be adjusted within the range of 10% to 50% to ensure appropriate energy delivery for angiogenesis. The therapy duration data specifies the duration of each therapy session or the total treatment duration, which can be adjusted within the range of 15 minutes to 60 minutes to promote angiogenesis.

To be able to determine the optimal location on the chest of the user where the shock wave transducer unit should be positioned and the shockwave parameters that should be transmitted, pre-defined treatment protocols from Tohoku University of Japan with respect to the shockwave output and the number of shots implemented to each spot and the protocol developed by the University of Essen, Germany, can be used.

If the disease type e.g. turns out to be end-stage coronary artery disease, a treatment of three sessions per week for three weeks with up to 1200 impulses to the patient per session applied to the basal, middle, and apical segments of the left ventricle and not more than 100 impulses applied to one spot with an energy flux of 0.09 mJ/mm2 will reduce the ischemia burden.

If the disease type e.g. turns out to be ischemic heart disease preferably 300 impulses can be delivered to the ischemic area with an energy flux density of 0.38 mJ/mm2 at a frequency of 4 Hz to generate one or more of a regenerative effect through angiogenesis and/or neovascularization.

If the disease type e.g. turns out to be calcified aortic valve leaflets the hardened masses can be destructed by lithotripsy using two shockwaves at 100 kHz and at 3 MHz, respectively, with a time interval of 6 s. The combination of the different frequencies will disrupt the calcium deposits in the aortic valve cusps, avoiding thermal injury.

The shockwave generation module is configured to generate shockwaves as a type of ultrasound therapy. The shockwaves are defined as single pulses with specific characteristics. They have a wide frequency range of approximately 150 kHz up to 100 MHz, a high-pressure amplitude up to 150 MPa, a low tensile wave ranging up to −25 MPa, a small positive pulse width ranging from 10 ns to 1000 ns, and a short rise time ranging of up to a few hundred nanoseconds (ranging from 1 ns to 100 ns). To induce angiogenesis, the shockwave parameters can be adjusted within the given ranges. For example, the positive peak duration can be set within the range of 10 ns to 50 ns, while the positive peak amplitude can be adjusted within the range of 50 MPa to 100 MPa to promote angiogenesis effects. The negative tensile peak duration can be set within the range of 1 μs to 5 μs, and the negative tensile peak amplitude can be adjusted within the range of −5 MPa to −20 MPa to further enhance angiogenesis. The complete pulse duration can be set within the range of 5 μs to 10 μs to ensure controlled energy release. The frequency of pulse repetition can be adjusted within the range of 1 Hz to 5 Hz, and the pulse train frequency can be set to 1 Hz to deliver shockwaves at the optimal timing for angiogenesis. These specific shockwave characteristics, when adjusted within the specified ranges, allow for precise and controlled energy delivery to the target therapy area, promoting angiogenesis.

The treatment parameter adjustment module is configured to adjust shockwave parameters as part of the ultrasound therapy information. The shockwave parameters include the number of spots to treat, the number of shocks per spot, total therapeutic session duration, and treatment frequency occurrence and duration. For inducing angiogenesis, the number of spots to treat can be adjusted within the range of 1 to 60, depending on the size and location of the target area. The number of shocks per spot can be set within the range of 200 to 2000, ensuring adequate energy delivery for angiogenesis. The total therapeutic session duration can be adjusted within the range of 15 minutes to 60 minutes, accommodating various treatment needs. The treatment frequency occurrence can be set within the range of once a month to several times a day, while the treatment frequency duration can be adjusted within the range of 15 minutes to an hour, providing flexibility in treatment scheduling for angiogenesis induction.

In some embodiments, a treatment parameter adjustment module is disclosed as part of the ultrasound therapy system, providing specific examples and instructions on how to adjust shockwave parameters to induce angiogenesis. The treatment parameter adjustment module allows precise customization of shockwave parameters to optimize angiogenesis therapy. The following examples and instructions demonstrate the implementation for angiogenesis treatment:

Number of Spots to Treat: Assess the size and distribution of the target area where angiogenesis is desired. For example, determine that the target area consists of 10 distinct spots. Configure the treatment parameter adjustment module to allow for the selection and adjustment of the number of spots to treat. For example, set the parameter to treat all 10 spots or select a specific subset based on the patient's condition and treatment plan.

Number of Shocks per Spot: Determine the optimal number of shocks required to induce angiogenesis in each treated spot. For example, based on clinical studies and research, determine that 200-1,000 shocks per spot are effective for angiogenesis. Configure the treatment parameter adjustment module to allow for the adjustment of the number of shocks per spot. Ensure that the module provides a range of values to accommodate variations in patient response and treatment requirements.

Shockwave Characteristics: Adjust the shockwave parameters to promote angiogenesis in the treated spots. Consider parameters such as positive peak duration, positive peak amplitude, negative tensile peak duration, negative tensile peak amplitude, and pulse duration. For example, set the positive peak duration to 1 microsecond, positive peak amplitude to 100 MPa, negative tensile peak duration to 3 microseconds, negative tensile peak amplitude to −2 MPa, and pulse duration to 10 microseconds. Configure the treatment parameter adjustment module to allow for precise control and adjustment of these shockwave characteristics.

Frequency of Pulse Repetition: Determine the optimal frequency at which pulses should be repeated to induce angiogenesis. For example, based on previous studies, determine that a pulse repetition frequency (PRF) of around 20 Hz can be effective. Configure the treatment parameter adjustment module to allow for the adjustment of the PRF within a suitable range, such as 5-35 Hz. Ensure that the module provides flexibility to fine-tune the PRF based on patient response and treatment progress.

Pulse Train Frequency: Adjust the pulse train frequency to optimize angiogenesis therapy. Determine the appropriate frequency at which the pulse train should be applied. For example, based on clinical experience, determine that a pulse train frequency (PTF) of 2 Hz is beneficial for angiogenesis. Configure the treatment parameter adjustment module to allow for the adjustment of the PTF within a suitable range, such as 1-5 Hz. Ensure that the module enables healthcare professionals to easily modify the PTF based on individual patient characteristics and treatment requirements.

Total Therapeutic Session Duration: Define the duration of the overall therapeutic session for angiogenesis treatment. Consider factors such as patient comfort, treatment response, and clinical guidelines. For example, set the total therapeutic session duration to 30 minutes. Configure the treatment parameter adjustment module to monitor and control the overall session duration.

Treatment Frequency Occurrence and Duration: Determine the optimal frequency and duration of treatment sessions for effective angiogenesis. Consider patient factors, treatment goals, and medical guidelines. For example, based on clinical evidence, determine that treatment sessions should be conducted twice a week for a duration of six weeks. Configure the treatment parameter adjustment module to allow for the customization and scheduling of treatment sessions.

In some embodiments, a pulse train control module is disclosed as part of the shockwave therapy system, providing specific examples and instructions on how it can be implemented. The pulse train control module enables precise control over the application of shockwaves to a damaged area, facilitating effective treatment interventions.

The following examples and instructions demonstrate implementation:

Spot Division and Identification: Divide the target area into a grid-like pattern, with the resolution of the grid divisions dependent on the approximate size of the treatment area needed. For example, customize the grid resolution based on the specific treatment requirements, such as dividing the target area into a 5×5 grid, resulting in 25 individual spots. Assign unique identifiers to each spot, such as Spot 1, Spot 2, Spot 3, and so on, for tracking and reference purposes.

Shockwave Generation and Delivery: Utilize a shockwave generation module capable of producing shockwaves with the desired characteristics, including pulse duration, frequency range, and pressure amplitudes. For example, generate shockwaves with a pulse duration of 10 microseconds, a frequency range of 150 KHz-2 MHz, and pressure amplitudes between 100-150 MPa. Connect the shockwave generation module to the ultrasound therapy system for synchronized operation. Ensure proper calibration and alignment of the shockwave generation module with the target area to achieve optimal treatment outcomes.

Pulse Train Control: Determine the desired pulse repetition frequency (PRF) for the therapy based on the specific treatment goals and target tissue characteristics. For example, set the PRF to 5 Hz for a specific treatment session. Adjust the timing between consecutive shocks applied on the same spot to achieve the desired PRF. Use a timing control mechanism, such as a programmable timer or software algorithm, to regulate the timing intervals accurately. For example, set the time interval between shocks on the same spot to 200 milliseconds to achieve a PRF of 5 Hz. Continuously monitor and fine-tune the timing parameters during therapy to maintain the desired PRF.

Pulse Train Frequency: Determine the desired pulse train frequency (PTF) for the therapy based on the treatment objectives and the number of target spots. For example, set the PTF to 2 Hz for comprehensive coverage of all 25 target spots. Calculate the appropriate time interval between consecutive train of pulses to achieve the desired PTF. For example, set the time interval between consecutive train of pulses to 500 milliseconds to achieve a PTF of 2 Hz. Incorporate a pulse train frequency control mechanism, such as a control unit or software algorithm, to adjust and maintain the desired PTF throughout the therapy session. Continuously monitor the treatment progress and adjust the PTF if necessary to ensure comprehensive coverage of all target spots.

Regarding shockwave therapy parameters, it is important to note that certain treatment effects may share common physiological processes or target tissues, allowing for the utilization of overlapping shockwave parameters. This allows for the specification of a range of shockwave therapy parameters for a specific treatment effect, such as angiogenesis, without needing to individually specify parameters for each potential treatment effect the embodiments herein and their equivalents could enable.

For instance, angiogenesis and neovascularization are closely related processes involving the formation of new blood vessels. Based on existing research and clinical studies, shockwave therapy for angiogenesis has been shown to be effective within a range of shockwave parameters, including energy levels ranging from 0.01 to 0.1 mJ/mm², frequencies from 150 kHz to 2 MHz, and treatment durations of several weeks. These parameters have consistently demonstrated the ability to stimulate angiogenesis and promote blood vessel growth in various tissues.

In contrast, other treatment effects, such as reducing inflammation, promoting drug absorption in tissue, or neuromodulation-based heart arrhythmia therapy, may involve distinct physiological mechanisms or target different tissues. As a result, they may require specific shockwave parameters tailored to their unique requirements.

By specifying a range of shockwave therapy parameters for angiogenesis, some embodiments can provide a flexible and adaptable approach to promote angiogenesis across different tissues and patient populations. This allows for efficient utilization of the embodiment while reducing the need for extensive parameter variations for each potential treatment effect. It also enables healthcare professionals to optimize the therapy within the specified range based on individual patient characteristics and treatment goals.

In summary, the potential for overlapping shockwave parameters for certain treatment effects allows for the specification of a range of parameters specifically for angiogenesis. This approach provides versatility in treatment while streamlining the implementation and utilization across diverse applications. By focusing on angiogenesis as a representative treatment effect, such embodiments can effectively promote blood vessel growth and tissue repair in a wide range of clinical scenarios.

The adjustment of shockwave therapy parameters, whether increased or decreased, can be based on underlying physiological principles to optimize treatment outcomes. These principles guide the optimization of shockwave therapy parameters to achieve specific therapeutic effects. The following physiological principles are considered when adjusting the parameters:

Intensity and Energy Levels: The intensity and energy levels of shockwave therapy can be adjusted to modulate tissue penetration and mechanical stimulation. Higher intensity and energy levels may be required for deeper tissues or conditions requiring greater mechanical force, while lower levels may be suitable for superficial tissues or more sensitive areas.

Frequency: The frequency of shockwaves determines the compressions and rarefactions per unit of time. Lower frequencies provide deeper tissue penetration but may have a broader focus, while higher frequencies offer more localized effects with shallower penetration. The choice of frequency depends on the target tissue and desired therapeutic outcome.

Treatment Duration: The treatment duration refers to the total time of shockwave therapy administration. Longer durations may be necessary for conditions requiring a more significant therapeutic impact or for larger treatment areas. Shorter durations may suffice for smaller target areas or conditions necessitating more focused treatment.

Treatment Area: The treatment area defines the size and extent of the target tissue receiving shockwave therapy. Adjusting the treatment area involves determining boundaries and coverage based on the specific condition or target tissue. Larger treatment areas may require multiple applications or different scanning techniques to ensure sufficient coverage.

Frequency of Treatment: The frequency of shockwave therapy sessions, i.e., the frequency of treatment administration, may vary depending on the specific condition and treatment objectives. Some conditions may benefit from frequent sessions in close succession, while others may require longer intervals between sessions for optimal healing and recovery.

These physiological principles inform the adjustment of shockwave therapy parameters in accordance with specific patient and condition requirements. By tailoring the parameters to individual needs, the treatment can be optimized to achieve desired therapeutic effects while minimizing risks or adverse reactions.

Ultrasound therapy includes methods such as unfocused beaming, hyperthermia, high intensity focused ultrasound (HIFU), focused ultrasound, extracorporeal lithotripsy, extracorporeal shockwave therapy, phacoemulsification, ultrasound assisted liposuction, tissue cutting and vessel sealing, skin permeabilization, low intensity pulsed ultrasound.

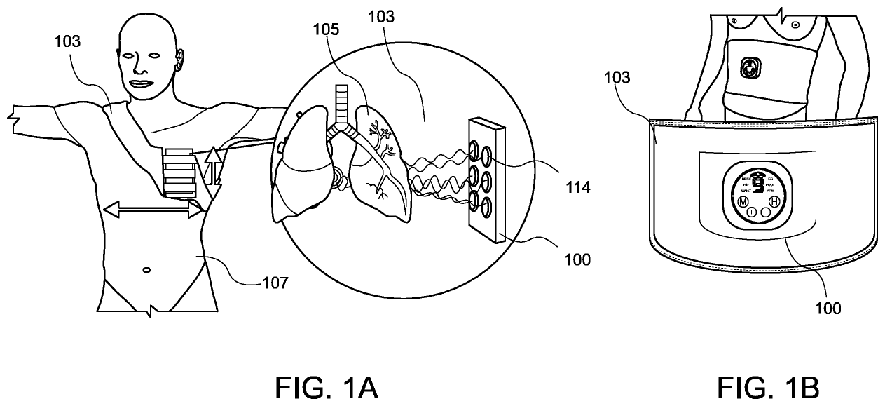

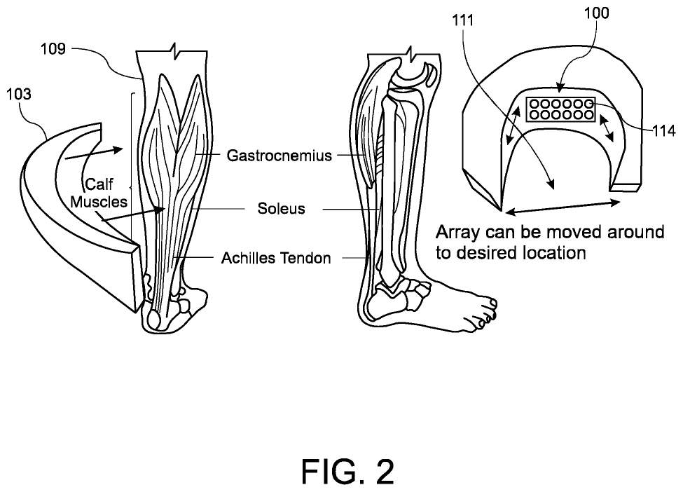

FIGS. 1A and 1B illustrate example embodiments of a wearable structure 103 as well as example embodiments of ultrasound therapy device 100. FIG. 1A shows a form factor of wearable structure 103 to secure the ultrasound therapy device 100 around the kidney region, or the lung region 105 of the user 107. FIG. 1B depicts another form factor of wearable structure 103 to secure the ultrasound therapy device 100 around the midsection of user 107 such as the stomach, diaphragm, or appendix. FIG. 2 illustrates a perspective view of the wearable structure 103 that allows the ultrasound therapy device 100 to be secured around the lower limb 109 of the user 107, in accordance with one embodiment of the present disclosure. FIG. 2 is explained in conjunction with FIGS. 1A and 1B.

The wearable structure 103 is securable to a user to hold the ultrasound therapy device 100 against the user's body to transmit the ultrasound to a target therapy area of a user including at least one of a kidney region, a lung region, and a lower limb of the user. The wearable structure 103 allows the ultrasound therapy device 100 to be “worn” or otherwise “adhered” to the user's body. In an embodiment, the wearable structure 103 can be in the form of a vest, or a belt/band that goes around the waist and/or around a lower limb region of the user (e.g., around the leg).

The wearable structure 103 includes a tightening mechanism 111 to adjust the tightness of the wearable structure 103 until it is snug around the chest, waist, and/or around the lower limb region of the user to prevent unnecessary movement of the ultrasound therapy device 100. In an embodiment, the tightening mechanism 111 includes but is not limited to a rubber tension or compression band with adjustable tightness, and an elastic strap with adjustable tightness. Additionally or alternatively, the tightening mechanism can include hook and loop fasteners, buttons, zippers, or other known garment/clothing/medical device technologies that can be used to secure the wearable structure 103 and the ultrasound therapy device 100 in the correct positioning.

The ultrasound therapy device 100 includes a grouping 119 of ultrasound transducer units 113. The ultrasound transducer units 113 are attachable and repositionable in wearable structure 103 to generate and deliver the ultrasound to the target region. The ultrasound transducer units 113 are arranged in an array. The array of ultrasound transducer units 113 is mechanically moved within the wearable structure 103 and is in contact with a material to facilitate penetration of ultrasound into the user's body.

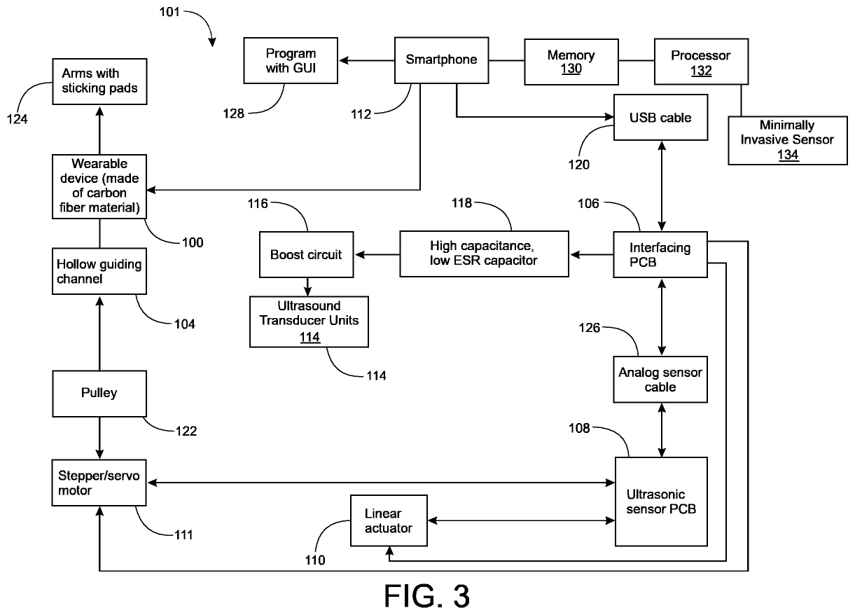

FIG. 3 illustrates a block diagram of example connections between the various components of the wearable structure configured to fit around one or more of: the kidney region, the lung region, and the lower limb of the user, in accordance with one embodiment of the present disclosure. FIG. 3 is explained in conjunction with FIGS. 1A and 1B and FIG. 2. The ultrasound therapy device 100 includes various ultrasound transducer units 114, a pressure sensor 133, a memory 130, and a processor 132. According to an embodiment herein, the ultrasound therapy device 100 includes a guiding channel 104, an ultrasonic sensor 108, a micro linear actuator 110, and a servo motor 111. The wearable structure 103 (as shown in FIGS. 1A and 1B and FIG. 2) is securable to the user to transmit the ultrasound to a target therapy area of a user including at least one of a kidney region, a lung region, and a lower limb of the user. In some embodiments, the ultrasound therapy device 100 acts as a gadget for the handheld computing device. Examples of the gadget include but are not limited to a casing, a cover, a housing, or an electrical housing. In some embodiments, the ultrasound therapy device 100, or at least the housing/body thereof, is made of carbon fiber material. Examples of handheld computing devices 112 include but are not limited to a computing device, smartphone, mobile device, phablet, tablet, etc. The ultrasound transducer units 114 are attachable and repositionable in the wearable structure 103 to generate and deliver the ultrasound to the target region. The ultrasound transducer units 114 are arranged in an array. The array of ultrasound transducer units 114 is mechanically moved within the wearable structure 103 and is in contact with an impedance matching material, gel, or fluid 300 such as ultrasound gel to facilitate penetration of ultrasound into the user's body. The pressure sensor 133 measures the proximity of the ultrasound transducer units 114 to the user's skin to determine adequate adhesion to the user's skin to facilitate ultrasound tissue penetration.

The ultrasound therapy device 100 is connected to at least one minimally invasive physiological sensor to collect the health status data of the user. In an embodiment, the minimally invasive physiological sensor is selected from the group of minimally invasive sensors 134.

The memory 130 includes ultrasound therapy instructions. The memory 130 is communicatively coupled to the processor 132. The memory 130 stores ultrasound therapy instructions executed by the processor 132. The memory 130 may be a non-volatile memory or a volatile memory. Examples of nonvolatile memory may include, but are not limited to flash memory, a Read Only Memory (ROM), a Programmable ROM (PROM), Erasable PROM (EPROM), and Electrically EPROM (EEPROM) memory. Examples of volatile memory may include but are not limited to Dynamic Random-Access Memory (DRAM), and Static Random-Access memory (SRAM).

The processor 132 processes the ultrasound therapy instructions. The processor 132 may include at least one data processor for executing program components for executing user- or system-generated requests. Processor 132 may include specialized processing units such as integrated system (bus) controllers, memory management control units, floating-point units, graphics processing units, digital signal processing units, etc. Processor 132 may include a microprocessor, such as AMD® ATHLON® microprocessor, DURON® microprocessor OR OPTERON® microprocessor, ARM's application, embedded or secure processors, IBM® POWERPC®, INTEL'S CORE® processor, ITANIUM® processor, XEON® processor, CELERON® processor or other line of processors, etc. Processor 132 may be implemented using a mainframe, distributed processor, multi-core, parallel, grid, or other architectures. Some embodiments may utilize embedded technologies like application-specific integrated circuits (ASICs), digital signal processors (DSPs), Field Programmable Gate Arrays (FPGAs), etc.

Processor 132 may be disposed of in communication with one or more input/output (I/O) devices via an I/O interface. I/O interface may employ communication protocols/methods such as, without limitation, audio, analog, digital, RCA, stereo, IEEE-1394, serial bus, universal serial bus (USB), infrared, PS/2, BNC, coaxial, component, composite, digital visual interface (DVI), high-definition multimedia interface (HDMI), RF antennas, S-Video, VGA, IEEE 802.n/b/g/n/x, Bluetooth, cellular (e.g., code-division multiple access (CDMA), high-speed packet access (HSPA+), global system for mobile communications (GSM), long-term evolution (LTE), WiMAX, or the like), etc.

The processor 132 is configured to transmit the health status data to a server 129 over a network. Network may be a wired or a wireless network, and the examples may include but are not limited to the Internet, Wireless Local Area Network (WLAN), Wi-Fi, Long Term Evolution (LTE), Worldwide Interoperability for Microwave Access (WiMAX), and General Packet Radio Service (GPRS).

The server 129 is configured to analyze the collected health status data to obtain the detected results and transmit them to the computing device over the network for a presentation. Various examples discussed herein refer to one or more servers. As used herein, a “server” is any server computer capable of performing functions stored in a computer-readable storage medium of the server computer. The server computer may download the program code to the processor for use on the computing devices, client computers, or electronic devices.

The ultrasound therapy instructions comprising: determining a non-acute health status of the user based on health status data of the user received by the ultrasound therapy device 100 from a minimally invasive physiological sensor; receiving ultrasonic data from the ultrasound transducer units 114; determining a location of the array of ultrasound transducer units 114 on the user's body; transmitting ultrasound therapy information to the one or more ultrasound transducer units 114; and generating ultrasound therapy in a target therapy area of the user, after a non-acute health state of the user and correct location of the array has been established. In an embodiment, the ultrasound therapy information includes at least one of frequency, temporal average, duty cycle, and therapy duration. In an embodiment, the ultrasound therapy information is configured to induce pulmonary rehabilitative effects. In an embodiment, the ultrasound therapy information is configured to promote one or more of angiogenesis, reduce inflammation, improve renal fibrosis and tubular injury, neovascularization, and ameliorating inflammatory processes. In an embodiment, the ultrasound therapy information includes but is not limited to location data, frequency data, spatial average temporal average data, duty cycle data, and therapy duration data.

In an embodiment, the ultrasound therapy device 100 includes a minimally invasive sensor 134 for minimally invasively acquiring one or more of: pulmonary function information, renal function information, peripheral arterial function, and deep vein thrombosis (DVT) function information of the user.

In an embodiment, the vibration sensor is facing away from the user's skin to enable the detection of environmental acoustic disturbances that could interfere with receiving and sending signals.

In some aspects, the techniques described herein relate to an ultrasound therapy device, wherein a second vibration sensor is facing away from the skin-facing side and the processor is configured to enable detection and removal of surrounding acoustic disturbances sensed by the second vibration sensor with receiving and sending signals. A vibration sensor includes but is not limited to any sensor that detects and measures mechanical oscillations or vibrations in different mediums, including air, solid bodies, and fluids. It converts these vibrations into electrical signals for analysis. The sensor can be based on principles such as piezoelectric, capacitive, or piezoresistive effects for solid bodies and hydrodynamic or piezoelectric effects for fluids.

In an embodiment, the pulmonary function information comprises one or more minimally invasive data from the lungs and self-reported pulmonary health data. In an embodiment, the renal function information comprises one or more minimally invasive data from the kidney and self-reported kidney health data. In an embodiment, the deep vein thrombosis (DVT) function information comprises one or more minimally invasive data from the lower limb and self-reported lower limb health data.

In an embodiment, the processor 132 is configured to receive pulmonary function information, and transmit ultrasound therapy information to the one or more ultrasound transducer units 114 for generating ultrasound therapy. In an embodiment, the processor 132 is configured to receive renal function information, and transmit ultrasound therapy information to the one or more ultrasound transducer units 114 for generating ultrasound therapy. In an embodiment, the processor 132 is configured to receive deep vein thrombosis (DVT) function information, and transmit ultrasound therapy information to the one or more ultrasound transducer units 114 for generating ultrasound therapy.

In an embodiment, the ultrasound therapy is administered in one or more patients diagnosed with one or more of bronchitis, Chronic Obstructive Pulmonary Disease, Cystic Fibrosis, Emphysema, Idiopathic Pulmonary Fibrosis, flu, lung cancer, obstructive sleep apnea, pleurisy, Tuberculosis, pulmonary congestion, kidney diseases, and peripheral artery disease.

In an embodiment, the ultrasound therapy device 100 includes a circuit board (PCB) 106 to connect the one or more ultrasound transducer units 114, the proximity sensor(s), the minimally invasive sensor(s) 134, the positioning mechanism, the processor 132, or pressure sensors. In an embodiment, the proximity sensor is a pressure sensor for measuring the pressure that the one or more ultrasound transducer units 114 apply on the user's skin. The pressure sensors are configured to measure the proximity of an ultrasound transducer to the user's skin. The pressure sensors may include any instruments or devices that translate the magnitude of a physical pressure being exerted onto the sensor into an output signal that can be used to establish a quantitative value for the pressure. Pressure sensors may include but are not limited to potentiometric pressure sensors, inductive pressure sensors, capacitive pressure sensors, piezoelectric pressure sensors, strain gauge pressure sensors, variable reluctance pressure sensors, aneroid barometer sensors, Manometer Sensors, Bourdon Tube Pressure Sensors, Vacuum Pressure Sensors, Sealed Pressure Sensors.

In an embodiment, the ultrasound therapy device 100 includes an inertial measurement unit (IMU) sensor 115 configured to determine whether the ultrasound therapy device 100 is correctly positioned on the user's skin and/or determines user movement.

In an embodiment, the PCB 106 further includes the ultrasonic sensor 108 which is connected to the circuit board (PCB) 106 via an analog sensor cable 126. In an embodiment, the ultrasonic sensor 108 includes a MEMS ultrasonic sensor. In an embodiment, the ultrasonic sensor 108 is a piezoelectric ultrasonic sensor. The PCB 106 can refer to a printed wiring board, printed wiring card, or a printed circuit board (PCB) that mechanically supports and electrically connects electrical or electronic components of the presently disclosed device using conductive tracks, pads, and other features etched from one or more sheet layers of copper laminated onto and/or between sheet layers of a non-conductive substrate.

In an embodiment, the ultrasound therapy device 100 includes a housing for accommodating at least a part of one or more ultrasound transducer units 114, the proximity sensor, and the processor 132.

In an embodiment, the processor 132 is configured to analyze one or more of the pulmonary function information, the renal function information, and/or the deep vein thrombosis (DVT) function of one or more of the lung, the kidney, and the lower limb of the user and determine one or more locations on the lung the kidney and the lower limb of the user where the one or more of the ultrasound signals are focused based on ultrasonic sensor data of the one or more ultrasound transducer units 114.

In an embodiment, the circuit board is a PCB 106 is connected to one or more of: an analog-to-digital converter (ADC) for converting analog ultrasonic data into digital data; a micro-controlling unit with a power and data transmission port; one or more large bandwidth operational amplifiers circuits; a plurality of digital buffers; at least two signal mixers for precise doppler calculation; a plurality of filters suitable for an operating range of a piezoelectric ultrasonic sensor; a plurality of bidirectional drivers for a micro linear actuator and a servo motor; and a plurality of headers and a plurality of PWM lines to provide the power to the micro linear actuator and the servo motor.

In an embodiment, the ultrasound therapy device 100 includes a boost circuit 116 for providing power feed to the one or more ultrasound transducer units 114. In an embodiment, the boost circuit 116 comprises a low equivalent series resistance (ESR) capacitor and utilizes an accumulated charge on high capacitance.

In an embodiment, the array is configured to direct the ultrasound beam to the one or more regions of the lung, the kidney, and the lower limb and execute the ultrasound therapy information in the one or more regions.

In an embodiment, the ultrasound therapy device 100 is configured to compare the pulmonary or renal health of the user in the one or more regions over time to determine the efficacy of the ultrasound therapy. In an embodiment, the ultrasound therapy device 100 is configured to update the ultrasound therapy based on the observed efficacy of the ultrasound therapy over time. In an embodiment, the ultrasound therapy device 100 is configured to create a map of the user's lungs, kidneys, and lower limbs. In an embodiment, the ultrasound therapy device 100 is configured to collect information on the user's health condition through a questionnaire and/or patient health database.

In an embodiment, the ultrasound therapy device 100 includes a pressure adjustment mechanism configured to adjust the tightness of the ultrasound transducer units 114 to the user's skin. In an embodiment, the ultrasound therapy device 100 receives power through a cable from an external electric power system.

Often used with the device is an impedance matching material, gel, or fluid 300 to facilitate the ultrasound therapy device's contact with the skin, help steer the ultrasound to the target therapy area and prevent dispersion or other loss of the waves. In an embodiment, the ultrasound therapy device 100 includes impedance matching material, gel, or fluid 300 or is used with a removable ultrasound gel patch that is connected to the skin-facing side of the ultrasound therapy device 100. In another embodiment, the ultrasound therapy device 100 is used with ultrasound gel.

The ultrasound transducer units 114 are attached to the PCB 106 via a spring-based mechanism to generate ultrasounds. The pressure sensors measure the proximity of the ultrasound transducer units 114 and/or the ultrasonic sensor 108 to the skin. The ultrasonic sensor 108 is configured to detect different regions of the user's organ and determine a location on the chest where the ultrasound transducer units 114 are placed. The micro linear actuator 110 is attached to the ultrasonic sensor 108 to acquire Pulse Width Modulation (PWM) control from the interfacing circuit board 106. The micro linear actuator 110 is configured to place the ultrasonic sensor 108 on the skin of a user to obtain analog data with a sticking force chosen to minimize an error resulting from pressing the skin with the ultrasonic sensor 108.

In some embodiments, the ultrasound transducer units 113 operate with a spring-ball detent system or the equivalent. This helps the ultrasound therapy device 100 push the individual ultrasound transducers against the skin especially if the skin is not flat. The system is optionally designed to prevent lateral movement of the ultrasound transducer units.

The servo motor 111 is attached to the micro linear actuator 110 to acquire the PWM control from the interfacing circuit board 106 via a bidirectional PWM driver on the interfacing circuit board 106. In some embodiments, the micro linear actuator 110 includes a static side and a moving stroke and is attached to the servo motor 111 from the static side, and the ultrasonic sensor 108 is attached to the moving stroke of the micro linear actuator 110. In some embodiments, the pressure sensors, the ultrasound transducer 114, and the piezoelectric ultrasonic sensor 108 is soldered to slim printed circuit board (PCB) 106.

In some embodiments, the servo motor 111 moves in a plurality of channels created in the body of the ultrasound therapy device 100. In some embodiments, functions of the servo motor 111 may be performed by a stepper motor. The servo motor 111 is powered and controlled by the interfacing circuit board (PCB) 106. In some embodiments, the servo motor 111 is attached to the micro linear actuator 110 to acquire the PWM control from the interfacing circuit board 106 via a bidirectional PWM driver placed on the interfacing circuit board 106. In some embodiments, the hollow guiding channel 104 is built into the body of the ultrasound therapy device 100 to guide and restrict the movement of the servo motor 111.

In some embodiments, the interfacing circuit board (PCB) 106 is connected to the handheld computing device 112 via a power and data transmission cable 120 with data and power lines. The power and data transmission cable 120 receives power from the handheld computing device 112.

In some embodiments, the ultrasound transducer 114 obtains power feed from a boost circuit 116. In some embodiments, the boost circuit 116 utilizes an accumulated charge on high capacitance and a low equivalent series resistance (ESR) capacitor 118. The accumulated charge is acquired from the handheld computing device 112 via the power and data transmission cable 120 during idle time.

In some embodiments, ultrasound therapy device 100 includes a pulley 122 attached to the servo motor 111 to move the ultrasound transducer unit inside the hollow guiding channel 104 in the body of the ultrasound therapy device 100. In some embodiments, the ultrasound therapy device 100 includes a plurality of arms with fixation pads 124 are attached to the bottom of the housing 102 allowing the gadget or device 100 to be attached to the user's skin.

In some embodiments, the ultrasound therapy device 100 is powered by the handheld computing device 112 or may obtain power from an external battery that can supply electrical power to the PCB 106. The external battery can be non-rechargeable or rechargeable (e.g. alkaline batteries, zinc-carbon batteries vs Lithium-ion batteries, Nickel-Metal hydride batteries, Lithium Polymer batteries). The external battery can be based on Lithium Polymer (Li-Poly) and Lithium-Ion (Li-Ion). Furthermore, the battery can be operated by a power management integrated circuit such as power MOSFETs. Alternatively, the ultrasound therapy device 100 can be powered by a handheld computing device. In one embodiment, the ultrasound therapy device 100 can be powered by a power source, where the power source can be one or batteries, AC mains, an inductive power transfer with no physical contact, where the inductive power transfer is powered by e.g. AC mains, fiber optic power supply, where the fiber optic power supply can e.g. comprise an optical-to-electrical converter, like a solar cell, for providing electrical energy, a solar array for providing electrical energy, a wind-up manual system for providing electrical energy e.g. by an electrical generator driven by the wind-up manual system, or an energy-harvesting system for providing electrical energy from a magnetic field. In the embodiment where inductive power transfer relies upon an AC main, the ultrasound therapy device 100 may comprise a filter to shunt power transients of high frequency and/or large magnitude.

To avoid any measurement error caused by the weight of the measurement device, a hollow housing made from carbon fibers similar to that of the handheld computing device can be used to create a hollow space around the sensor to guarantee there is no extra weight from the ultrasound therapy device 100 on the skin between the sensor and artery, and thus guarantee precise results, so the pressure applied to the skin by the ultrasound therapy device 100 is exerted at points far from the artery and has no effect on the artery diameter or shape.

A great advantage of employing handheld computing device technologies is to provide cheap and reliable access to pulmonary function, renal function, peripheral arterial function, and deep vein thrombosis (DVT) function measurement, and add the feature of an ultrasound to harness the thermal and non-thermal effects of high-intensity focused ultrasound (HIFU) and ultrasounds to treat organ failure, for relief and mobility restoration. The ultrasound therapy device 100 can work with automated precision sensor positioning and negligible error of the sensor weight.

The individual transducer units 114 can be phase shifted with respect to each other. Depending on the relative phase shift between the transducer units, the position of the maximum of the total superposition wave at a plane with respect to the 1D or 2D array can be positioned by adjusting the phases for each of the transducer units in the array. Using this method, the ultrasound can be steered to a specific angle (and position) without using any moving mechanical parts. Likewise, the ultrasound waves from the transducer units can be focused at a certain area of the organ, and the ultrasound can be controlled regarding angle and focus on the organ. In an embodiment, the array can be one-dimensional or two-dimensional. If the array is one-dimensional, the generated ultrasounds can be controlled in one dimension. If the array is two-dimensional, the generated ultrasounds can be controlled in two dimensions. In an embodiment, the at least one array of ultrasound transducer units can be configured to generate electric signals as responses to reflected ultrasound waves from the kidneys, lungs, and limbs. That the transducer units can be two-way transducers means that the array of ultrasound transducer units can generate in addition to the ultrasounds for treating kidney disease, peripheral artery disease, and deep vein thrombosis also generate ultrasound waves and register the reflected ultrasound waves for generating a picture of the kidneys, lungs, and limbs and even a moving picture of the kidneys, lungs, and limbs.

When the reflected ultrasound waves enter the array of ultrasound transducer units the reflected ultrasound waves create voltage/current responses from the ultrasound transducer units depending on the strength of the amplitude of the vibrations/pressure wave. This means that the array of ultrasound transducer units can be used as an active sensor array for monitoring the status of the organ. Since the reflected ultrasound waves will reach the different transducer units at different times, the array of ultrasound transducer units can register the phase difference and build a picture of the organ based on the reflected ultrasound waves. The array of ultrasound transducer units can be used to localize the organ in the extrapolated actuator/sensor array plane by using the amplitude and time delay between each sensor/actuator in the array.

In general, a controller sends electrical signals for controlling the ultrasound transducer units, where the electrical signals are converted to ultrasound waves. Phased array systems are composed of an array of transducer units in 1D or 2D arrays and can transmit waves independently at different times or phase changes between the transducer units. To focus or to steer the ultrasound waves, time delays or phase changes are applied to the ultrasound transducer units to create constructive interference of the wavefronts of the ultrasound wave from each ultrasound transducer unit. Due to this effect, the ultrasound wave can be steered to a certain angle, and/or the energy can be focused on any position of the kidneys, lungs, and limbs.

After transmitting the ultrasound waves, the ultrasound transducer units receive the reflected ultrasound waves from the organ as an echo, as well. By having the ultrasound transducer units to be used inversely, the ultrasound transducer units can convert the received reflected ultrasound waves into electrical signals that can be registered by the controller. In the same way, the array of ultrasound transducer units can steer and focus the generated ultrasound waves as mentioned above, and the array of ultrasound transducer units can determine the direction and origin of the reflected ultrasound waves. All received waves are converted to electrical signals and can be evaluated by signal analysis to obtain the health status of the kidneys, lungs, and limbs and/or the effectiveness of the transmitted ultrasound to the kidneys, lungs, and limbs.

The advantages of phased array systems like the array of ultrasound transducer units include the ability to perform scanning of the ultrasound waves, which reduces inspection times by eliminating or reducing the need to mechanically move the array of ultrasound transducer units.

When the array of ultrasound transducer units 114 is used for generating an image of the organ the energy level of the generated ultrasound waves may be lower or much lower than 1 W/cm².

Since the ultrasound waves have a frequency above 20 kHz, the acoustic transducer, like a microphone or an accelerophone, only sensitive to sound waves below 20 kHz or below 15 kHz will not be able to register the ultrasound.

The vibration sensor can preferably be sensitive within the range of 3-20 kHz, preferably within the range of 4-20 kHz, and most preferably within the range of 5-20 kHz.

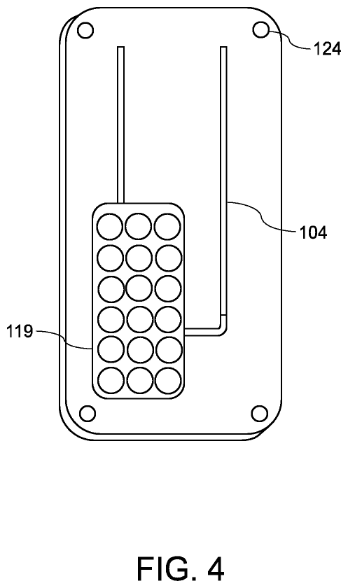

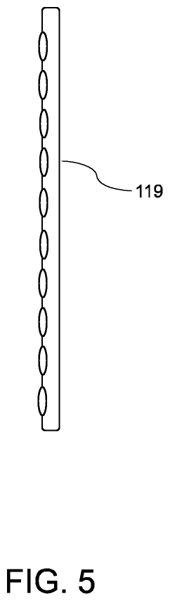

FIG. 4 illustrates a bottom view of the ultrasound therapy device 100, in accordance with one embodiment of the present disclosure. FIG. 4 is explained in conjunction with FIG. 3. The arms with fixation pads 124 are configured to be placed on the body of the patient. The hollow guiding channel 104 is placed into the body of the ultrasound therapy device 100 to guide the ultrasound transducer unit 114 via movement of the stepper motor or servo motor 111 and the linear actuator 110 and PCB 106 of the ultrasonic sensor 108 attached to it. According to an embodiment herein, the guiding channel 104 is placed into the body of the ultrasound therapy device 100. The guiding channel 104 is configured to position the soundwave transducer unit within a target therapy area of the user. The PCB of the ultrasonic sensor 108 is attached to the stepper motor 111 via the linear actuator 110. FIG. 5 illustrates a side view of the ultrasound therapy device 100, in accordance with one embodiment of the present disclosure.

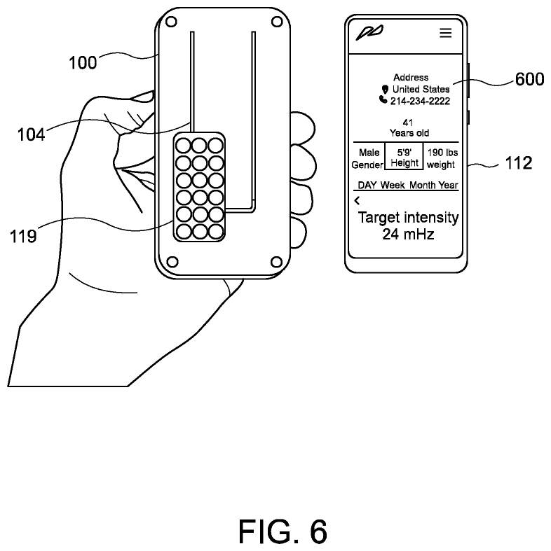

FIG. 6 illustrates a monitoring application 600 installed within the handheld computing device 112, in accordance with one embodiment of the present disclosure. FIG. 6 is explained in conjunction with FIG. 3. The monitoring application 600 may be based on one or more operating systems comprising Android®, and iOS®. The ultrasound therapy device 100 requires the user to register on the monitoring application 600 installed or configured within the handheld computing device 112. Memory 130 is configured to register the user over the monitoring application 600 by receiving one or more credentials from the user for providing access to the monitoring application 600.

Examples of the credentials include but are not limited to a username, password, age, gender, phone number, email address, location, etc. In some embodiments, the monitoring application 400 is commercialized as a software application or a mobile application, or a web application for health assessment. A user may include a patient, a patient using the monitoring application using the handheld computing device 112. In some embodiments, the monitoring application 600 is a combination of a software program with a graphical user interface (GUI) 205 (shown in FIG. 6) which is running on the handheld computing device 112 to present resulting data such as name, location, age, gender, height, weight, periodical target intensity, etc. and allow the user to do suitable adjustments based on the resulting data. The resulting data is obtained by one or more ultrasonic sensors 108 configured with the ultrasound therapy device 100.

According to an embodiment herein, processor 132 processes the captured/obtained data and transmits it to an external computing device or a server 129 for further processing over a network. The processed data related to the organ's health of the user is presented on the monitoring application 400. The network may be a wired or a wireless network, and the examples may include but are not limited to the Internet, Wireless Local Area Network (WLAN), Wi-Fi, Long Term Evolution (LTE), Worldwide Interoperability for Microwave Access (WiMAX), and General Packet Radio Service (GPRS).

The monitoring application 600 enables the user to continuously monitor the organ function. Further, the monitoring application 600 utilizes machine learning for automatic positioning and determining the intensity of ultrasonic transducers.

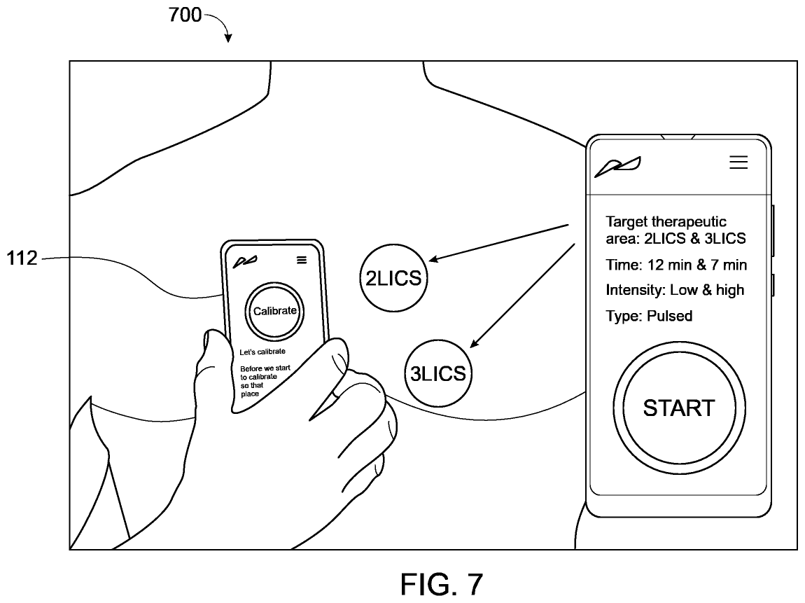

FIG. 7 illustrates a perspective view 700 of the handheld computing device placed against the body or chest of a user, in accordance with at least one embodiment. FIG. 7 is explained in conjunction with FIG. 6. The monitoring application 600 directs the user through the GUI 205 to start the measurement of organ function. Then the user places the handheld computing device against his/her chest as shown in FIG. 6. The handheld computing device 112 may have a shape adapted to fit firmly on the target therapy area. The shape of the handheld computing device 112 is a bend or curved so that it perfectly fits on the patient's target therapy area.

In some embodiments, the ultrasonic sensor 108 is combined with the one or more proximity sensors, such as pressure sensors, that allow the linear actuator to place the ultrasound transducer unit 114 and/or the ultrasonic sensor 108 right on the skin with minimum and fixed sticking force to minimize the error resulting from pressing the skin with a probe. According to an embodiment herein, the ultrasound therapy device 100 utilizes a closed-loop control using a digital PID algorithm to ensure that the applied force to the skin doesn't cause additional errors in the measurement process. Further, ultrasound therapy device 100 utilizes the closed-loop control using the digital PID algorithm to ensure that the position of the sensor is optimized automatically to make sure that the applied measurement cannot be optimized further.

In some embodiments, the ultrasonic sensor 108 uses an integrator as a part of ultrasonic calculator circuits. This can be a solution where sampling of the direct output waveform can introduce many problems since the output waveform does not have an exact wave shape, while an integrator allows measurement of the changes by determining the nonlinearity of the resulting waveform from the integrator. Finally, the optional use of analog filters helps to ensure that any noise from external sources is neglected so that the input of the integrator is known to be from the ultrasonic reading rather than ambient EM waves at the integrator input.

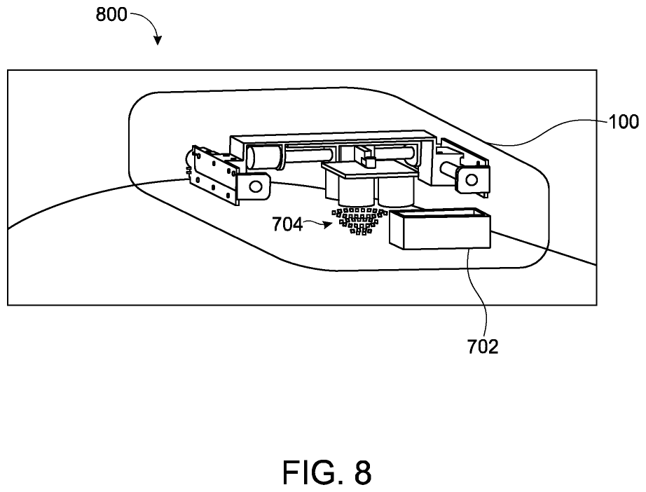

FIG. 8 illustrates a first perspective view 800 of the placement of the ultrasound therapy device 100 (transparent) against the body of the user, in accordance with at least one embodiment. According to an embodiment herein, the ultrasound therapy device 100 includes a container 702 to store impedance matching material, gel, or fluid 300. FIG. 8 also depicts an example ultrasound wave 704 generated by the ultrasound transducer units 114.

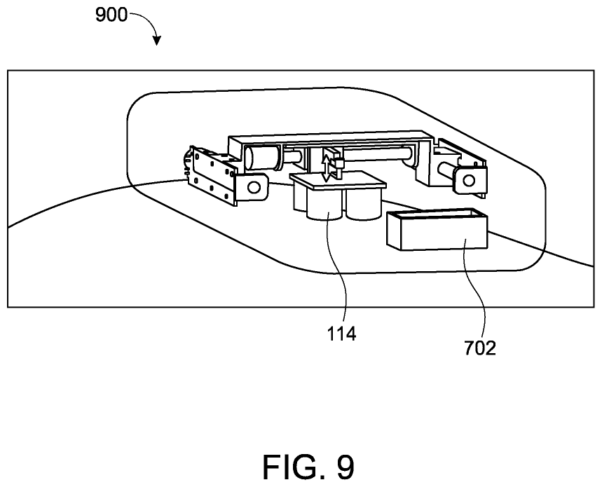

FIG. 9 illustrates a second perspective view 900 of the placement of the ultrasound therapy device 100 (transparent) against the chest of the user, in accordance with at least one embodiment. FIG. 9 is explained in conjunction with FIG. 8. The pulsation frequency and strength of the ultrasound generated by the ultrasound transducer units 114 are directed at suppressing hypertrophic cardiomyopathy and/or myocardial interstitial fibrosis.

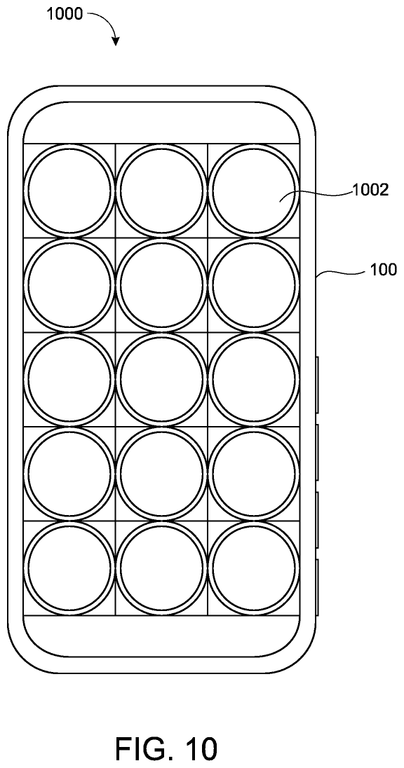

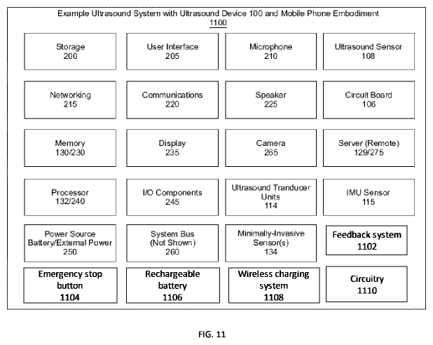

FIG. 10 illustrates a skin-facing side view of an example embodiment of ultrasound therapy device 1000 having a matrix or array of ultrasound transducer units. FIG. 10 is explained in conjunction with FIG. 3. In another embodiment, the ultrasound therapy device 100 of the present ultrasound therapy device 100 includes a matrix of ultrasound transducers 1002 instead of the linear actuators and a guiding channel. According to an embodiment herein, different of the ultrasound transducers 1002 are activated depending on which region of the organ needs treatment. In an embodiment, the ultrasound transducers 114 are activated in accordance with the region of the organ that is to be analyzed and/or treated. The ultrasound transducer units 114 can be adjusted electronically and/or mechanically to ensure a good fit with the user's skin.

In one embodiment, the array of transducer units can be configured to generate ultrasound signals or pulsations for ultrasound therapy and in addition be configured for providing an electrical signal in response to an incoming ultrasound signal or pulse.

FIG. 11 depicts an example electronics diagram of an example ultrasound system 1100. ultrasound system 1100 includes ultrasound therapy device 100 and a mobile phone (or other handheld device), which lets the ultrasound therapy device have more functionality while using the resources that a user might already have on them. The example mobile phone contains storage 200, User Interface (e.g., a touch screen) 205, Microphone 210, Networking components 215, telephony components 220, speaker 225, Memory 230, touch screen display 235, processor 240, Input/Output (I/O) Components 245, Battery/Power System 250, Camera 255, and System Bus 260 connecting the various components together. The example ultrasound therapy device contains circuit board 106, vibration sensor 108, ultrasound transducers 114, memory 130, processor 132, minimally invasive sensors 134, ultrasound transducers 114, an optional IMU sensor 115, The listed elements can be on either or both of the ultrasound therapy device or the mobile phone. (This is why some elements are shown with more than one reference numeral.) For example, the processor and memory may be only on the mobile phone and the ultrasound therapy device can take the form of a mobile phone case. Alternatively, the ultrasound therapy device can be integral with the mobile phone. The system with similar components can take other forms and be without a mobile phone. The power source can use batteries and/or external power sources and be on one or both devices. The electrical systems, if needed, could be modified to include the ability to transmit and/or draw significant amounts of power. A server 129/275 could be on one of these devices. Alternatively, the server 129/275 can be remote so that complex computing and calculations could be performed at a location with higher performance capabilities which helps keep the ultrasound therapy device and mobile phone simpler and more economical.

Alternatively, the mobile phone here could be another handheld device like a tablet, laptop, or a smartwatch. Additionally or alternatively, it could be separate from or integrated with the ultrasound therapy device.

According to some embodiments herein, a device 100 generates and delivers ultrasound treatment to a target therapy area of a user. In an embodiment, the device 100 is an ultrasound therapy device. The device 100 includes a housing 102, an array of ultrasound transducer units 114, a user interface 205, a memory 130, and a processor 132. The array of ultrasound transducer units 114 is placed on the skin-facing side of the housing 102. The array of ultrasound transducer units 114 generates and delivers ultrasound therapy to a target therapy area of a user. The user interface 205 receives input information from the user about the target therapy area. The input information comprises the size and shape of the target therapy area. The memory 130 stores a plurality of ultrasound therapy instructions. The processor 132 processes the ultrasound therapy instructions. The ultrasound therapy instructions comprising: determining the location and the depth of the target therapy area based on the input information; calculating an optimal configuration for the array of ultrasound transducer units 114 based on the location, depth, size, and shape of the target therapy area; transmitting ultrasound therapy information to the ultrasound transducer units 114; generating an ultrasound therapy by the array of ultrasound transducer units in the target therapy area of the user based on the optimal configuration of the ultrasound transducer units 114; sending and receiving ultrasonic waves from the ultrasound transducer units 114 to test an expected throughput of the impedance matching material, gel or fluid, and skin contact; determining if expected throughput is allowable based on a pre-existing threshold; and instructing the processor 132 to start ultrasound therapy if the expected throughput is allowable.

In an embodiment, the device 100 includes a feedback system 1102 that is configured to receive feedback from one or more of the user, and a plurality of physiological sensors to adjust the ultrasound therapy as needed during treatment.

In an embodiment, the device 100 includes a circuitry 1110 that is configured to automatically shut off the device upon detection of one or more of excessive heat, pain, and an indicator of potential harm. Potential harm indicators may include but are not limited to excessive pressure, tension or strain exerted on the body measured by incorporated sensors, biometric feedback given by physiological sensors incorporated in the device capable of monitoring vital signs and able to show abnormalities that could show potential distress, or user feedback by incorporating buttons or controls that allow the user to provide feedback about their comfort level or any perceived harm.

In an embodiment, the device 100 includes an emergency stop button 1104 that is configured to allow the user to terminate the ultrasound therapy as and when required.

In an embodiment, the device 100 includes one or more of a rechargeable battery 1106 and a wireless charging system 1108 that are configured to charge one or more components of the device and reduce the need for constant power source replacement.

In an embodiment, the target therapy area includes but is not limited to a kidney region, a lung region, a lower limb region, a neck region, a shoulder region, an upper limb region, an abdominal region, and a back region of the user.

In an embodiment, the array of ultrasound transducer units 114 is configured to operate directly through the skin of the user near the target therapy area or indirectly through one or more of the impedance-matching material, gel, and fluid near the target therapy area.

In an embodiment, the ultrasound therapy device takes the form of a wearable patch configured to induce cardioprotective effects during an acute condition of the patient. The wearable patch may be configured to be worn on the patient's chest. In an embodiment, the acute condition may include but is not limited to acute myocardial infarction and cardiogenic shock.

In an embodiment, the ultrasound therapy device is configured to randomly administer ultrasound therapy within a certain predefined area. The administration of said ultrasound therapy will ensure that ultrasound will get through to the desired area in the desired format. The ultrasound therapy parameters may be configured to be harmless to certain parts of the body. In such an embodiment, bone tissue may for example simply absorb the ultrasound therapy without any effect, whereas other human organ tissue may be affected by said therapy.

In an embodiment, the ultrasound therapy device is configured to detect the presence of bone. For example, if a bone that may represent the rib cage is detected, then the ultrasound therapy will be adjusted to a position where there are no bones (or other things) blocking the therapy. The bone may be detected by comparing an expected reflection of ultrasound with that of the actual reflection observed. The thinking here is that bone will cause a faster reflection of ultrasound.

In an embodiment, the feedback system 1102 monitors the effectiveness of the therapy in real time. The feedback system 1102 may include additional sensors such as temperature sensors, motion sensors, or pressure sensors) that collect data during the therapy session and adjust the ultrasound intensity, frequency, or duration accordingly.

In an embodiment, the user interface 205 allows the users to easily select the target therapy area, control the intensity of the ultrasound therapy, and monitor their progress during the therapy session. This can be achieved by incorporating a touchscreen display or a mobile app that connects to the device via Bluetooth or Wi-Fi.

In an embodiment, the user interface 205 allows the users to create personalized therapy profiles based on their medical history, preferences, and requirements. The processor can use this information to adjust the ultrasound therapy settings for optimal results. This may also include a machine learning algorithm that can adapt the therapy settings based on user feedback or historical data.

Further, the device incorporates various safety features such as automatic shut-off when the device detects excessive heat, pain, or other indicators of potential harm. Potential harm indicators may include but are not limited to excessive pressure, tension or strain exerted on the body measured by incorporated sensors, biometric feedback given by physiological sensors incorporated in the device capable of monitoring vital signs and able to show abnormalities that could show potential distress, or user feedback by incorporating buttons or controls that allow the user to provide feedback about their comfort level or any perceived harm.

In an embodiment, the device uses a rechargeable battery 1106 or a wireless charging system 1108 to improve the device's portability and ease of use. This will allow users to easily charge the device and reduce the need for constant power source replacement.

Further, the present device enhances the comfort and fit of the wearable structure by using adjustable straps or an adaptable design that conforms to different body shapes and sizes. This can be achieved by using lightweight, flexible, and breathable materials that are hypoallergenic and easy to clean.

According to an embodiment herein, the present device integrates a dynamic ultrasound focusing system that adapts to the user's body shape and size. By using adaptive algorithms, the device can focus the ultrasound energy specifically on the target therapy area, optimizing the therapy's efficiency while minimizing the impact on surrounding tissues.

According to an embodiment herein, the present device implements an artificial intelligence (AI) system that analyzes a user's unique physiological characteristics, medical history, and response to previous therapy sessions to generate a personalized therapy plan. The AI-driven therapy could continually adapt and optimize the treatment parameters based on real-time or near real-time feedback, significantly improving the overall effectiveness and safety of the therapy.

According to an embodiment herein, the present device incorporates a mechanism for ultrasound-triggered drug delivery, where microcapsules or nanoparticles containing medication are injected into the body and selectively released at the target therapy area upon exposure to the ultrasound waves. This would enable localized and controlled drug delivery, potentially enhancing the therapeutic outcomes while reducing systemic side effects.

According to an embodiment herein, the present device combines the ultrasound therapy device with other therapeutic modalities, such as electrical stimulation, light therapy, or vibration therapy, to create a synergistic effect. The device could deliver these therapies either simultaneously or sequentially, depending on the user's needs and therapy plan, potentially increasing the overall effectiveness of the treatment.

According to an embodiment herein, the present device integrates a haptic feedback system into the wearable structure to guide users during the self-administration of the therapy. The system could use vibrations or other tactile sensations to indicate the optimal positioning of the device or adjustments required in real-time or near real-time, ensuring accurate and effective therapy delivery.

According to an embodiment herein, the present device utilizes an AR-based guidance system that uses a smartphone or wearable AR device to provide real-time or near real-time visual instructions to users on how to position and use the ultrasound therapy device correctly. The AR system could also display progress, therapy settings, and safety information during the treatment.

According to an embodiment herein, the present device enables remote monitoring and control of the ultrasound therapy device by healthcare professionals, allowing them to observe the therapy session, adjust settings, or provide guidance in real time. This feature could enhance the safety and effectiveness of the therapy while allowing users to receive professional assistance from the comfort of their own homes.

In an embodiment, the dynamic ultrasound focusing system is designed to adaptively focus ultrasound energy on a target therapy area, taking into account the user's body shape, size, and tissue properties. This system ensures that the ultrasound waves are concentrated specifically on the target area, optimizing therapy efficiency while minimizing the impact on surrounding tissues.

In implementation, the dynamic focusing system consists of an array of ultrasound transducer units that can adjust their positions and orientations relative to each other. The transducers could be mounted on a flexible, stretchable, or otherwise adjustable substrate, which would allow them to conform to the user's body surface and maintain optimal contact during therapy.

In an embodiment, the dynamic focusing system incorporates real-time or near real-time ultrasound imaging to visualize the target therapy area and surrounding tissues. This would provide the necessary information to adjust the focal point and ultrasound beam characteristics, ensuring that the therapy is accurately targeted.

In an embodiment, the dynamic focusing system employs advanced beamforming techniques to focus the ultrasound energy at the target therapy area. Beamforming involves adjusting the phase and amplitude of the signals emitted by each transducer unit in the array, effectively steering the ultrasound waves to form a focused beam. By dynamically controlling the beamforming parameters, the system can adapt the ultrasound focus to the user's unique anatomy and tissue properties.

Further, the dynamic focusing system incorporates feedback mechanisms, such as temperature sensors, motion sensors, or pressure sensors, to monitor the effectiveness of the therapy in real time. The feedback data could be used to adjust the ultrasound focusing parameters, ensuring optimal therapy delivery while minimizing the risk of adverse effects.

The dynamic focusing system would rely on sophisticated control algorithms that process real-time or near real-time imaging and feedback data to adjust the beamforming parameters and transducer positions. These algorithms could utilize machine learning techniques to optimize therapy delivery based on individual patient characteristics and treatment history.

In summary, a dynamic ultrasound focusing system combines an adaptive array of transducers, real-time or near real-time tissue imaging, advanced beamforming techniques, feedback mechanisms, and intelligent control algorithms to adaptively focus ultrasound energy on the target therapy area while considering the user's unique anatomy and tissue properties. This ensures accurate and efficient therapy delivery, potentially improving treatment outcomes and minimizing adverse effects.

An embodiment of the present device leverages advanced beamforming techniques as part of the dynamic ultrasound focusing system. Specifically, beamforming involves adjusting the phase and amplitude of the signals emitted by each transducer unit in the array. This adjustment is orchestrated in such a way that the ultrasound waves emitted from the transducers can be steered to intersect and intensify at a specific focal point, forming a concentrated beam of ultrasound energy on the target therapy area.

For instance, consider a target therapy area within the liver. To target this region, each transducer unit in the array first produces a timed signal. However, the phase and amplitude of these signals are not identical; rather, they are intentionally varied based on the location of the transducer unit and the target region within the liver. The signals from the transducers located closer to the liver might be delayed compared to those located further away, ensuring that all the signals converge at the target region simultaneously. The amplitude of the signals can be similarly adjusted, allowing for the creation of a focused beam at the target therapy area.

Real-time or near real-time imaging and feedback from sensors are utilized to dynamically control the beamforming parameters. As therapy proceeds, the device collects feedback data about the therapy's effectiveness and the patient's anatomical changes. This data is then used to adapt the ultrasound focus to the patient's unique anatomy and tissue properties.

The control algorithms that adjust the beamforming parameters are based on this real-time or near real-time data. These algorithms may include machine learning techniques to further optimize the beamforming process, allowing for adjustments to be made based on individual patient characteristics and treatment history.

For instance, if temperature sensors detect an unexpected rise in the local tissue temperature, the control algorithm could react by reducing the amplitude of the signals from the transducer units, thereby lowering the intensity of the ultrasound beam.

By implementing such an advanced beamforming technique, the dynamic ultrasound focusing system can deliver highly targeted and efficient therapy, potentially improving treatment outcomes while minimizing the occurrence of adverse effects.

In an embodiment, the dynamic focusing system for the ultrasound therapy device is designed to enhance the effectiveness and versatility of the device by allowing the transducer units to adjust their positions and orientations relative to each other, ensuring the optimal contact with the user's body surface during therapy.

Accordingly, the ultrasound therapy device contains an array of ultrasound transducer units, which are responsible for generating and transmitting ultrasound waves to the target therapy area. Each transducer unit in the array can operate independently or in coordination with other units, allowing them to focus on specific areas or distribute the ultrasound energy evenly across the treatment area.

The ultrasound transducer units are mounted on a substrate that is designed to be flexible, stretchable, or otherwise adjustable. This allows the substrate and the transducer units to conform to the contours and shape of the user's body surface, ensuring that the ultrasound energy is directed accurately and efficiently into the target therapy area.

The adaptive array of transducers is designed to allow each transducer unit to adjust its position and orientation relative to other units in the array. This can be achieved through mechanical, electromechanical, or other suitable actuation systems that move and orient the transducer units based on the user's body shape, therapy requirements, and real-time or near real-time feedback from sensors.

In an embodiment, the adaptive array of transducers could use real-time or near real-time feedback from sensors, such as ultrasound imaging sensors, to monitor the effectiveness of the therapy and adjust the transducer units' positions and orientations accordingly. This ensures that the ultrasound energy is focused on the target therapy area with maximum efficiency and minimal energy loss.

Further, the adaptive array of transducers can be customized based on the user's specific therapy needs, body shape, and preferences. Users or healthcare professionals can adjust the array configuration, transducer unit positions, and orientations to achieve the desired therapy outcomes.

By conforming to the user's body surface and adjusting the transducer units' positions and orientations, the adaptive array of transducers ensures optimal contact and energy transmission during therapy. This can lead to more effective treatments, reduced discomfort, and faster therapy progress.

By implementing an Adaptive Array of Transducers in the ultrasound therapy device, the system can deliver more accurate and efficient therapy to users, improving treatment outcomes and user experience. This dynamic focusing system can adapt to different body shapes, therapy needs, and preferences, making it a versatile and valuable addition to the device.

The present disclosure further describes examples of array configurations:

1. Linear Array: In this configuration, transducer units are aligned in a straight line. A linear array can provide focused and targeted ultrasound therapy for specific regions, such as a muscle or tendon, by concentrating the ultrasound energy along the length of the array.

2. Curved Array: A curved array features transducer units arranged along a curved path, which helps to conform to the contours of the body and distribute ultrasound energy over a broader area. This configuration can be particularly suitable for treating larger, curved body surfaces, such as the lower back or the shoulder.

3. Annular Array: Transducer units in an annular array are arranged in concentric circles or rings. This configuration can create a focused beam of ultrasound energy at various depths, allowing for more precise control over the therapy's penetration and targeting of deep tissue structures.

4. Phased Array: In a phased array configuration, transducer units are typically arranged in a rectangular or square grid, with each unit emitting ultrasound waves at slightly different times (phases). By controlling the phase differences, the array can steer and focus the ultrasound energy in a specific direction, providing precise targeting and control over the therapy area.

5. Random Array: A random array features transducer units arranged in an irregular or non-uniform pattern, providing a more diffuse distribution of ultrasound energy across the target area. This configuration can be useful for treating larger regions or for applications where a more uniform energy distribution is desired.

6. Sparse Array: In a sparse array configuration, transducer units are spaced apart, with gaps between the units. This arrangement can reduce the overall power requirements of the system, while still delivering effective therapy by focusing ultrasound energy at specific points or along specific lines within the target area.

These array configurations can be combined or adapted based on the specific therapy requirements, body shapes, and user preferences. The key is to design an array configuration that allows for optimal energy transmission, effective targeting, and efficient therapy delivery for various therapy needs and body surfaces.

Adaptive array configuration: An adaptive array configuration can be implemented in an ultrasound therapy device by designing a system that allows for dynamic adjustments of the transducer units' positions, orientations, and other parameters based on user-specific therapy needs, body shapes, and real-time or near real-time feedback.

Following is a detailed description of how an adaptive array configuration can be implemented:

1. Flexible, Stretchable, or Adjustable Substrate: Mount the ultrasound transducer units on a substrate that is designed to be flexible, stretchable, or otherwise adjustable. This will allow the substrate and the transducer units to conform to the contours and shape of the user's body surface, ensuring efficient and accurate ultrasound energy delivery.

2. Actuation System: Incorporate an actuation system that enables the transducer units to adjust their positions and orientations relative to each other. This can be achieved using mechanical, electro-mechanical, or other suitable actuation mechanisms. The actuation system can be controlled by a processor that receives input from sensors or user inputs to determine the necessary adjustments.

3. Real-time or near real-time Feedback: Utilize real-time or near real-time from sensors, such as ultrasound imaging sensors, pressure sensors, or other suitable sensors, to monitor the effectiveness of the therapy and the positioning of the transducer units. This information can be used to dynamically adjust the positions, orientations, or other parameters of the transducer units during the therapy session.

4. Control Algorithm: Develop a control algorithm that processes the real-time or near real-time feedback data, user inputs, and other relevant information to determine the optimal positioning, orientation, and other parameters for the transducer units. The control algorithm can consider factors such as the target therapy area, the user's body shape, and therapy goals when making adjustments.

5. User Interface: Design a user interface that allows users or healthcare professionals to customize the adaptive array configuration based on their specific therapy needs, preferences, and body shapes. The interface can provide options for adjusting the positioning, orientation, and other parameters of the transducer units, as well as visual feedback on the current configuration and therapy progress.

6. Calibration and Learning: Implement a calibration and learning system that enables the adaptive array configuration to improve over time based on user feedback, therapy outcomes, and other data. The system can learn from previous therapy sessions and adapt the control algorithm to provide better therapy outcomes for each user.

By implementing an adaptive array configuration in the ultrasound therapy device, the system can dynamically adjust to different therapy needs, body shapes, and preferences, ensuring optimal energy transmission and effective therapy delivery. This adaptive approach can enhance treatment outcomes, user experience, and the overall versatility of the device.

To implement beamforming techniques in the dynamic focusing system of an ultrasound therapy device, the following steps and components should be considered:

1. Individual Control of Transducer Units: Ensure that each transducer unit in the array can be controlled independently. This will allow for precise adjustments to the phase and amplitude of the signals emitted by each unit, which is critical for effective beamforming. An individual control system can be achieved using dedicated drivers and amplifiers for each transducer unit or by employing a multiplexing technique that enables the control of multiple units with a single driver.

2. Signal Generation and Timing Control: Implement a signal generation system that can produce the required ultrasound frequencies and control the timing of the signals transmitted by each transducer unit. This system can be based on digital signal processors (DSPs), field-programmable gate arrays (FPGAs), or other suitable hardware components. The signal generation system should be capable of modulating the phase and amplitude of each transducer unit's signal independently.

3. Real-time or near real-time Feedback: Incorporate sensors that provide real-time or near real-time on the ultrasound energy's focus, penetration, and distribution in the target therapy area. These sensors can include ultrasound imaging sensors, vibration sensors, or other suitable sensors. The real-time feedback, real-time or near real-time be used to adjust the beamforming parameters during the therapy session, ensuring optimal energy delivery and therapy effectiveness.

4. Beamforming Algorithm: Develop a beamforming algorithm that processes the real-time or near real-time feedback data, user inputs, and other relevant information to determine the optimal phase and amplitude adjustments for each transducer unit. The beamforming algorithm can consider factors such as the target therapy area, the user's unique anatomy, tissue properties, and therapy goals when making adjustments. The algorithm can be implemented using software running on a DSP, FPGA, or other suitable hardware platforms.

5. User Interface and Customization: Design a user interface that allows users or healthcare professionals to customize the beamforming parameters based on their specific therapy needs, preferences, and anatomical features. The interface can provide options for adjusting the focus depth, focus width, and other parameters related to beamforming, as well as visual feedback on the current focus and therapy progress.

6. Calibration and Learning: Implement a calibration and learning system that enables the beamforming techniques to improve over time based on user feedback, therapy outcomes, and other data. The system can learn from previous therapy sessions and adapt the beamforming algorithm to provide better therapy outcomes for each user.

By incorporating beamforming techniques into the dynamic focusing system of the ultrasound therapy device, the system can adapt the ultrasound focus to the user's unique anatomy and tissue properties, ensuring more accurate and efficient therapy delivery. This can lead to improved treatment outcomes and a better user experience.

In an embodiment, the ultrasound transducer units are piezoelectric or electrostrictive elements that convert electrical energy into mechanical vibrations, generating ultrasound waves. The ultrasound transducer units should be designed to generate shockwave therapy frequencies and intensities appropriate for the target therapy areas.

In an embodiment, the device includes an adaptive substrate that is a flexible, stretchable, or adjustable substrate to mount the ultrasound transducer units, allowing them to conform to the user's body surface and maintain optimal contact during therapy. The substrate material should be biocompatible, durable, and provide good acoustic coupling.

In an embodiment, the device includes an actuation system that is mechanical, electromechanical, or other suitable actuation mechanisms that enable the ultrasound transducer units to adjust their positions and orientations relative to each other based on the adaptive array configuration.

In an embodiment, the system includes digital signal processors (DSPs), field-programmable gate arrays (FPGAs), or other suitable hardware components to generate the required ultrasound frequencies, control the timing of the signals transmitted by each transducer unit, and modulate the phase and amplitude of each unit's signal independently.

In an embodiment, the device includes real-time or near real-time feedback sensors such as ultrasound imaging sensors, vibration sensors, or other suitable sensors to provide real-time or near real-time on the ultrasound energy's focus, penetration, and distribution in the target therapy area.

In an embodiment, the device includes a power supply unit to provide the necessary electrical energy for the transducer units, signal generation and control system, actuation system, and other electronic components.

In an embodiment, the device includes a microprocessor, microcontroller, or other suitable processing units to execute the control algorithms for beamforming, adaptive array configuration, and other system functionalities.

In an embodiment, the device includes memory components to store the control algorithms, user settings, therapy data, and other relevant information.

In an embodiment, the device includes a user interface, such as a touchscreen display, buttons, or other input/output devices, to allow users or healthcare professionals to interact with the system, customize settings, and receive feedback on therapy progress.

In an embodiment, the device includes a communication module, such as Wi-Fi, Bluetooth, or other wireless communication protocols, to enable remote monitoring and control, data transfer, and integration with other devices or telemedicine platforms.

In an embodiment, the device includes a wearable structure or harness to secure the device to the user's body during therapy sessions. The wearable structure should be adjustable, and comfortable, and provide proper support for the transducer array and associated hardware components.

In an embodiment, the device may include a haptic feedback system. If implementing haptic feedback for self-administration guidance, a system of actuators, such as vibration motors, generates tactile sensations and guides the user during therapy sessions.

In an embodiment, the device may include an Augmented Reality (AR) system. If implementing AR guidance, a compatible smartphone or wearable AR device to provide real-time or near real-time visual instructions and information to users during therapy sessions.

In some aspects, the system comprises a photoacoustic imaging module and an ultrasound therapy module. The photoacoustic imaging module utilizes laser-induced acoustic waves to generate high-resolution images of biological tissues. In operation, a laser beam is directed onto the tissue, which absorbs the laser energy and undergoes thermal expansion. This rapid expansion generates ultrasound waves that can be detected using an ultrasound transducer. The detected ultrasound signals are processed to reconstruct images of the tissue, providing detailed anatomical and functional information.

The photoacoustic images produced by the imaging module offer several advantages. First, they provide high-resolution visualization of the tissue, allowing for accurate identification and localization of target tissues or pathologies. The imaging module can differentiate between different tissue types based on their optical absorption properties, revealing structural details and potential abnormalities. Additionally, the photoacoustic imaging can assess functional parameters such as blood perfusion and oxygen saturation, providing valuable insights into tissue metabolism and vascular dynamics.

The photoacoustic imaging module includes a laser source that emits laser pulses at specific wavelengths optimized for tissue penetration and contrast. The laser pulses can be delivered to the tissue through different methods, such as optical fibers or light-emitting diodes (LEDs). The imaging module also includes an ultrasound transducer array or a single transducer that detects the generated ultrasound signals. These signals are then amplified, filtered, and processed using signal processing techniques to reconstruct the photoacoustic images.

During a treatment session, the photoacoustic imaging module captures real-time or near real-time images of the target tissue or pathology. These images are continuously monitored and analyzed to assess treatment response and guide the ultrasound therapy. For example, the images may reveal the extent of tissue damage, the distribution of therapeutic agents, or the change in vascularization. This real-time or near real-time allows for dynamic adjustments of the ultrasound therapy parameters, optimizing the treatment delivery and minimizing adverse effects.

Some transducer unit technologies including those described below can be used to enable the shockwave therapy.

1. Piezoelectric Transducers: These transducers use piezoelectric materials, such as lead zirconate titanate (PZT) or single-crystal relaxor ferroelectrics, that generate mechanical vibrations when subjected to an electric field. They are commonly used in ultrasound devices due to their high sensitivity, wide frequency range, and ease of fabrication.

2. Capacitive Micromachined Ultrasonic Transducers (CMUTs): CMUTs are micro-electro-mechanical systems (MEMS) devices that use electrostatic forces between a thin membrane and a fixed electrode to generate ultrasound waves. CMUTs offer advantages such as broad bandwidth, high sensitivity, and the potential for integration with electronic circuits on a single chip.

3. Piezoelectric Micromachined Ultrasonic Transducers (PMUTs): PMUTs are MEMS devices that utilize piezoelectric thin films to generate ultrasound waves. They offer similar advantages to CMUTs, with the added benefit of using piezoelectric materials, resulting in lower voltage requirements and improved power efficiency.

4. Electrostrictive Transducers: These transducers use electrostrictive materials, such as polyvinylidene fluoride (PVDF) or its copolymers, which change their shape in response to an electric field. Electrostrictive transducers can offer high sensitivity and low acoustic impedance, making them suitable for certain ultrasound applications.

5. Magnetostrictive Transducers: Magnetostrictive transducers utilize materials that change their shape in response to a magnetic field, such as Terfenol-D or Metglas. While not as common in ultrasound applications, they can offer high power output and are suitable for generating lower-frequency ultrasound waves.

6. Air-coupled Transducers: Air-coupled transducers use matching layers and materials designed for efficient transmission of ultrasound energy through air or other gases, rather than through a coupling medium like gel or water. These transducers can be useful in applications where direct contact with the user's body is not desired or practical.

7. Phased Array Transducers: Phased array transducers consist of multiple small transducer elements arranged in a specific pattern. By controlling the timing and phase of the signals emitted by each element, the ultrasound waves can be steered and focused electronically, without the need for mechanical adjustments. Phased array transducers can be created using piezoelectric, CMUT, or other suitable transducer technologies.

These transducer unit technologies can be used individually or in combination to create a versatile and effective shockwave therapy device. The choice of transducer technology will depend on factors such as the desired frequency range, power output, size constraints, and integration with other components.

In an embodiment, an AI-driven personalized therapy system leverages artificial intelligence (AI) to analyze a user's unique physiological characteristics, medical history, and response to previous therapy sessions to create a personalized therapy plan. The AI-driven therapy continually adapts and optimizes treatment parameters based on real-time or near real-time feedback, significantly improving the overall effectiveness and safety of the therapy.

The present disclosure further describes the implementation and operational details of the AI-driven personalized therapy system.

1. Data Collection: The first step in implementing AI-driven personalized therapy is to collect relevant data about the user. This may include demographic information, medical history, physiological parameters (e.g., tissue density, blood flow), and previous therapy session data (e.g., treatment settings, user feedback, treatment outcomes). Additional data may be gathered through wearable sensors, imaging techniques, or user input during therapy sessions.

2. Data Preprocessing: Preprocess the collected data to ensure it is clean, accurate, and ready for analysis. This may involve dealing with missing values, removing outliers, and normalizing data to ensure consistency.

3. Feature Extraction: Identify relevant features within the collected data that can be used to predict treatment outcomes and personalize therapy settings. This may include patterns in physiological parameters, correlations between therapy settings and outcomes, and trends in user feedback.

4. Machine Learning Model: Develop a machine learning model that can analyze the collected data and extracted features to create personalized therapy plans. This model may utilize supervised or unsupervised learning techniques, such as regression, classification, clustering, or deep learning, depending on the nature of the data and the desired outcomes.

5. Model Training: Train the machine learning model using historical data from a large sample of users with varying physiological characteristics and treatment needs. The model should be able to identify patterns and relationships in the data that can be used to optimize therapy settings for individual users.

6. Real-time or near real-time Feedback Integration: During therapy sessions, continuously collect real-time or near real-time data (e.g., user comfort, tissue temperature, treatment progress) and use this information to update the machine learning model. This allows the AI-driven therapy system to adapt and optimize treatment settings based on the user's response to therapy, ensuring optimal outcomes and safety.

7. User Interface: Design a user interface for the therapy device that allows users to interact with the AI-driven personalized therapy system. This interface may include options for users to input their medical history, receive personalized therapy recommendations, monitor their progress during therapy sessions, and provide feedback on treatment outcomes.

8. Continuous Improvement: Continuously update and improve the machine learning model based on new data and user feedback. This ensures the AI-driven personalized therapy system remains accurate and effective over time, adapting to new research findings, treatment techniques, and user needs.

By implementing the aforementioned steps, the AI-driven personalized therapy system can create customized treatment plans for each user, optimizing therapy settings based on their unique needs and preferences. This adaptive approach has the potential to improve treatment outcomes, increase user satisfaction, and minimize the risk of adverse effects, making the therapy more effective and safe.

Some embodiments provide an ultrasound-triggered drug delivery system that combines ultrasound therapy with targeted drug delivery. In an embodiment, this ultrasound-triggered drug delivery system uses microcapsules or nanoparticles containing medication that can be selectively released at the target therapy area upon exposure to ultrasound waves. Further, the present disclosure describes the implementation details of the ultrasound-triggered drug delivery system.

1. Drug Encapsulation: Encapsulate the desired therapeutic agent within microcapsules or nanoparticles. These carriers can be made from a variety of materials, such as liposomes, polymers, or inorganic materials, depending on the drug properties, release requirements, and biocompatibility. The encapsulation process ensures that the drug remains protected until triggered by the ultrasound.

2. Drug Administration: Administer the encapsulated drug to the patient through an appropriate method, such as intravenous injection, local injection, or oral ingestion. The choice of administration method will depend on the drug properties, therapy area, and desired release kinetics.

3. Targeting Strategy: Implement a targeting strategy to ensure that the encapsulated drug accumulates at the target therapy area. This can be achieved through passive targeting (e.g., exploiting the enhanced permeability and retention effect in tumors) or active targeting (e.g., attaching targeting ligands to the carrier surface that bind specifically to receptors on target cells).

4. Ultrasound Therapy Device: Equip the ultrasound therapy device with appropriate settings and transducers to generate ultrasound waves that can trigger drug release from the carriers. This may involve using specific frequencies, intensities, or pulse patterns that cause mechanical or thermal effects on the carriers, leading to drug release.

5. Real-time or near real-time Imaging: Integrate real-time or near real-time techniques, such as ultrasound imaging or other imaging modalities, to monitor the drug delivery process. This provides valuable information on drug distribution, carrier accumulation at the target site, and the effectiveness of the therapy, allowing for adjustments in ultrasound parameters or treatment planning.

6. Ultrasound-triggered Drug Release: During the therapy session, direct the ultrasound waves towards the target therapy area, where the encapsulated drug has accumulated. The ultrasound waves interact with the carriers, causing them to release the drug in a controlled and localized manner. The drug release mechanism may involve mechanical disruption (e.g., cavitation, microstreaming), thermal effects (e.g., local heating, phase transition), or other mechanisms, depending on the carrier material and ultrasound settings.

7. Monitoring and Control: Monitor the therapy process, drug release, and patient response in real time. This information can be used to adjust the ultrasound parameters, drug dosage, or treatment plan as needed, ensuring optimal treatment outcomes and minimizing potential side effects.

By implementing an ultrasound-triggered drug delivery system, it is possible to achieve controlled and localized drug release at the target therapy area, potentially enhancing the therapeutic outcomes while reducing systemic side effects. This innovative approach combines the benefits of ultrasound therapy with targeted drug delivery, opening up new possibilities for treating various medical conditions.

The present disclosure further describes the integration of multi-modal therapy into the device. Multi-modal therapy integration involves combining the ultrasound therapy device with other therapeutic modalities to create a synergistic effect, potentially increasing the overall effectiveness of the treatment. These additional therapies can be delivered either simultaneously or sequentially, depending on the user's needs and therapy plan. Following is a detailed description of how such a system is implemented:

1. Therapy Modalities Selection: Choose the additional therapeutic modalities to be integrated with the ultrasound therapy device based on the target therapy area, desired outcomes, and user preferences. Some examples include electrical stimulation (e.g., Transcutaneous Electrical Nerve Stimulation, TENS), light therapy (e.g., photobiomodulation), and vibration therapy (e.g., mechanotherapy).

2. Hardware Integration: Modify the design of the ultrasound therapy device to incorporate the necessary hardware for delivering the additional therapies. This may involve integrating electrodes for electrical stimulation, light-emitting diodes (LEDs) or lasers for light therapy, and mechanical actuators for vibration therapy.

3. Control System: Develop a control system that can manage the delivery of each therapy modality, either simultaneously or sequentially. This may involve creating algorithms that coordinate the activation and intensity of each therapy component based on user input, real-time or near real-time feedback, or predefined therapy plans.

4. Real-time or near real-time Feedback Mechanisms: Incorporate feedback mechanisms, such as sensors or imaging techniques, to monitor the effectiveness of each therapy modality during treatment. This information can be used to adjust the therapy settings and optimize the combined effects of the various modalities.

5. User Interface: Design a user interface for the multi-modal therapy device that allows users to select and customize the desired therapy modalities, set treatment parameters, and monitor the progress of their therapy sessions. This interface may include visual displays, buttons, or touchscreens for easy interaction.

6. Therapy Protocols: Develop therapy protocols for the multi-modal therapy system that specify the optimal combination of therapy modalities, treatment parameters, and session durations for various conditions or user needs. These protocols can be based on clinical research, expert recommendations, or user feedback.

7. Clinical Trials and Validation: Conduct clinical trials and validation studies to evaluate the safety and efficacy of the multi-modal therapy system compared to traditional single-modality treatments. This information can be used to refine the therapy protocols, hardware design, and control algorithms, ensuring the best possible outcomes for users.

8. Continuous Improvement: Continuously update and improve the multi-modal therapy system based on new research findings, technological advancements, and user feedback. This ensures that the system remains cutting-edge and effective over time, adapting to new treatment techniques and user needs.

By implementing multi-modal therapy integration, the ultrasound therapy device can deliver a range of therapeutic modalities that work together to enhance treatment outcomes, cater to individual user needs, and potentially improve the overall effectiveness and safety of the therapy.

Some embodiments further implement the therapy modalities selection process. The therapy modalities selection process involves choosing additional therapeutic modalities to be integrated with the ultrasound therapy device. The therapy modalities selection process is based on the target therapy area, desired outcomes, and user preferences. This step is crucial in creating a multi-modal therapy system that provides optimal treatment benefits.

Examples of therapy modalities that could be considered for integration with ultrasound therapy include:

Electrical Stimulation: TENS (Transcutaneous Electrical Nerve Stimulation) or NMES (Neuromuscular Electrical Stimulation) can be used for pain relief, muscle strengthening, or improving blood circulation. These modalities involve applying electrical currents to the skin to stimulate nerves or muscles.

Light Therapy: Photobiomodulation or low-level laser therapy (LLLT) can help promote tissue repair, reduce inflammation, and alleviate pain. Light therapy involves exposing the target area to specific wavelengths of light, typically in the red or near-infrared spectrum.

Vibration Therapy: Mechanotherapy or whole-body vibration can be used to improve muscle strength, flexibility, and circulation. Vibration therapy involves applying mechanical vibrations to the body, either locally or systemically.

Thermotherapy: Applying heat (e.g., infrared therapy) or cold (e.g., cryotherapy) can help reduce pain, inflammation, and muscle spasms. Thermotherapy can be integrated into the device or used as a complementary treatment.

By carefully selecting the appropriate therapy modalities based on the factors mentioned above, a multi-modal therapy system can be created that is tailored to the specific needs of users and provides optimal treatment benefits.

Some embodiments further provide various examples of therapy modalities that could potentially be integrated with an ultrasound therapy device or used alongside it.

1. Pulsed Electromagnetic Field (PEMF) Therapy: PEMF therapy uses low-frequency electromagnetic fields to stimulate cellular functions, promote healing, reduce inflammation, and alleviate pain. This modality has been used to treat conditions like arthritis, fractures, and soft tissue injuries.

2. Extracorporeal Shockwave Therapy (ESWT): ESWT uses high-energy acoustic waves to promote tissue regeneration, reduce inflammation, and break up scar tissue. This modality has been used for treating conditions like plantar fasciitis, tendonitis, and calcific tendinitis.

3. High-Intensity Focused Ultrasound (HIFU): HIFU involves the use of focused ultrasound waves to generate localized heat, which can be used for tissue ablation or targeted drug delivery. This modality has applications in cancer treatment, tissue remodeling, and lipolysis.

4. Radiofrequency (RF) Therapy: RF therapy uses high-frequency electrical currents to generate heat in the target tissues, promoting collagen production, and tissue tightening. This modality is often used for skin tightening, wrinkle reduction, and body contouring.

5. Magnetic Resonance Therapy (MRT): MRT uses low-intensity magnetic fields to stimulate cellular functions, promote relaxation, and reduce pain. This therapy has been used to treat conditions like fibromyalgia, stress, and insomnia.

6. Hyperbaric Oxygen Therapy (HBOT): HBOT involves breathing pure oxygen in a pressurized chamber, which increases oxygen levels in the blood and promotes tissue healing, reduces inflammation, and combats infection. This modality has been used to treat conditions like diabetic foot ulcers, decompression sickness, and radiation injuries.

7. Biofeedback: Biofeedback involves monitoring physiological signals, such as heart rate, skin temperature, or muscle tension, and providing real-time or near real-time feedback to users, enabling them to learn how to control these functions voluntarily. Biofeedback can be used to manage stress, chronic pain, and various neurological disorders.

8. Acupuncture: Acupuncture involves the insertion of thin needles at specific points on the body to stimulate the nervous system, release endorphins, and promote healing. This modality has been used for pain relief, stress reduction, and treating various conditions, such as migraines, allergies, and digestive disorders.

9. Microcurrent Therapy: Microcurrent therapy uses low-level electrical currents to stimulate cellular functions, promote tissue repair, and reduce inflammation. This modality has been used to treat conditions like soft tissue injuries, neuropathy, and postoperative pain.

These additional therapy modalities can be considered for integration with an ultrasound therapy device or used in combination with ultrasound therapy to create a comprehensive multi-modal treatment plan. The selection of therapy modalities should be based on the target therapy area, desired outcomes, user preferences, and practical considerations.

Some embodiments further describe several extracorporeal energy-based treatment modalities. Several of the modalities mentioned may aid in angiogenesis, the process of forming new blood vessels, which may enable tissue healing and tissue regeneration. Electrical Stimulation (TENS/NMES) for example may promote angiogenesis through electrical stimulation which may enhance the release of vascular endothelial growth factor (VEGF), a key protein that stimulates the formation of blood vessels. Light Therapy (Photobiomodulation/LLLT), in some embodiments with near-infrared light, can induce angiogenesis by triggering the release of growth factors and enhancing cellular proliferation and migration. Pulsed Electromagnetic Field (PEMF) therapy can stimulate angiogenesis through up-regulating the expression of growth factors such as VEGF and fibroblast growth factor (FGF). Hyperbaric Oxygen Therapy (HBOT) can enhance the oxygenation of tissues, a key stimulus for angiogenesis. Hyperbaric oxygen minimises cell damage and death by reducing fluid accumulation in injured heart cells. Hyperbaric oxygen's primary effect in heart attack may be to reduce edema (fluid accumulation) in heart cells. Microcurrent therapy uses electrical currents to stimulate tissues and can enhance the release of growth factors and promote angiogenesis. Extracorporeal Magnetic Innervation (ExMI) uses magnetic fields to generate electrical currents within the body, which can induce angiogenesis. Transcranial Magnetic Stimulation (TMS) uses magnetic fields to stimulate nerve cells in the brain which can induce angiogenesis. Cold Laser Therapy or Low-Level Laser Therapy (LLLT) uses low levels of light to stimulate healing and can induce angiogenesis. Gamma Knife Radiosurgery uses focused gamma rays to treat small to medium-sized lesions and can induce angiogenesis. Interferential Current Therapy (IFC) uses a mid-frequency electrical signal and a current produced by the interference of two high-frequency signals, and its amplitude variations can effectively stimulate large sensory nerves and can induce angiogenesis. Pulsed Dye Laser Therapy destroys specific blood vessels without significantly damaging surrounding tissue and can induce angiogenesis. Cavitational Ultrasonolysis (histotripsy) uses pulsed ultrasound to produce controlled mechanical disruption of targeted tissues and can induce angiogenesis.

Examples of adjustable treatment parameters for some of these treatment modalities that might help to promote angiogenesis include but are not limited to:

Pulsed Electromagnetic Field Therapy (PEMF): In a device for PEMF therapy to promote angiogenesis, the electromagnetic field generator may be configured to generate pulses with an adjustable frequency range of 10 Hz to 50 Hz. The intensity could be varied from 1 Gauss to 20 Gauss and the therapy duration may be between 15 and 60 minutes.

For example, a PEMF therapy device may include a processor configured to control the electromagnetic field generator to generate pulses at a frequency of 25 Hz, an intensity of 10 Gauss, for a duration of 30 minutes per therapy session, which may stimulate angiogenesis in the targeted area.

Transcutaneous Electrical Nerve Stimulation (TENS)/Neuromuscular Electrical Stimulation (NMES): For a device delivering TENS/NMES to promote angiogenesis, parameters could include a pulse width between 100 and 400 microseconds, frequency between 2-50 Hz, and intensity that provides a tolerable sensation without discomfort. The therapy could last from 20 to 60 minutes.

An embodiment of a TENS/NMES device for angiogenesis may comprise an electrical stimulator that generates electrical pulses with a pulse width of 200 microseconds, a frequency of 30 Hz, and at an intensity that is comfortable for the user, for a duration of 45 minutes per therapy session.

Light Therapy (Photobiomodulation/LLLT): In a LLLT device for angiogenesis, the light source might emit light within the red to near-infrared wavelength range (600-1000 nm), power density of 0.1-0.5 W/cm², energy density of 5-15 J/cm², and duration of 1-3 minutes per point.

For example, a photobiomodulation device may utilize a light source configured to emit light with a wavelength of 660 nm, a power density of 0.3 W/cm², at an energy density of 10 J/cm², for a duration of 2 minutes per point, which can stimulate angiogenesis in the targeted area.

Extracorporeal Magnetic Innervation (ExMI): For ExMI, the device could produce a magnetic field of 1-2 Tesla, with a pulse frequency of 10-30 Hz, for a duration of 15-30 minutes.

An ExMI device may generate a magnetic field of 1.5 Tesla, at a pulse frequency of 20 Hz, for a duration of 20 minutes per therapy session, which can stimulate angiogenesis in the treatment area.

Transcranial Magnetic Stimulation (TMS): In a TMS device, pulses might be delivered at an intensity of 80-110% of the motor threshold, at a frequency of 5-10 Hz for repetitive TMS, with 500-2000 pulses per session, for a session duration of 15-30 minutes.

A TMS device may deliver pulses at an intensity of 90% of the motor threshold, at a frequency of 10 Hz for repetitive TMS, with 1000 pulses per session, for a session duration of 20 minutes, which can help to stimulate angiogenesis in the brain.

Cold Laser Therapy or Low-Level Laser Therapy (LLLT): For LLLT, the device could deliver light within a wavelength of 600-900 nm, with a power density of 0.1-1 W/cm², an energy density of 4-12 J/cm², for a duration of 30 seconds-2 minutes per point.

For example, a cold laser therapy device may utilize a laser source emitting light with a wavelength of 800 nm, with a power density of 0.5 W/cm², at an energy density of 8 J/cm², for a duration of 1 minute per point of treatment, which can promote angiogenesis in the treatment area.

Some embodiments pertain to an apparatus specifically designed to deliver extracorporeal energy therapy, which is aimed at inducing angiogenesis in the tissue of a patient.

The apparatus comprises a casing oriented towards the skin of the patient. This casing serves as a structural framework and facilitates the application of the therapy directly to the patient's skin.

Integral to the apparatus is an assembly of transducer units, strategically placed on the skin-facing side of the casing. These transducer units are capable of generating and delivering extracorporeal energy therapy to a specified treatment region on the patient. The therapeutic modalities which can be utilized in the extracorporeal energy-based therapy may include, but are not limited to, Pulsed Electromagnetic Field Therapy, Electrical Stimulation (TENS/NMES), Light Therapy (Photobiomodulation/LLLT), Extracorporeal Magnetic Innervation (ExMI), Transcranial Magnetic Stimulation, and Cold Laser Therapy or Low-Level Laser Therapy.

The apparatus also includes a communication system. This system ensures that the necessary information exchange between the apparatus and the external devices, or between different components of the apparatus, is achieved efficiently.

An energy source is incorporated into the apparatus to provide the necessary power for the functioning of the transducer units and other electronic components within the apparatus.

In the heart of the apparatus lies a processor. The processor is designed to execute a series of instructions which govern the operational dynamics of the apparatus.

The first instruction involves evaluating the patient's non-urgent health condition. This is achieved by utilizing data received from a physiological sensor. This sensor could be either connected to the patient or integrated within the apparatus itself.

Next, the processor gauges the proximity of each transducer unit in the assembly to the patient's skin. This helps to ensure optimal positioning of the transducer units for delivering the therapy.

A critical instruction executed by the processor involves verifying that the transducer units have sufficient contact with the patient's skin. This is essential for ensuring effective delivery of the therapy.

Once the positioning and contact verification are done, the processor conveys therapy-related data pertaining to extracorporeal wave energy to the transducer units. This data guides the transducer units in the generation and delivery of the therapy.

Lastly, the processor initiates the production of extracorporeal energy therapy, targeted at the treatment region on the patient. The therapy produced is optimized to promote angiogenesis within the patient's tissue. The execution of this final instruction signifies the operational culmination of the apparatus.

In summary, these embodiments provide a comprehensive solution for administering extracorporeal energy therapy targeted at inducing angiogenesis in a patient's tissue. The unique combination of components and the smart instructions executed by the processor ensure that the therapy is delivered optimally for the maximum therapeutic benefit.

Examples of an apparatus for administering extracorporeal energy therapy targeted at inducing angiogenesis in a patient's tissue, may include the following components:
A casing with a side oriented towards the patient's skin;
An assembly of transducer units mounted on the skin-facing side, wherein said assembly is devised to produce and deliver extracorporeal energy therapy to a specified treatment region on a patient. The extracorporeal energy-based therapy may incorporate one or more therapeutic modalities including Pulsed Electromagnetic Field Therapy, Electrical Stimulation (TENS/NMES), Light Therapy (Photobiomodulation/LLLT), Extracorporeal Magnetic Innervation (ExMI), Transcranial Magnetic Stimulation, and Cold Laser Therapy or Low-Level Laser Therapy;
A system for communication;
A source of energy;
A processor designed to execute the following sequences of instructions:
Evaluating the patient's non-urgent health condition utilizing data received from a physiological sensor that is either connected to the patient or integrated within the apparatus;
Gauging the proximity of each transducer unit in the assembly to the patient's skin;
Verifying that the transducer units have sufficient contact with the patient's skin for therapy;
Conveying therapy-related data pertaining to extracorporeal wave energy to the transducer units;
Initiating the production of extracorporeal energy therapy targeted at the treatment region on the patient, with the therapy optimized to promote angiogenesis within the patient's tissue.

Some embodiments further describe the implementation details of haptic feedback for self-administration guidance.

The haptic feedback for self-administration guidance is a feature that aims to help users correctly position and use the ultrasound therapy device during self-administration. This system uses tactile feedback, such as vibrations, to provide real-time or near real-time guidance on device positioning and adjustments. The present disclosure further provides a detailed description of how such a system could be implemented:

1. Sensor Integration: Equip the ultrasound therapy device with sensors that can detect the relative positioning of the device with respect to the target therapy area. These sensors could include ultrasonic sensors, infrared sensors, or other suitable technologies to determine the distance, orientation, and alignment of the device concerning the therapy area.

2. Haptic Feedback Hardware: Integrate haptic feedback components, such as vibration motors or piezoelectric actuators, into the wearable structure of the ultrasound therapy device. These components should be strategically placed to provide clear and distinguishable tactile feedback to the user.

3. Real-time or near real-time Position Monitoring: Develop a real-time or near real-time monitoring system that continuously tracks the position and orientation of the ultrasound therapy device using integrated sensors. This system should analyze the sensor data to determine whether the device is correctly positioned over the target therapy area and aligned for optimal therapy delivery.

4. Feedback Algorithms: Develop algorithms that translate the real-time or near real-time position information into appropriate haptic feedback signals. These algorithms should determine when and how to activate the haptic feedback components to guide the user in adjusting the device's position or orientation.

5. User Interface: Design a user interface for the ultrasound therapy device that visually displays the device's position and alignment status, complementing the haptic feedback system. This interface could provide visual cues or instructions on how to adjust the device for optimal therapy delivery.

6. Training and Calibration: Develop a training and calibration protocol for users to familiarize themselves with the haptic feedback system and understand the different tactile signals. This protocol may include guided practice sessions, interactive tutorials, or step-by-step instructions.

7. Customization Options: Allow users to customize the haptic feedback settings, such as vibration intensity or frequency, to suit their personal preferences and comfort levels. This can help ensure that users find the haptic feedback system useful and user-friendly.

8. Continuous Improvement: Regularly update and refine the haptic feedback system based on user feedback, technological advancements, and new research findings. This ensures that the system remains effective and user-friendly over time, adapting to evolving user needs and expectations.

By implementing haptic feedback for a self-administration guidance system, users can receive real-time or near real-time guidance on the optimal positioning and use of the ultrasound therapy device, ensuring accurate and effective therapy delivery. This feature can help enhance treatment outcomes, increase user confidence, and improve overall user satisfaction with the device.

Some embodiments include an Augmented Reality (AR) guidance system to provide real-time or near real-time visual instructions to users on the correct positioning and use of the ultrasound therapy device. It can also display progress, therapy settings, and safety information during the treatment.

Further, some embodiments include the implementation details of an Augmented Reality (AR) guidance system.

1. AR hardware: Determine the preferred AR hardware for the guidance system. This could involve using a smartphone with an AR-compatible camera or wearable AR devices like smart glasses or headsets. The chosen hardware should be easily accessible, user-friendly, and compatible with the ultrasound therapy device.

2. AR Software: Develop an AR application or software module that can process data from the ultrasound therapy device and the chosen AR hardware. This software should be capable of generating real-time or near real-time visual overlays that provide clear guidance on device positioning, usage, and therapy information.

3. Sensor Integration: Equip the ultrasound therapy device with sensors that can track its position and orientation relative to the target therapy area. These sensors could include accelerometers, gyroscopes, or magnetic sensors that can communicate with the AR software to provide accurate positioning data.

4. Device Registration: Develop a registration process that allows the AR software to recognize and track the ultrasound therapy device in the user's field of view. This process could involve using fiducial markers, visual patterns, or machine learning algorithms to identify the device and its position.

5. Real-time or near real-time Visualization: Create visual overlays that accurately represent the ultrasound therapy device, the target therapy area, and the desired device positioning. These overlays should be updated in real-time or near real-time sensor data to provide dynamic guidance to the user.

6. User Interface: Design a user interface for the AR guidance system that displays relevant therapy information, such as progress, settings, and safety alerts. This interface should be intuitive and easy to navigate, allowing users to access and interpret the information quickly.

7. Training and Support: Develop training materials and support resources to help users understand and use the AR guidance system effectively. This could include tutorials, interactive demonstrations, or step-by-step instructions that familiarize users with the system's features and functions.

8. Customization and Personalization: Allow users to customize the AR guidance system based on their preferences and needs. This could involve adjusting the visual overlays' appearance, modifying the displayed therapy information, or choosing between different guidance modes.

9. Continuous Improvement: Regularly update and refine the AR guidance system based on user feedback, technological advancements, and new research findings. This ensures that the system remains effective and user-friendly over time, adapting to evolving user needs and expectations.

By implementing an Augmented Reality (AR) Guidance system, users can receive real-time or near real-time visual instructions and information on the correct positioning and use of the ultrasound therapy device. This feature can help improve treatment outcomes, increase user confidence, and enhance overall user satisfaction with the device.

According to an embodiment herein, the present device has telemedicine compatibility to enable remote monitoring and control of the ultrasound therapy device by healthcare professionals. This feature allows professionals to observe therapy sessions, adjust settings, and provide guidance in real-time or near real-time, enhancing safety and effectiveness while offering users the convenience of receiving assistance at home. The present disclosure further describes the implementation details of telemedicine compatibility.

1. Internet Connectivity: Equip the ultrasound therapy device with internet connectivity capabilities, such as Wi-Fi or cellular data, to facilitate communication with remote healthcare professionals. This connectivity enables the transmission of data between the device and the healthcare professional's computer or mobile device.

2. Secure Data Transmission: Implement secure data transmission protocols to ensure that the information exchanged between the ultrasound therapy device and the healthcare professional's device is protected from unauthorized access or tampering. This could involve using encryption, authentication, and secure communication channels.

3. Remote Monitoring Interface: Develop a user interface for healthcare professionals that allows them to remotely monitor the therapy session. This interface should display real-time or near real-time data from the ultrasound therapy device, such as sensor readings, device positioning, therapy progress, and any alerts or warnings. The interface should also enable healthcare professionals to access historical therapy data and user profiles.

4. Remote Control Capabilities: Allow healthcare professionals to remotely adjust the settings of the ultrasound therapy device during a therapy session. This could involve changing the intensity, frequency, or duration of the therapy, or activating/deactivating specific therapy modalities, based on the user's needs and progress.

5. Two-way Communication: Establish a two-way communication channel between the user and the healthcare professional, enabling them to discuss the therapy session, ask questions, or receive guidance in real time. This communication could be facilitated through audio, video, or text messaging within the remote monitoring interface.

6. Integration with Telemedicine Platforms: Design the telemedicine compatibility feature to be compatible with existing telemedicine platforms and electronic health record systems. This ensures seamless integration with healthcare professionals' existing workflows and tools, facilitating the adoption of remote monitoring and control capabilities.

7. User Consent and Privacy: Implement features that allow users to provide informed consent for remote monitoring and control, and manage their privacy settings. This could involve obtaining explicit consent before initiating remote monitoring, offering options to control the types of data shared, and providing clear explanations of how the data will be used.

8. Training and Support: Develop training materials and support resources to help healthcare professionals understand and use the telemedicine compatibility feature effectively. This could include tutorials, interactive demonstrations, or step-by-step instructions that familiarize professionals with the system's features and functions.

9. Continuous Improvement: Regularly update and refine the telemedicine compatibility feature based on user feedback, technological advancements, and new research findings. This ensures that the system remains effective, user-friendly, and in compliance with evolving regulations and standards.

By implementing telemedicine compatibility, the users can benefit from professional assistance during therapy sessions, while healthcare professionals can remotely monitor and control the ultrasound therapy device, enhancing treatment safety and effectiveness. This feature can also help bridge the gap between in-person and remote care, offering users greater convenience and flexibility in receiving therapy.

An embodiment provides a drug-coated balloon for medical applications. The drug-coated balloon includes a flexible balloon structure and an exterior surface. The flexible balloon structure is capable of inflation and deflation. The exterior surface of said balloon structure is coated with a therapeutic drug formulation. The drug formulation is configured to be locally delivered to a body tissue upon contact and extracorporeal stimulation around the time that the balloon is inflated. The therapeutic drug formulation includes a pharmacologically active agent selected from the group consisting of antiproliferative agents, anti-inflammatory agents, antineoplastic agents, antiplatelet agents, anticoagulant agents, fibrinolytic agents, thrombin inhibitors, growth factor inhibitors, and combinations thereof. The exterior surface further comprises a polymer matrix sensitive to ultrasound waves for controlling the release of the therapeutic drug formulation. The ultrasound-sensitive polymer matrix is configured to degrade and release the drug formulation upon receiving ultrasound stimulation. The balloon structure is adapted for use in angioplasty or stent delivery procedures.

An embodiment provides a method for delivering a therapeutic drug to body tissue. The method includes a step of inflating the drug-coated balloon of any preceding claim in proximity to the target tissue. Further, the method includes a step of applying extracorporeal ultrasound stimulation to said balloon to induce degradation of the polymer matrix and release of the therapeutic drug.

Another embodiment provides ultrasound therapy to improve drug absorption. This effect may be achieved through techniques known as sonophoresis or phonophoresis, which can help improve drug absorption. This technique uses ultrasound waves to enhance the absorption of topically applied drugs. It works based on several principles:
1. Cavitation: This is the formation, growth, and implosive collapse of bubbles in a liquid. This effect of ultrasound can temporarily disrupt the skin's outer layer, thereby increasing its permeability.
2. Thermal Effects: The application of ultrasound can lead to a rise in tissue temperature, leading to vasodilation. This can increase local blood flow and subsequently promote the delivery of drugs.
3. Acoustic Streaming: This involves the movement of fluid induced by the ultrasound wave, which can help distribute the drug more evenly and promote its penetration into tissues.
   In addition to enhancing transdermal drug delivery, ultrasound can also be used to improve drug absorption in other ways:
1. Drug Targeting: Ultrasound, particularly when combined with microbubbles (small gas-filled bubbles), can be used to selectively deliver drugs to specific areas within the body. The drug is either loaded into these microbubbles or administered simultaneously. Once the microbubbles reach the target area, an ultrasound wave is used to burst them, releasing the drug at the desired location.
2. Enhanced Permeability and Retention Effect: For certain conditions like cancer, ultrasound can enhance the permeability of the vasculature in tumor tissues, which allows nanoparticles or drugs to penetrate the tissue more effectively. This effect can be maximized with the use of microbubbles.
3. Sonoporation: This is a technique in which ultrasound is used to create temporary pores in cell membranes, enhancing the uptake of drugs into cells. This is particularly useful for gene therapy and chemotherapy, where drugs often need to be delivered directly into cells.
4. Thrombolysis: Ultrasound can be used to enhance the effectiveness of thrombolytic (clot-dissolving) drugs. The mechanical force of the ultrasound waves can help to break up the clot, aiding the drug in dissolving it more efficiently.

Some embodiments comprise a wearable structure to hold the device in place. Said wearable structure may comprise one or more material. In an embodiment the wearable structure of the device comprises a lightweight, breathable, and hypoallergenic material, designed to enhance user comfort during prolonged usage and exhibit flexibility to various body shapes and sizes. Such materials may include but is not limited to: Neoprene, spandex/lycra, polyurethane foam, silicone rubber and/or textiles with silver thread.

The wearable structure of the device is built on a modular framework which allows for the interchangeable arrangement of its components. This framework facilitates customization of the device to meet individual user requirements and body specifications.

The key components of the device, such as the ultrasound transducer units, energy source, and communication module, are designed as individual modules that can be conveniently added, removed, or repositioned within the wearable structure. Each module interfaces with the others through a universal connection mechanism, which can include physical connectors, wireless interfaces, or a combination of both.

For example, the ultrasound transducer units could be contained within individual modules, which could be repositioned or replaced within designated slots or areas on the wearable structure. This modular approach allows the user to alter the positioning of the transducer units to focus the ultrasound therapy on the targeted therapy area. Additional modules, such as supplementary transducer units or sensors, could also be added to the device as needed.

The wearable structure itself is equipped with an adjustable sizing mechanism that can cater to a wide range of body sizes and shapes. This could involve elastic components, adjustable straps, or a combination of both. The structure also incorporates a series of designated attachment points for the various modules. These attachment points could be uniformly distributed to allow for maximum customization, or strategically located to align with common therapy areas.

The wearable structure of the device is designed to contour naturally to the curves of the human body, with its materials and construction allowing for flexibility and adaptability to various body shapes and sizes. The points of contact between the wearable structure and the user's body are optimized to distribute pressure evenly, reducing the risk of discomfort or skin irritation.

The placement and operation of user interface elements, such as buttons, switches, or touch-sensitive areas, are designed to be easily accessible and intuitive to use. These interface elements are strategically located to prevent accidental activation or discomfort during movement.

In addition, the device incorporates features to reduce heat accumulation, such as heat dissipation mechanisms or materials, to maintain a comfortable temperature at the skin interface during use. The design also considers the acoustics of the device, ensuring that any operational sounds generated by the device (such as from the ultrasound transducer units) are within acceptable levels to prevent auditory discomfort.

The wearable structure of the ultrasound therapy device incorporates skin-friendly adhesives, designed to firmly affix the device to the user's body during therapy sessions. These adhesives secure the device in position, ensuring consistent contact between the ultrasound transducer units and the skin for effective delivery of the ultrasound therapy.

The adhesives are biocompatible and hypoallergenic, designed to minimize the risk of skin irritation or allergic reactions. They exhibit strong adhesive properties to keep the device securely attached, even during movement or perspiration, yet can be easily removed without causing pain or leaving residue on the skin.

For example, one possible adhesive choice could be medical-grade acrylic adhesives. Acrylic adhesives are commonly used in medical applications due to their high biocompatibility, strong adhesion properties, and resistance to moisture. These adhesives can be applied to the wearable structure in the form of adhesive patches or strips, which can be covered with a removable protective layer until use.

Another example could be silicone-based adhesives. Silicone adhesives are known for their skin-friendly properties, including painless removal, good adherence even to moist skin, and minimized risk of skin irritation. They could be incorporated into the design in a similar manner as acrylic adhesives.

In some embodiments, the device could utilize a reusable adhesive, possibly supplemented with an adhesive renewal solution. This would allow the user to clean and reactivate the adhesive surface multiple times, extending the usability of the device and reducing waste.

Furthermore, the device could be designed to have different areas with varying adhesive strength, depending on their position and function. For example, areas of the device around the ultrasound transducer units, where firm skin contact is crucial for effective therapy delivery, could have stronger adhesive properties than other parts.

The integration of skin-friendly adhesives into the wearable structure aids in maintaining proper placement and stability of the device during ultrasound therapy, enhancing therapeutic effectiveness without sacrificing user comfort.

The ultrasound therapy device incorporates various smart sensors that continuously monitor the positioning and fit of the wearable structure on the user's body. These sensors are intended to ensure optimal alignment of the ultrasound transducer units with the targeted therapy area and secure positioning of the device throughout the duration of the therapy session.

Positioning Sensors: Positioning sensors such as accelerometers, gyroscopes, and magnetometers can be used to detect the orientation and position of the device on the user's body. These sensors can work in unison to provide a comprehensive understanding of the device's alignment with respect to the therapy area.

Pressure Sensors: Pressure sensors can be integrated into the wearable structure to monitor the contact pressure between the ultrasound transducer units and the user's skin. Consistent and appropriate pressure is crucial for effective ultrasound therapy delivery. If the pressure deviates from an optimal range, the user can be alerted to adjust the device or the device could automatically adjust its position or pressure.

Skin Proximity Sensors: These sensors, potentially in the form of capacitive or infrared sensors, can be used to confirm the proximity of the ultrasound transducer units to the user's skin. If the sensors detect a gap or incorrect distance, they can alert the user or trigger an automatic adjustment mechanism in the device.

Temperature Sensors: These sensors can monitor the skin's temperature under the device to ensure the user's comfort and prevent overheating, which could lead to skin irritation or discomfort.

The data collected by these sensors can be processed in real-time or near real-time by the device's onboard processor, which can then provide immediate feedback to the user through visual, auditory, or haptic alerts. For instance, if the device detects it's misaligned, the user could be alerted by a blinking LED light or a gentle buzzing. In more advanced versions of the device, these sensors could interface with an automatic adjustment mechanism, which can autonomously correct the position or fit of the device.

The ultrasound therapy device includes easy-to-use tightening and pressure adjustment mechanisms to ensure a secure and comfortable fit of the wearable structure, as well as optimal therapeutic effectiveness.

Tightening Mechanism: This feature allows the user to adjust the tightness of the wearable structure around their body, ensuring a snug fit without causing discomfort. The tightening mechanism could be implemented in various ways, such as:
a. Adjustable Straps: These could be designed with hook and loop fasteners (e.g., Velcro), buckles, or a ratchet system. This allows the user to adjust the tightness manually to find the most comfortable and secure fit.
b. Elastic Material: Part of the wearable structure could be made from elastic materials which naturally conform to the user's body. This design can provide a level of automatic adjustment to body movements while maintaining a secure fit.

Pressure Adjustment Mechanism: This allows the user to alter the pressure applied by the ultrasound transducer units on the skin. The pressure adjustment mechanism can influence the intensity of the therapy and user comfort, and could be implemented in various ways, such as:
a. Mechanical Dial or Buttons: A dial or buttons could be included on the housing of the device, enabling the user to manually increase or decrease the pressure of the transducer units against their skin.
b. Pneumatic or Hydraulic Systems: These systems could adjust the pressure applied to the skin by inflating or deflating a component of the device, or altering fluid pressure in a compartment of the device. The user can control this mechanism through a simple control interface on the device or a connected app.

Both the tightening and pressure adjustment mechanisms are designed to be intuitive and user-friendly, minimizing the learning curve for the user. Visual indicators, such as color-coded levels, could be added to these mechanisms to provide a quick and easy reference for the user when making adjustments.

Some embodiments comprise an adaptive system that automatically adjusts ultrasound transmission based on a variety of parameters. This system could incorporate a combination of sensors and intelligent algorithms to evaluate the current conditions and optimally configure the ultrasound delivery. Examples of this may include but is not limited to an adaptive ultrasound transmission system, comprising sensors and a sophisticated processing algorithm that can automatically tune the device's output for the optimal therapeutic effect. Said sensors may comprise one or more sensors capable of measuring key parameters affecting ultrasound transmission, such as skin temperature, contact pressure between the transducer and skin, skin hydration level, and/or user feedback on comfort or effectiveness. It may further comprise a processing algorithm, designed to analyze the data received from the sensors in real-time or near real-time, taking into account the variability of these parameters and adapting the ultrasound delivery accordingly. The algorithm could adjust the frequency, intensity, duty cycle, and pulse shape of the ultrasound waves to ensure optimal therapy delivery based on conditions.

In one example, if the sensors detect a low level of contact pressure or high skin temperature, which might suggest a less efficient ultrasound transmission, the algorithm could automatically increase the ultrasound intensity or adjust the frequency to compensate. Conversely, if the contact pressure is high and skin temperature is low, indicating potentially more efficient transmission, the algorithm could reduce the intensity to prevent overexposure.

By continuously monitoring and adjusting to the changing conditions, the adaptive system can maintain optimal therapeutic efficacy and user comfort without the need for a separate step to test the expected throughput of the impedance matching material. This design could streamline the operation of the device, enhance user-friendliness, and improve overall therapeutic outcomes.

Some embodiments comprise a direct wired communication system. The direct wired communication system in this may comprise physical connections using wires or cables for transmitting signals between various components of the ultrasound therapy device. This could include transmission of power, control signals, sensor readings, and other necessary information for the operation of the device. This may further comprise inter-component communication: The ultrasound transducer units, energy source, and the processor could all be interconnected using physical wiring. This can ensure direct, reliable transmission of power and signals. For example, control signals from the processor to the transducer units could directly command the operation of the transducers (like starting/stopping the ultrasound emission, adjusting the frequency or intensity, etc.). The data from the sensors (like skin temperature, contact pressure, etc.) could be directly transmitted to the processor for analysis and action.
1. External Communication: For communication between the device and an external controller (such as a handheld remote control or a control interface on a smartphone), an option could be using a wired connection such as a USB cable. This would provide a reliable, direct connection for control signals and potentially also for charging the device's power source.
2. Alternative Wired Solutions: Another option could involve the use of advanced wired communication protocols like Ethernet or HDMI. These protocols can transmit a large amount of data very quickly and reliably, which could be useful for complex devices or for devices that need to communicate with external systems (such as a computer or a specialized medical device).

The present disclosure relates to an ultrasound therapy device designed to deliver preventative stroke therapy, and includes an arrangement of various integrated features and components to effectuate an anti-coagulating impact within the cardiovascular system of a user, particularly the heart.

In an embodiment, the device may be configured as a wearable therapeutic patch. The patch is constructed with lightweight, flexible, and user-friendly materials to ensure comfort and ease-of-use when affixed onto the chest area proximal to the user's heart. A primary component of the wearable patch is an array of ultrasound transducer units. These units are strategically located on the skin-facing side of the patch to ensure effective and targeted delivery of ultrasound therapy.

The main therapeutic function of the disclosed device lies in its ability to generate and emit shockwaves of predetermined frequencies and intensities. This therapy induces certain biochemical alterations within the bloodstream that serve to inhibit the formation of blood clots. Notably, the emitted ultrasound may stimulate the increase of nitric oxide levels within the blood, a condition known to prevent the aggregation of platelets and activate the process of fibrinolysis—a biological mechanism responsible for the dissolution of blood clots.

In an embodiment, the device would be administered to users with risk factors above average relative to the broader population. The risk of stroke increases with age, especially after 55 years. Older individuals are more likely to develop conditions such as high blood pressure, atherosclerosis, and atrial fibrillation, which can increase the risk of stroke. Some of the risk factors for stroke include:

Hypertension (High Blood Pressure): High blood pressure is a leading risk factor for stroke. It can damage blood vessels and lead to the formation of blood clots or weakened vessel walls, increasing the risk of stroke.

Diabetes: Individuals with diabetes have a higher risk of developing stroke due to the association with other risk factors such as high blood pressure, high cholesterol levels, and obesity.

Smoking: Smoking damages blood vessels, increases the formation of blood clots, and accelerates the development of atherosclerosis. These factors significantly raise the risk of stroke.

High Cholesterol: High levels of cholesterol contribute to the buildup of fatty deposits in the blood vessels, narrowing the arteries and increasing the risk of blood clots.

Atrial Fibrillation: Atrial fibrillation is an irregular heartbeat that can cause blood clots to form in the heart. If a clot travels to the brain, it can lead to a stroke.

Obesity and Sedentary Lifestyle: Being overweight or obese and leading a sedentary lifestyle can increase the risk of various conditions such as high blood pressure, diabetes, and heart disease, which in turn increase the risk of stroke.

Previous Stroke or Transient Ischemic Attack (TIA): Individuals who have previously experienced a stroke or TIA are at a higher risk of subsequent strokes.

Family History and Genetics: Having a family history of stroke or certain genetic conditions can predispose individuals to a higher risk of stroke.

Ethnicity: Some ethnic groups, such as African Americans, have a higher risk of stroke compared to other populations.

In some embodiments, an ultrasound therapy device is designed for the treatment of acute myocardial infarction (AMI). The device employs an array of features and integrated components to enhance blood flow and dilate arteries, thus mitigating the symptoms and potential damage associated with AMI.

In one embodiment, the device is constructed as a wearable therapeutic patch. This patch is designed to be both lightweight and flexible, providing user comfort when placed on the chest area, directly above the heart. The skin-facing side of the patch features an array of ultrasound transducer units, configured to effectively deliver therapeutic ultrasound waves to the target area.

The primary therapeutic objective of the disclosed device is to generate and direct shockwaves. The transducer units are controlled to emit these shockwaves at precise frequencies and intensities, based on the patient's unique physiological needs.

The treatment protocol administered by the device is designed to mitigate the impact of AMI. The emitted shockwaves act upon the vasculature in the target region, causing an increase in blood flow and the dilation of arteries. This increase in blood flow, coupled with the dilation of arteries, can significantly alleviate the symptoms of AMI and limit potential myocardial damage, thereby serving as an effective therapeutic strategy for patients suffering from acute myocardial infarction.

In some embodiments, an ultrasound therapy device is designed for post-myocardial infarction recovery. The device comprises a memory unit and a processor, wherein the processor is configured to control an array of ultrasound transducer units based on predefined therapeutic parameters stored in the memory unit.

The therapeutic parameters encompass ultrasound frequency, intensity, pulse duration, and temporal pattern of the ultrasound waves, and are specifically designed for promoting tissue regeneration and minimizing scar formation post-myocardial infarction. In one embodiment, the frequency of the ultrasound waves is within the range of 0.5 to 3 MHz, while the intensity is within the range of 0.5 to 2.0 W/cm².

Upon activation, the array of ultrasound transducer units generates ultrasound waves, which are directed towards the heart tissue of a user. These waves are configured to stimulate various cellular responses, including but not limited to the upregulation of growth factors, modulation of the immune response, stimulation of angiogenesis, recruitment and proliferation of cardiac stem cells and endothelial cells, and the remodeling of the extracellular matrix. These responses collectively contribute to enhanced regeneration of cardiac tissue and reduced formation of scar tissue post-myocardial infarction.

In some embodiments, the device comprises a suite of sensors configured to monitor one or more physiological parameters of the user. This suite of sensors may include but is not limited to sensors for heart rate, blood pressure, or other biomarkers. The data collected from these sensors may be utilized by the processor to adaptively adjust the therapeutic parameters of the ultrasound waves in real time, thereby optimizing the therapeutic outcomes for the user.

Therefore, these embodiments can provide a non-invasive, personalized approach to enhancing post-myocardial infarction recovery, by utilizing therapeutic ultrasound to promote tissue regeneration, minimize scar formation, and adaptively adjust therapy parameters based on the monitored physiological parameters of the user.

In an embodiment, a shockwave therapy device, configured as a vest, is described for alleviating pressure in the lungs and left side of the heart, as well as reducing interstitial fluid. The device comprises a wearable structure, an array of shockwave transducer units, a power source, a communication system, and a processor configured to control the operation of the transducer units.

The wearable structure of the device conforms to the shape of a user's torso, providing broad coverage over the chest area to facilitate therapy targeting the lungs and heart. The structure is crafted from a lightweight, flexible, and breathable material, ensuring user comfort during prolonged use.

The array of shockwave transducer units is incorporated within the wearable structure and is configured to generate shockwaves upon receiving a signal from the processor. The shockwaves are oriented towards the lungs and the left side of the heart, to effect therapeutic changes in these areas.

The shockwave therapy administered by the device is designed to alleviate pressure in the lungs and the left side of the heart. This therapy may include mechanical stimulation of the targeted areas, leading to dilation of blood vessels and bronchial passages, improved blood flow and oxygenation, and reduced pressure.

Additionally, the shockwaves can stimulate the lymphatic system, encouraging the reduction of interstitial fluid, and consequently decreasing edema and discomfort in the user. The device thus provides a non-invasive approach to pressure alleviation in individuals with conditions such as congestive heart failure, pulmonary hypertension, or pulmonary edema.

In some embodiments, the device includes sensors for monitoring physiological parameters of the user, such as heart rate, blood pressure, oxygen saturation, or lung fluid levels. The processor may utilize data from these sensors to adaptively adjust the intensity, frequency, and duration of the shockwaves in real-time or near real-time, optimizing therapeutic outcomes.

In conclusion, these embodiments provide a versatile and adaptive shockwave therapy vest designed to alleviate pressure in the lungs and heart, reduce interstitial fluid, and improve overall patient comfort and well-being.

In one embodiment, an ultrasound therapy device is designed to treat heart arrhythmias through neuromodulation via shockwave therapy. The device comprises a wearable structure, an array of shockwave transducer units, an energy source, a communication system, and a processing unit.

The wearable structure is designed to be comfortably worn on the user's chest, around the heart area. The structure incorporates lightweight, flexible, and skin-friendly materials to ensure user comfort during prolonged use. The device is configured to generate and direct shockwaves towards the heart to impact heart rhythms.

The array of shockwave transducer units, located within the wearable structure, is configured to generate shockwaves at frequencies and intensities suitable for neuromodulation. This shockwave therapy aims to modulate the activity of the cardiac autonomic nervous system, which controls heart rhythm, with a goal of rectifying irregular heart rhythms such as tachycardia, bradycardia, or atrial fibrillation.

The processing unit controls the operation of the shockwave transducer units, determining the frequency, intensity, and timing of the shockwaves based on the patient's specific therapeutic needs. The processor may adjust these parameters in real-time or near real-time based on feedback from integrated physiological sensors, enhancing the device's efficacy and safety.

In some embodiments, the device incorporates sensors for real-time or near real-time monitoring of the user's heart rhythm. This information can be utilized by the processor to adaptively adjust the shockwave parameters, providing an individualized and responsive therapy.

In yet another aspect, the device may include a user interface to provide feedback to the user about their heart rhythm and the ongoing treatment progress. The user interface may include visual, auditory, or haptic feedback mechanisms. In an embodiment, the therapy could be aimed at modulating the activity of the cardiac ANS to normalize heart rhythms by targeting specific nerves: The shockwaves could be directed towards specific nerve clusters associated with heart regulation, such as the cardiac plexus or vagus nerve. The goal would be to modulate the firing of these nerves, thereby influencing the heart's rhythm. For instance, enhancing vagal activity (parasympathetic) could help slow down a rapid heart rate (tachycardia). Varying shockwave parameters: The frequency, intensity, and duration of the shockwaves can be adjusted to influence different aspects of nerve function. For instance, low-frequency, short-duration shockwaves may stimulate nerve activity, while high-frequency, long-duration shockwaves may suppress it. The device's processing unit can adjust these parameters based on real-time or near real-time heart rhythm data. Synchronized therapy: The shockwaves could be synchronized with the patient's heart rhythm to achieve targeted neuromodulation. For instance, applying a shockwave at a specific phase of the cardiac cycle (e.g., during the refractory period after a heartbeat) may help prevent unwanted extra beats (premature ventricular contractions).

In some embodiments, the ultrasound waves are focused primarily on the spastic muscles in the lower limb, fostering neuromodulation that aims to reduce muscle spasticity. The physiological response induced by the ultrasound waves includes, but is not limited to, neural inhibition, muscle fiber relaxation, reduction in muscle tone, and suppression of excessive reflex activity. In such an embodiment, the wearable ultrasound therapy device may contribute to alleviating symptoms of lower limb spasticity in stroke patients, potentially improving their muscle function, mobility, and overall quality of life.

Some embodiments propose a therapeutic device tailored for the rehabilitation phase following a stroke. It can include an advanced, adaptive calibration mechanism in the processor, which is equipped to adjust the operating parameters of the ultrasound transducer units. These parameters, namely frequency, intensity, duty cycle, and duration of the ultrasound waves, are regulated to create an optimized therapeutic environment to promote neuroplasticity and neurogenesis in the brain. The ultrasound transducer units are configured to generate therapeutic ultrasound waves in a frequency range conducive to eliciting neuroplastic responses. The specific frequency and intensity parameters can vary, influenced by factors such as the patient's specific condition, the target area in the brain, and the current stage of the recovery process.

In another embodiment, the device is used on patients who are undergoing a cancer therapy-based chemotherapy regimen, including but not limited to doxorubicin (DOX)-based cancer therapies. In this context, the device offers a two-pronged therapeutic approach. First, through the application of low-intensity extracorporeal shockwaves, the device acts as a protective shield for the cardiomyocytes, the cells most vulnerable to cancer therapy-induced cardiotoxicity. These therapeutic shockwaves are generated at parameters specifically optimized for cardiomyocytes protection, thereby mitigating the detrimental impacts of cancer therapy on cardiac cells. When ESW therapy is applied to the cardiac area, it induces mechanical forces that activate mechanosensors in cardiomyocytes such as integrins αvβ and αvβ5, initiating a signaling pathway. After integrin-linked kinase (ILK) activation by the ESW therapy, ILK triggers the phosphorylation of Akt, which is a molecule involved in cell survival and anti-apoptotic pathways. This, in turn, leads to the upregulation of survivin which is an anti-apoptotic protein that inhibits cell death in cardiomyocytes. Chemotherapy downregulates and upregulates transcription factors for survivin expression (e.g. Sp1 and p53), while ESW therapy reverses the downregulation of Sp1 and the upregulation of p53 that could be caused by chemotherapy, resulting in the increased expression of survivin, consequently protecting the cells from apoptosis and thereby reducing the cardiotoxic effects of chemotherapy.

In addition to offering protective benefits, the device also contributes towards the therapeutic management of cancer therapy-induced cardiomyopathy. Through the judicious application of extracorporeal shockwaves, the device can stimulate biological pathways conducive to the healing and recovery of cardiomyocytes affected by cancer therapy-induced cardiomyopathy. The precise frequencies, intensities, and durations of the shockwaves can be programmed and tailored according to individual patient needs and clinical presentations. With the capacity to both protect against and treat cancer therapy-induced cardiotoxicity, the device effectively minimizes the cardiotoxic side effects associated with cancer therapy administration. This pioneering therapeutic approach ushers in a new paradigm for safer and more efficacious cancer treatment protocols. The device and methods herein, thus, mark a significant advancement in the field of oncology and cardiovascular medicine, offering a robust and non-invasive solution for reducing the cardiac risks associated with cancer therapy-based chemotherapy.

The present disclosure is directed to a wearable ultrasound therapy device designed to administer personalized extracorporeal shockwave therapy (ESWT) by utilizing real-time or near real-time data collection and analysis. The device is constructed with several key components, including an adaptable wearable structure, such as a Shoulder-Chest Harness with chest bands and shoulder straps, designed to fit the contour of a user's body.

The device features an array of lightweight, compact ultrasound transducer units, capable of generating low-intensity shockwaves. These transducer units are brought into contact with the user's body using various impedance matching materials, such as water-based gels, hydrogel matrixes, silicone-based gels or matrixes, or conductive gels, to facilitate effective penetration of the shockwaves.

A control unit is included, either attached to the wearable structure or as a separate handheld device. This unit encompasses a processor, memory, a power source unit, a failure alarm unit, and a user-friendly interface for data input and display. The interface allows for the entry of important sociodemographic data, medical history, and initial treatment parameters.

A plurality of physical and physiological sensors are incorporated into the device, including but not limited to devices for electrocardiography (ECG), seismocardiography (SCG), ballistocardiography (BCG), phonocardiography (PCG), bioimpedance measurement, heart rate monitoring, heart rate variability monitoring, blood pressure monitoring, oxygen saturation or pulse oximetry, as well as accelerometer, gyroscope, and magnetometer. The device also includes electrochemical immunosensors for analyzing body fluids (e.g., sweat, blood, urine) to track excess pharmacological components or other biomarkers indicative of cardiomyopathy, such as cardiac troponin and brain natriuretic peptide. Electrochemical sensors for the detection of molecules like lactate or glucose are also incorporated.

The device is designed to autonomously adjust ESWT parameters based on real-time or near real-time sensor readings, thereby providing personalized treatment. By directing shockwaves to the targeted heart area, the device aims to protect cardiomyocytes from cancer therapy-induced damage, thus opening new possibilities for mitigating the cardiotoxicity associated with cancer therapy administration. This innovative approach combines focused extracorporeal shockwave treatment with ongoing chemotherapy and/or radiation therapy to safeguard the heart health of cancer patients.

During its operation, the user or a healthcare professional can first input relevant sociodemographic information, medical history, and initial treatment parameters into the control unit's user interface. The device can then be positioned over the heart region, which has been pre-mapped during an initial calibration process. Subsequently, the device collects and analyzes current physiological information, administers shockwaves, and continues to monitor cardiac health parameters periodically before, during, and after each ESWT session and chemotherapy. As such, the device presents a holistic approach to protecting cardiomyocytes and minimizing the risk of cancer therapy-induced cardiomyopathies and/or congestive heart failure.

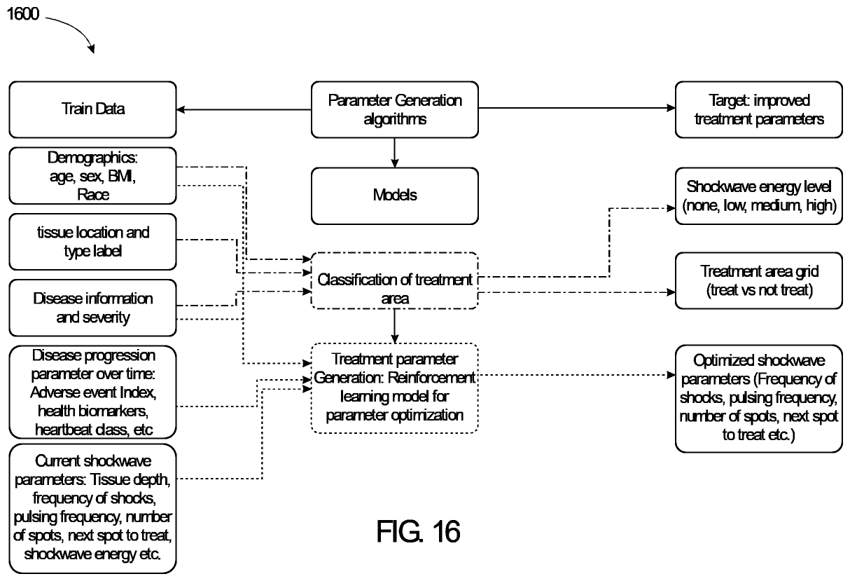

The present disclosure pertains to an adhesive patch variant of an ultrasound therapy device, FIG. 13. This compact device is designed to be affixed directly onto the skin of a user, thereby providing a more convenient and unobtrusive therapeutic solution.

The adhesive patch 1300 is made from hypoallergenic materials that comfortably adhere to the skin. It features an array of compact ultrasound transducer units embedded within the patch 1304. These transducer units are configured to generate low-intensity extracorporeal shockwaves for therapy. The generated shockwaves are facilitated through an impedance matching layer of material, such as hydrogel, incorporated within the patch. This material assists the effective penetration of the shockwaves into the user's body.

The device's control unit, consisting of a miniaturized processor, memory, and a power source, is embedded within the adhesive patch, making the device wholly self-contained. A user interface for data input and display may be incorporated into the device or be accessible via wireless communication with an external device, such as a smartphone or a tablet.

Integrated around the perimeter of the patch is an electrode ring designed to perform electrocardiography (ECG) 1302. This feature allows for the continuous monitoring of the user's heart rate and rhythm, providing essential cardiac health data.

The adhesive patch device is also equipped with a photoplethysmogram (PPG) sensor 1306. The PPG sensor, often used in pulse oximeters, employs a light source and a photodetector to measure the variation in blood volume with each heartbeat. This allows for the continuous monitoring of the user's blood oxygen saturation level and pulse rate, providing additional data for health monitoring.

The wearable ultrasound therapy device takes the form of a lightweight, flexible, and conformal adhesive patch designed for comfortable wear on the chest area. The adhesive patch has dimensions roughly corresponding to an average-sized adult human heart, to provide comprehensive coverage of the target treatment area.

The ultrasound transducer array is embedded into the adhesive patch and is composed of a multitude of miniature transducer units 1304. Each unit is controlled independently, allowing for phased array operation, and they are arranged in a grid-like pattern. This arrangement is designed to ensure even distribution of ultrasound waves and maximum treatment uniformity, with sufficient overlap to avoid treatment gaps. The array is also designed for electronic steering, allowing the device to focus ultrasound waves on specific areas of the heart without physically moving the patch.

Around the edges of the adhesive patch, an electrode ring is placed, forming an ECG sensor 1302. This ECG sensor detects electrical signals from the heart, providing continuous heart rhythm monitoring.

At the center of the patch, a photoplethysmography (PPG) sensor is embedded 1306. This sensor illuminates the skin and measures changes in light absorption, enabling the monitoring of blood volume changes in the microvascular bed of tissue and thereby tracking the user's heart rate.

The adhesive patch is constructed from biocompatible materials, ensuring minimal skin irritation with long-term use. The patch is designed for single-use, with a peel-off backing that exposes a skin-safe adhesive for easy application and removal.

A thin layer of conductive gel is incorporated into the skin-facing side of the patch, enhancing the contact between the skin and the transducer units, and improving the transmission of ultrasound waves into the body.

To manage the heat produced by the transducer units, the patch is embedded with micro-channels for liquid cooling. These channels circulate a cooling fluid around the transducer units, absorbing and dispersing the heat, and keeping the patch at a comfortable temperature for the wearer.

The adhesive patch is wirelessly connected to a control unit, which can adjust the amplitude and phase of the ultrasound waves produced by each transducer, process the data from the ECG and PPG sensors, and manage the cooling system. This control unit can be a separate handheld device or an integrated part of a smart device like a phone or tablet. The patch and control unit are powered by a lightweight, rechargeable battery pack.

The wearable ultrasound therapy device, designed as an adhesive patch, is equipped with a wireless communication module that allows it to interface with an external computing device. This device can be any handheld system with the necessary processing capabilities, such as a smartphone, tablet, or dedicated handheld control unit.

The external computing device serves several crucial functions in the operation of the wearable ultrasound therapy device. First, it acts as the user interface, providing a means for the user or healthcare provider to input treatment parameters and view real-time or near real-time feedback on the therapy's progress. The interface might include controls for setting the intensity and frequency of the ultrasound waves, initiating and ending treatment sessions, and adjusting other parameters as necessary.

Second, the external device processes the data collected by the patch. This includes readings from the ECG sensor 1302, the PPG sensor 1306, and any feedback from the ultrasound transducers themselves 1304. Advanced signal processing algorithms and machine learning models can be used to analyze these data in real-time or near real-time, identifying trends and patterns that may indicate the need for adjustment in the therapy.

Finally, the external device controls the operation of the wearable device based on the processed data and the inputted treatment parameters. It sends commands to the patch to adjust the phase and amplitude of the ultrasound waves produced by the transducer array, as well as commands to manage the patch's cooling system. It can also trigger alerts or alarms based on specific conditions or thresholds detected in the data.

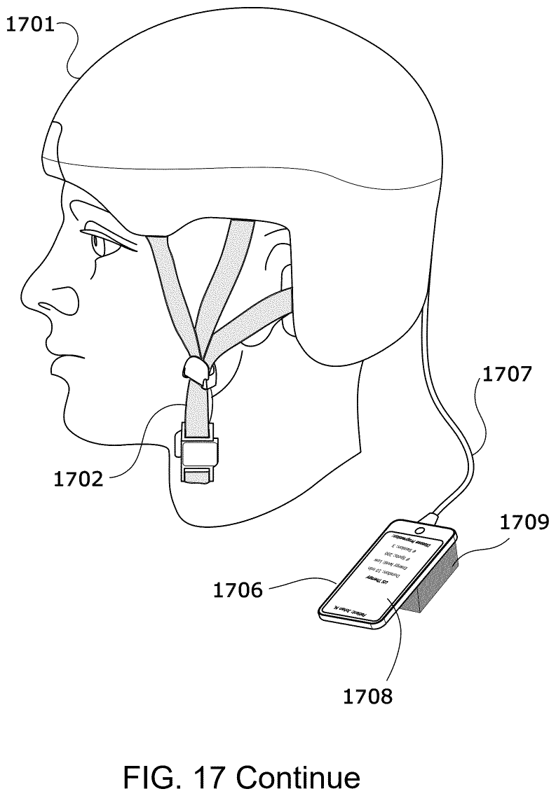

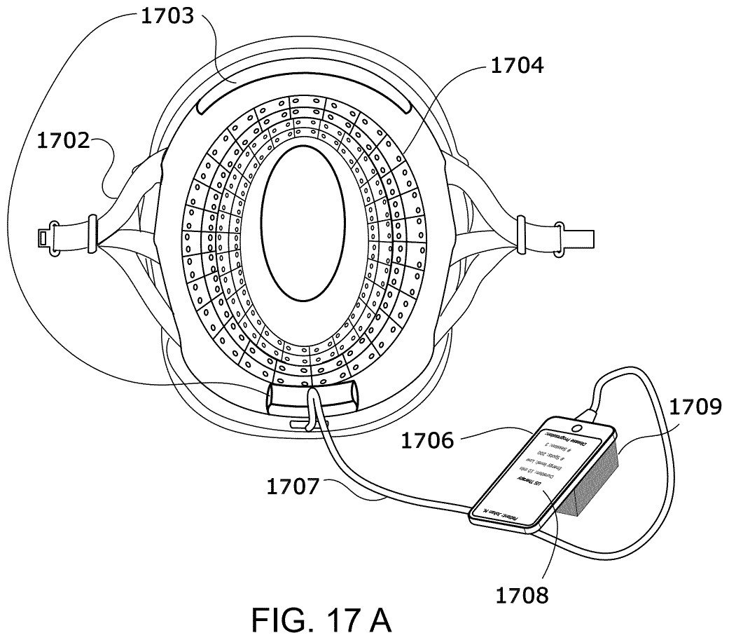

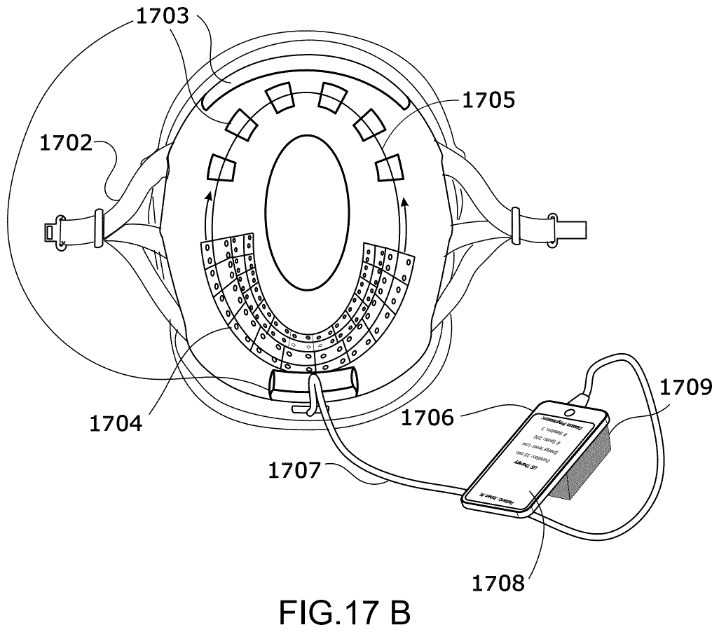

One embodiment includes an ultrasound therapy device specifically designed for the treatment of ischemic stroke and to facilitate improved drug delivery across the blood-brain barrier, FIG. 17. The device features a wearable helmet-like enclosure 1701 that is designed to conform to the shape of a user's head. This design ensures that the ultrasound waves are targeted to the desired locations within the brain. For added security and comfort, the helmet may include lateral stripes 1702 to secure it on the user's head and internal pads 1703. Within this helmet-like enclosure, there is a curved array of individual piezoelectric transducers 1704. These transducers are responsible for generating the focused ultrasound waves that provide the therapeutic effect. The array may be arranged in an annular configuration, FIG. 17A, optimizing the focusing and directing of the ultrasound waves. To enhance targeting precision, the device includes a mechanism for adjusting the positioning of the curved array 1705, FIG. 17B. There's an option for an automatic positioning and adjustment mechanism based on pre-programmed or real-time or near real-time inputs to ensure optimal delivery of the ultrasound waves.

The ultrasound therapy parameters could include aspects such as frequency, pulse repetition frequency, pulse duration, ultrasonic intensity, duty cycle, and therapy duration.

The control system of the device is responsible for adjusting the parameters of the ultrasound therapy. The control unit might employ machine learning algorithms to optimize these therapy parameters over time based on data from past therapy sessions. The device is also designed to provide a mechanical vibration and localized heating effect that temporarily disrupt the tight junctions between the endothelial cells in the blood-brain barrier, thereby increasing its permeability, enhancing drug delivery into the brain.

The external handheld system, 1706 is in communication with this control system 1707. This handheld system features a user interface and display 1708 that allows for controlling the ultrasound therapy device and for tracking therapy progress. The handheld unit can display a 3D model of the patient's brain and highlight the areas being treated in real time. Additionally, it might house a power supply or battery for the device, 1709.

For real-time or near real-time monitoring of the patient's condition, the device could include sensors such as temperature sensors, blood flow sensors, and other physiological sensors. This data could be vital for adjusting therapy parameters and ensuring the safety and efficacy of the treatment.

The device can have wireless communication capabilities, allowing it to share real-time or near real-time data with healthcare providers. This function enables remote monitoring of therapy sessions, which can be crucial in telemedicine and remote care scenarios. Finally, the patent covers a method of treating ischemic stroke or improving drug delivery using this device.

The method involves securing the helmet-like enclosure on the user's head, positioning the curved array of piezoelectric transducers, inputting therapy parameters into the handheld system's user interface, and then activating the array to generate the focused ultrasound waves and provide treatment.

The primary goals of treating an ischemic stroke are to restore blood flow and minimize brain cell death. Ultrasound therapy might be used to treat ischemic stroke through thrombolysis, to increase the effectiveness of clot-busting drugs (thrombolytics), such as tissue plasminogen activator (tPA), which can be administered in the case of an ischemic stroke to dissolve the blockage and restore blood flow. The combination of ultrasound and thrombolytic medication can potentially accelerate clot dissolution and improve patient outcomes. In this process, ultrasound waves are directed at the clot to help break it up and improve drug penetration.

Another potential treatment modality is sonothrombolysis whereby ultrasound is used without a clot-busting drug and used directly to break up the clot that is causing the stroke. The mechanical energy of the ultrasound waves causes the clot to break apart. Microbubbles (small gas-filled bubbles) can also be used to enhance the effect described in sonothrombolysis. When the ultrasound waves hit the microbubbles, they cause the bubbles to oscillate and implode, which helps to break up the clot.

Another potential application of ultrasound in stroke therapy is for neuroprotection, or the preservation of brain tissue following a stroke. Low-intensity ultrasound may have a protective effect on the brain after ischemia by inhibiting inflammation, reducing oxidative stress, and enhancing neuronal survival.

Opening the Blood-Brain Barrier (BBB) is another potential use case, whereby ultrasound, in conjunction with microbubbles, can be used to temporarily open the BBB, allowing for the delivery of neuroprotective and regenerative drugs to the brain. This could potentially enhance the treatment of ischemic stroke by enabling the delivery of therapies directly to the brain.

Another treatment modality may be neurorehabilitation through post-stroke rehabilitation through low-intensity pulsed ultrasound to enhance neuroplasticity and improving recovery after stroke. The application of ultrasound could potentially stimulate the growth and repair of brain cells and neural networks, supporting stroke recovery. Ultrasound employs sound waves at a frequency above human hearing capability. These waves can penetrate soft tissues and interact with them in multiple ways, including but not limited to thermal effect, causing vibration in tissues, generating heat as a result. This can help improve blood flow and reduce muscle tension.

Mechanical/cavitation is another potential effect, whereby ultrasound can cause a mechanical effect known as cavitation, where microbubbles are formed and collapsed in the tissue. This results in microstreaming around the bubbles, which can gently stretch cell membranes and enhance their permeability, promoting the movement of substances across cell membranes.

In the context of ischemic stroke, the biological outcomes or therapeutic effects of these physical mechanisms can include: thrombolysis and sonothrombolysis. Ultrasound can enhance clot-busting drugs' effectiveness or even break up clots on its own, thereby restoring blood flow to the brain, reducing the area of brain tissue death, and potentially improving patient outcomes. Ultrasound therapy could protect brain tissue from ischemic damage by reducing inflammation and oxidative stress and enhancing neuronal survival. Ultrasound therapy, particularly in combination with microbubbles, can help deliver drugs across the blood-brain barrier, allowing for more effective treatment. Post-stroke, ultrasound therapy might help enhance neuroplasticity and aid in the recovery process by stimulating the growth and repair of brain cells and neural networks. Ultrasound therapy might help to reduce brain swelling or edema, which is a common and dangerous complication of stroke. The heat generated by ultrasound can improve blood flow and help dissipate fluid buildup.

In summary, the therapeutic effects of ultrasound in the treatment of ischemic stroke are diverse, encompassing direct effects on the stroke (thrombolysis and sonothrombolysis), protection of brain tissue (neuroprotection), enhancement of drug delivery, and support for post-stroke recovery (neurorehabilitation).

In an embodiment, the ultrasound therapy takes the form of extracorporeal shock wave therapy (ESWT) to promote one or more of angiogenesis, the formation of new blood vessels and restoring blood flow to the brain. ESWT may stimulate angiogenesis through the activation of various growth factors, like vascular endothelial growth factor (VEGF), and endothelial nitric oxide synthase (eNOS). It may further be focused on promoting neuroprotection, neuronal regeneration and reducing neuronal cell death after a stroke. Furthermore, it may stimulate neurogenesis, the growth and development of new nerve cells, which can contribute to functional recovery after a stroke. Therapy may be focused on anti-inflammatory effects, to modulate inflammatory responses, which could be beneficial in the context of stroke, where inflammation can exacerbate injury. The therapy may also be focused on the improvement of motor function and recovery, to improve motor function and aid in the recovery process in stroke patients, reducing scar tissue, to help break down scar tissue and stimulate the healing and regeneration process, enhance drug delivery to enhance the delivery of therapeutics across the blood-brain barrier.

Some embodiments pertain to an ultrasound therapy device specifically designed for treating dementia by focusing on the hippocampus and disrupting amyloid plaques, thereby stimulating neurogenesis. The foundation of the therapy involves the use of focused ultrasound waves that can permeate the skull and reach targeted areas within the brain, while sparing surrounding tissue.

This ultrasound therapy device is unique in its strategic focus on the hippocampus, a region of the brain that is particularly impacted by dementia and crucial for memory and learning. The device employs a curved array of individual piezoelectric transducers housed within a helmet-like enclosure. These transducers produce focused ultrasound waves, with their focal point strategically oriented towards the hippocampus.

Central to the therapeutic effect is the disruption of amyloid plaques, which are protein aggregates commonly observed in the brains of dementia patients. The focused ultrasound waves generated by the device create mechanical vibrations that agitate and disrupt these plaques. This disruption is designed to alleviate some of the pathological effects of dementia and potentially slow the progression of the disease.

The therapy may additionally stimulate neurogenesis, the growth and development of new neuron cells as a therapeutic approach for dementia treatment. The device targets this potential by focusing ultrasound waves on regions where neurogenesis can occur, such as the hippocampus.

Some embodiments relate to an ultrasound therapy device for the treatment of neurological conditions such as ischemic stroke, dementia, Alzheimer's, Parkinson's, traumatic brain injury, and/or improved drug delivery across the blood-brain barrier, the device comprising:
a head-adaptable system, including a conformal array of individual piezoelectric transducers, each transducer being capable of individually adjustable operation for focused ultrasound waves emission towards a target site within the patient's brain;
a coupling pad configured to enhance acoustic coupling between the array of transducers and the patient's head, while adapting to its contour;
an integrated adjustment mechanism enabling real-time or near real-time manual and automatic positioning of the array of transducers based on a combination of patient-specific anatomical data and real-time or near real-time;
a sophisticated control system capable of autonomously and dynamically adjusting parameters of the ultrasound therapy, including but not limited to frequency, pulse duration, and intensity, based on real-time or near real-time feedback from monitoring sensors, patient-specific data, and predictive machine learning algorithms designed to optimize therapy efficacy, while ensuring low power consumption;
a portable handheld system with a user interface in communication with the control system, enabling healthcare professionals to set therapy parameters, monitor treatment progress in real-time or near real-time, and visualize treatment areas using three-dimensional renderings of the patient's brain.

In an embodiment, said head-adaptable system is not limited to any specific design or form, including but not limited to a helmet-like enclosure, headband, or stationary device, and can be configured to have an ergonomic design with features to secure the device to the patient's head.

The ultrasound therapy device may be capable of providing a mechanical vibration and localized heating effect to disrupt the tight junctions of the blood-brain barrier, thereby temporarily increasing its permeability, facilitating enhanced therapeutic drug delivery.

The external handheld system or any other user interface device may comprise a power source for the device, and can provide real-time or near real-time visualizations of the treatment area, including but not limited to 2D, 3D, or 4D imaging.

The ultrasound therapy device may further comprise sensors of any type to monitor the patient's condition during therapy, including but not limited to temperature sensors, blood flow sensors, physiological sensors, or any other relevant sensor technology.

The ultrasound therapy device may further comprise capabilities to facilitate communication with healthcare providers, including but not limited to wireless communication capabilities, and the ability to share treatment data in real-time or near real-time.

An example method of treating neurological conditions uses the ultrasound therapy device described above, the method not being limited to specific steps but involving the placement of the head-adaptable system, positioning the array of transducers, setting the therapy parameters, and activating the transducers to emit focused ultrasound waves.

Neurodegenerative diseases such as Parkinson's and Huntington's disease are characterized by the progressive loss of structure or function of neurons, which eventually leads to their death. These conditions can significantly affect a patient's quality of life as they progress, and there is a growing interest in using ultrasound as a non-invasive therapeutic approach for these diseases.

The ultrasound therapy device described here could potentially be adapted to treat such conditions in several ways. Neuromodulation through ultrasound therapy which can be used to target specific regions of the brain to induce neuromodulation, i.e., change the activities of neurons. This could potentially relieve symptoms in diseases like Parkinson's, where certain brain regions might be overactive or underactive. Stimulation of neurogenesis through ultrasonic stimulation of the production of brain-derived neurotrophic factor (BDNF), a protein that supports the survival of existing neurons and encourages the growth and differentiation of new neurons and synapses. Increased levels of BDNF could potentially slow the progression of neurodegenerative diseases.

The wearable device may utilize the cardiac rhythm detection sensors to implement cardiac gating procedures. Cardiac gating is a technique that synchronizes the delivery of ultrasound therapy with the patient's cardiac cycle. This synchronization ensures that the therapeutic shockwaves are delivered at the optimal time during the heart's rhythm, minimizing potential damage to the heart tissue and maximizing therapeutic effect.

The cardiac gating protocols can vary based on patient needs and the specific therapeutic application. Prospective high-pitch dual-source protocols involve rapid data acquisition in coordination with the cardiac cycle, which can reduce the exposure to radiation and shorten the overall scan time. Prospective step and shoot protocols involve pausing the data acquisition during certain parts of the cardiac cycle, which can reduce radiation dose and allow for high-resolution imaging. Retrospective gated helical protocols involve continuously acquiring data throughout the cardiac cycle, which can provide more comprehensive information about cardiac function but may involve higher radiation doses.

One common example of cardiac gating is ECG-based R-wave gating. In this protocol, the delivery of ultrasound therapy is synchronized with the R-wave of the patient's ECG, which corresponds to the ventricular contraction or the “beat” of the heart. This synchronization ensures that the therapeutic shockwaves are delivered when the heart muscle is contracting, which can optimize the therapeutic effect and minimize potential harm to the heart tissue.

Moreover, cardiac gating can be crucial for detecting and responding to adverse cardiac events. Any irregularities in the ECG, PPG, MCG, SCG, PCG, or echocardiography sensors' data can be indications of conditions such as heart arrhythmias, acute coronary syndromes, acute heart failure, pulmonary health conditions, embolisms, strokes, etc. By continuously monitoring the cardiac rhythm, the device can provide real-time or near real-time feedback and adjust the treatment parameters to optimize the therapeutic effect and respond to any adverse events.

In conclusion, the described wearable device, equipped with cardiac rhythm detection sensors and sophisticated cardiac gating protocols, represents a comprehensive, adaptive approach to ultrasound therapy, offering personalized, timely, and effective treatment for various cardiac conditions.

In addition to cardiac rhythm detection sensors, the wearable device may also incorporate breath detection sensors. These sensors can detect the user's breathing patterns, characterized by the inhalation and exhalation cycles, and use this information to further optimize the delivery of shockwave therapy.

Breath detection can be achieved by configuring the shockwave transducer units to listen for the characteristic sounds produced during the process of breathing. As air moves into and out of the lungs, it generates unique acoustic signals which these transducer units can interpret and analyze.

The data collected from the breath detection sensors allows for the implementation of breath-gating techniques. Breath-gating is a method where the delivery of shockwave therapy is synchronized with the user's breathing cycle. This synchronization can help to minimize treatment disruptions caused by the natural movement of the body during the breathing process.

For example, breath-gating could ensure that the shockwave therapy is administered during the exhalation phase of the breathing cycle, when the body tends to be more still and relaxed, or when the lungs are less filled with air, providing less interference for the therapeutic waves. This approach can help to ensure a more controlled and precise delivery of shockwave therapy, enhancing the overall efficacy of the treatment.

Furthermore, breath-gating techniques can also be beneficial in cases where patient movement might be detrimental to the therapy's effectiveness. For instance, in treatments targeting the heart or lungs, any displacement due to heavy breathing could potentially affect the treatment area or the quality of the ultrasound wave transmission. By synchronizing the shockwave therapy with the patient's breath cycle, the device can compensate for this movement, ensuring consistent and precise treatment delivery.

In summary, by incorporating both cardiac rhythm and breath detection sensors and gating techniques, the wearable device can provide a highly adaptable and tailored approach to ultrasound therapy, improving the precision, effectiveness, and safety of the treatment.

Some embodiments relate to a system and method for improving interventional cardiovascular therapies, particularly valvular interventions, aortic repair, and stent placements, by implementing shockwave therapy.

The system, herein referred to as the Shockwave Enhanced Interventional Therapy System (SEITS), incorporates a device capable of delivering focused, low-intensity shockwaves synchronously with standard cardiovascular procedures.

In an embodiment, SEITS is used to augment a valvular intervention. For instance, consider a procedure involving the placement of a transcatheter aortic valve replacement (TAVR). Typically, calcification of the valve can make this procedure challenging, leading to less than optimal outcomes. The SEITS is utilized to emit targeted shockwaves at the calcified aortic valve leaflets prior to and during the valve placement. These shockwaves serve to fracture and disrupt the calcified deposits, allowing for easier placement and better seating of the TAVR device, thus enhancing the overall success rate of the procedure.

In another embodiment, SEITS is utilized in aortic repair, particularly in the treatment of aneurysms. The shockwaves delivered by SEITS to the aortic walls can induce biological responses that promote the strengthening and remodeling of the aortic tissue. This may lead to a reduction in the diameter of the aneurysm and decrease the risk of rupture.

Moreover, in the case of endovascular stent placements, SEITS can be employed to improve outcomes. Restenosis, the re-narrowing of the artery after stent placement due to neointimal hyperplasia, remains a common issue. The shockwaves from SEITS can be applied during and after stent deployment. These shockwaves are hypothesized to modulate the vascular response, limiting the proliferation of smooth muscle cells and reducing the risk of restenosis.

The shockwave delivery of the SEITS is controlled by a processing unit. The processing unit synchronizes the shockwaves with the patient's cardiac cycle using real-time or near real-time electrocardiogram (ECG) data, thus ensuring shockwave delivery occurs during diastole when coronary blood flow is maximal. This reduces the risk of coronary ischemia during therapy.

Furthermore, the SEITS is equipped with an imaging system, such as intravascular ultrasound (IVUS) or optical coherence tomography (OCT), to provide real-time or near real-time visualization of the procedure. This ensures the accurate targeting of shockwaves and monitoring of therapeutic effects.

In conclusion, the SEITS provides a novel, integrative approach for improving the effectiveness of interventional cardiovascular therapies. By using shockwave therapy, the SEITS addresses common challenges associated with valvular interventions, aortic repair, and stent placements, enhancing therapeutic outcomes and patient prognosis.

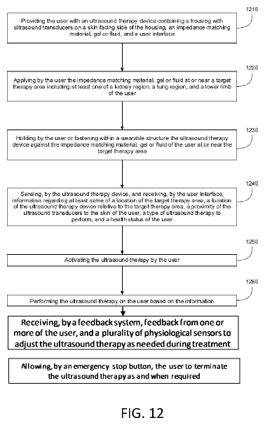

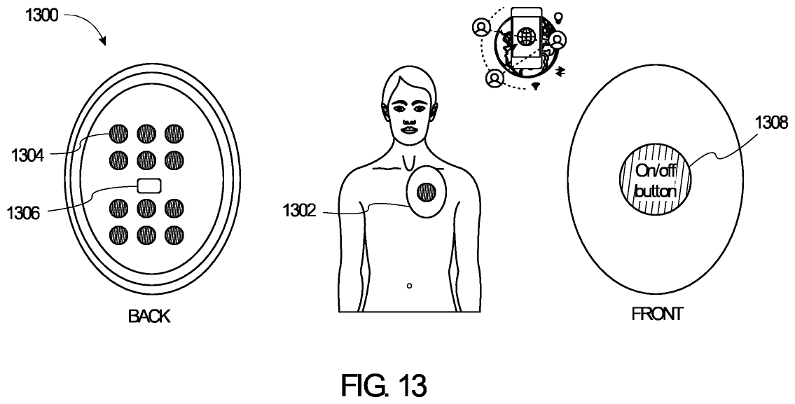

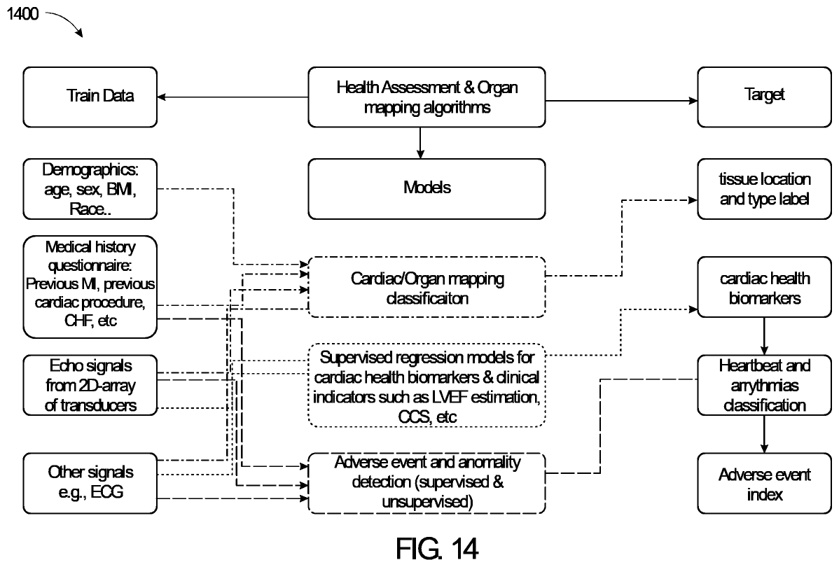

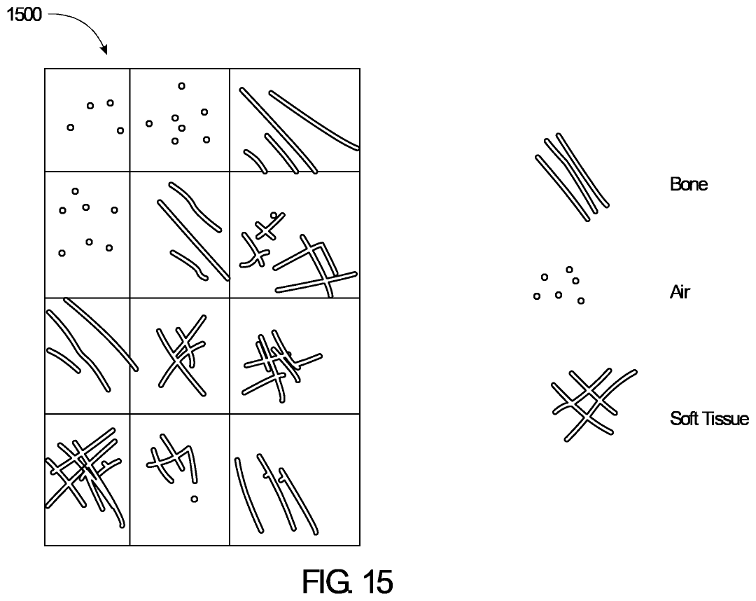

FIG. 12 is a flowchart of an example method for applying ultrasound therapy by a user. At step 1210, the user is provided with an ultrasound therapy device containing a housing with ultrasound transducers on a skin facing side of the housing, an ultrasound propagation medium, and a user interface. At step 1220, the user applies the impedance matching material, gel, or fluid 300 at or near a target therapy area including at least one of a kidney region, a lung region, and a lower limb of the user. As mentioned above, this impedance matching material, gel, or fluid 300 could be in the form of ultrasound gel or an ultrasound gel patch or water. At step 1230, either the user holds or wears and fastens a wearable structure that secures the ultrasound therapy device against the ultrasound propagation medium of the user at or near the target therapy area. At step 1240, information regarding at least some of a location of the target therapy area, a location of the ultrasound therapy device relative to the target therapy area, a proximity of the ultrasound transducers to the skin of the user, a type of ultrasound therapy to perform, and a health status of the user is sensed, by the ultrasound therapy device, and/or received, by the user interface. At step 1250, the user activates the ultrasound therapy. At step 1260, the ultrasound therapy device performs the ultrasound therapy on the user based on the information. At step 1270, the feedback system receives feedback from one or more of the user, and a plurality of physiological sensors to adjust the ultrasound therapy as needed during treatment. At step 1280, the emergency stop button allows the user to terminate the ultrasound therapy as and when required. Examples of the target therapy area include but are not limited to kidney region, a lung region, a lower limb region, a neck region, a shoulder region, an upper limb region, an abdominal region, and a back region of the user.

The present disclosure further relates to a device configured to provide an automated ultrasound treatment. The user can have access to the sensor data, such as ultrasonic data and/or electronic stethoscope data (and/or other minimally invasive health data of the user) from a plurality of regions of the user. The user can therefore identify the one or more diseases and can analyze the severity of said one or more diseases in one or more regions. This can be done by employing a disease type and severity machine learning model, e.g., classification model trained on clinical gold standards of different health conditions. E.g. a breathing test for pulmonary health, a d-dimer and/or ultrasound assessment of presence of thrombus, kidney function tests such as Glomerular filtration rate (GFR) and peripheral arterial blood flow tests such as an ankle-brachial index and/or a patient self-reported health outcome/wellbeing questionnaire, etc. These tests are not limited to preexisting tests but may comprise any measurements that indicate the efficacy of the ultrasound therapy. Then the user can identify the ultrasound therapy parameters (intensity, duration, and/or pulsation frequency) in one or more regions based on the user's characteristics, e.g. body size, age, type, and/or severity of disease (e.g. tissue stiffness may require an ultrasound therapy to relax the muscle and presence of thrombus may require an ultrasound therapy to destroy thrombus). Lastly, the user can administer the ultrasound therapy based on identified therapy needs.

For the first time usage of the automatic calibration of the ultrasound therapy device to map the location and size of the user's organs the user typically performs various steps. The user can access data of ultrasound sensor data and/or minimally invasive sensor 134 data from a plurality of regions (the data collection regions can be randomized and/or pre-set). Further, the user can identify the unique markers and/or patterns of each region for example by training a machine learning model to identify each region. Preferably, clustering methods can be used to segment data into different groups/regions. Furthermore, the position data corresponding to positioning of the sensors and/or ultrasound transducer unit for each region can be stored in memory and/or cloud so that data can be accessed at a later point.

For the first time usage, for manually calibrating the ultrasound therapy device to map the location and size of the user's organ, the user may perform various steps. Based on user characteristics (e.g., gender and body size), the device can be navigated to the desired region by a preset formula/decision rule. For example, if the user is female, 55 kg, and 70 years of age—one may position the ultrasound transducer unit in the bottom right quadrant of the device when the disease to treat is located in that region relative to the position of the device.

FIG. 13 illustrates a perspective view 1300 of the wearable ultrasound therapy device that acts as a chest-worn adhesive patch 1302, in accordance with one embodiment of the present disclosure. The wearable ultrasound therapy device takes the form of a lightweight, flexible, and conformal chest-worn adhesive patch 1302 designed for comfortable wear on the chest area. The chest-worn adhesive patch 1302 has dimensions roughly corresponding to an average-sized adult human heart, to provide comprehensive coverage of the target treatment area. The chest-worn adhesive patch 1302 has a front surface that includes an on/off button 1308 and a back surface that includes an ultrasound transducer array 1304. The ultrasound transducer array 1304 is embedded into the adhesive patch and is composed of a multitude of miniature transducer units. Each unit is controlled independently, allowing for phased array operation, and they are arranged in a grid-like pattern. This arrangement is designed to ensure an even distribution of ultrasound waves and maximum treatment uniformity, with sufficient overlap to avoid treatment gaps. The array is also designed for electronic steering, allowing the device to focus ultrasound waves on specific areas of the heart without physically moving the patch. Around the edges of the adhesive patch, an electrode ring is placed, forming an ECG sensor. This ECG sensor detects electrical signals from the heart, providing continuous heart rhythm monitoring.

At the center of the patch, a photoplethysmography (PPG) sensor 1306 is embedded. This PPG sensor 1306 illuminates the skin and measures changes in light absorption, enabling the monitoring of blood volume changes in the microvascular bed of tissue and thereby tracking the user's heart rate.

The chest-worn adhesive patch 1302 is constructed from biocompatible materials, ensuring minimal skin irritation with long-term use. The chest-worn adhesive patch 1302 is designed for single use, with a peel-off backing that exposes a skin-safe adhesive for easy application and removal.

A thin layer of conductive gel is incorporated into the skin-facing side of the patch, enhancing the contact between the skin and the transducer units, and improving the transmission of ultrasound waves into the body.

To manage the heat produced by the transducer units, the patch is embedded with micro-channels for liquid cooling. These channels circulate a cooling fluid around the transducer units, absorbing and dispersing the heat, and keeping the patch at a comfortable temperature for the wearer.

The chest-worn adhesive patch 1302 is wirelessly connected to a control unit, which can adjust the amplitude and phase of the ultrasound waves produced by each transducer, process the data from the ECG and PPG sensors, and manage the cooling system. This control unit can be a separate handheld device or an integrated part of a smart device like a phone or tablet. The patch and control unit are powered by a lightweight, rechargeable battery pack.

Further, the present description describes a set of machine-learning algorithms that can be divided into two categories: 1) a health assessment and organ mapping model; and 2) a personalized ultrasound therapy optimization system that utilizes patient-specific data to optimize ultrasound therapy parameters.

FIG. 14 illustrates a flow diagram 1400 related to cardiac-specific organ mapping and cardiac-specific health assessment models, in accordance with one embodiment of the present disclosure. The organ mapping and health assessment algorithm utilizes patient-related information such as but not limited to demographic data, medical history, and device-specific sensed signals from non-invasive sensors. The device-specific features are extracted from raw echo and other sensing signals data (e.g., ECG) using dimensionality-reducing techniques, such as but not limited to truncated discrete Fourier transform. During training, data augmentation such as but not limited to sampling across time and adding noise, is used to prevent overfitting and reduce variability across the time dimension. The ML models' cost function includes regularization components and other model-architecture components such as dropout layers in Convolutional Neural Networks for the echo data. Prediction post-processing is used to reduce more variable real-time or near real-time estimations to a more robust representation, such as a mean and a confidence interval based on estimations aggregated across time.

The organ mapping and health assessment algorithm comprises an organ mapping model that uses ultrasound-based signals from the 2D-array transducer as well as other sensing signals such as ECG for the cardiac region to map a grid of the organ region. In the grid mapping, a signal is sent at a certain depth and a 2D grid is created to estimate the locations of the organ (e.g., heart), unwanted tissues such as bone structure will be approximated on the grid to help optimize treatment locations. The cardiac mapping model can describe a classification problem where tissue type is classified (e.g., soft tissue, bone, air) in each location on the grid. The grid resolution is decided based on the treatment type, organ, and therapy area.

The method of mapping out a sub-skin layout involves using the ultrasound transducers or other sensors on the device to identify and/or visualize the internal structures of the tissues beneath the skin surface. The sub-skin refers to the region that lies beneath the skin surface, including layers of the skin (epidermis and dermis), and the associated fat layers (subcutaneous tissue) within the target area, and denotes the specific arrangements of the organs, tissues and anatomical features within the area. This mapping helps create a detailed representation or layout of the target area, providing information such as but not limited to dimensions and spatial distribution of the underlying tissues.

FIG. 15 illustrates a perspective view 1500 of the cardiac or organ mapping classification of a grid, in accordance with one embodiment of the present disclosure.

The health assessment models depict a supervised regression model to estimate health biomarkers and disease progression status (e.g., left ventricle ejection fraction LVEF for cardiac assessment). This model uses sensed signals from non-invasive sensors (e.g., 2D echo signal and ECG for cardiac monitoring). The health assessment models further depict classification supervised/unsupervised for adverse events and anomaly detection. For example, in the cardiac health assessment, a heartbeat and arrythmia class can be estimated from ECG input data. Additionally, the health assessment models depict target data estimated from the ML models that are combined to generate an adverse events index, defined to describe both the health and disease progression status (e.g., in cardiac application adverse event index is defined from heartbeat class and LVEF).

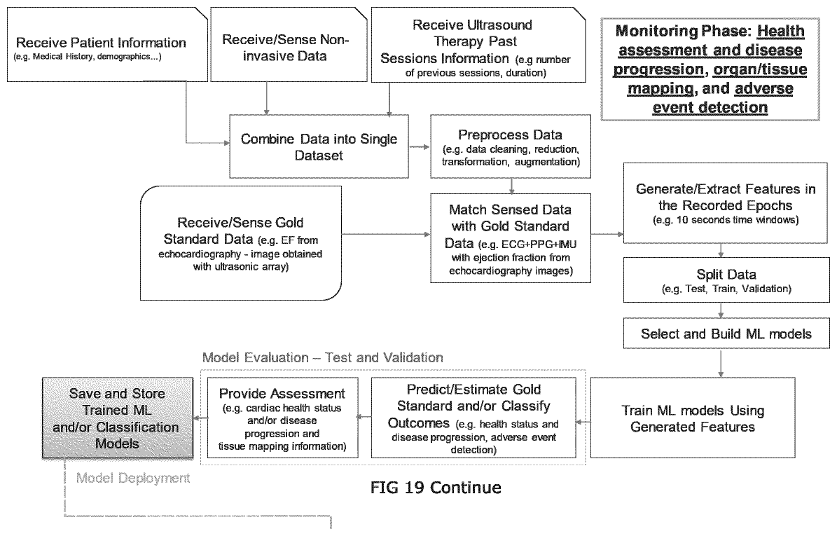

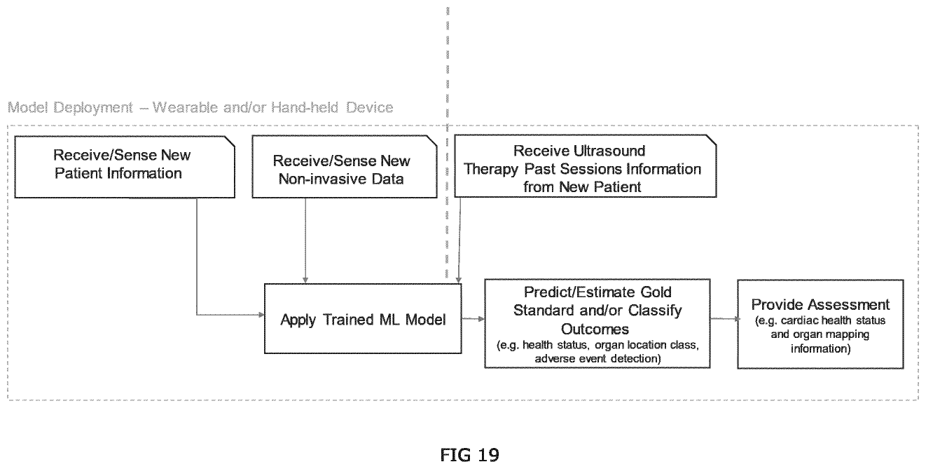

FIG. 19 depicts a general flow diagram of the development of the machine learning models involved in the health monitoring phase. The organ mapping and health assessment algorithm utilizes received patient-related information such as but not limited to demographic data, medical history, and received device-specific sensed signals from non-invasive sensors, as well as information related to previous ultrasound therapy sessions. The collected datasets are combined into a single dataset. This integration allows for the correlation of the three initial datasets, leading to the features extraction and generation from the integrated dataset. These extracted features work as input variables for the subsequent machine learning model training phase, to be able to provide an assessment on health status and disease progression, and an adverse event index. After training, testing and validation, the model is saved and stored on the device for future use.

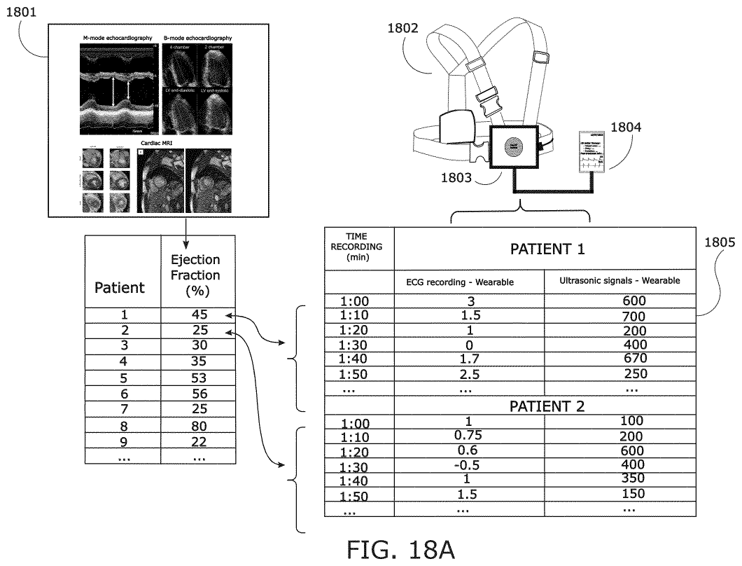

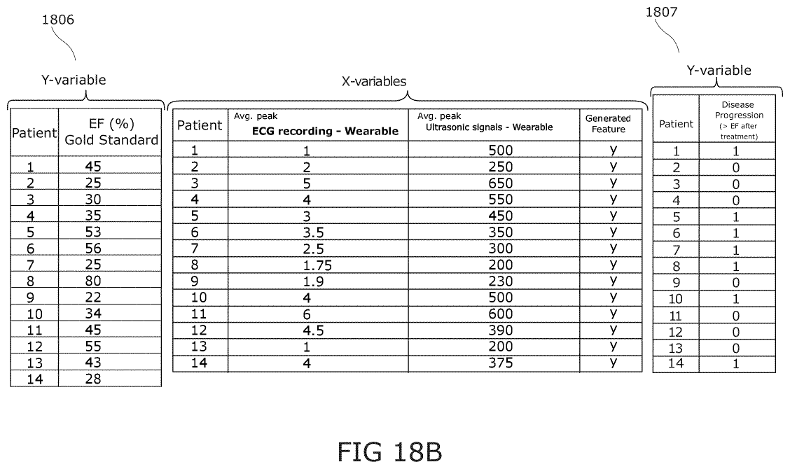

FIG. 18A shows a schematic example of the data matching step in the cardiac health assessment models. First, measured gold standard ejection fraction measurements from medical imaging techniques (e.g cardiac MRI or echocardiography) are collected 1801, and non-invasive signals (ECG and ultrasound echo signals) from the built-in sensors on the presented ultrasound therapy wearable device 1802 are recorded 1804, monitoring cardiac health for temporal windows (e.g. 10 seconds) 1805. In FIG. 18B, a pattern matching the behavior of the physiological data recorded from non-invasive sensors is matched to the gold standard, and new features are generated 1806. This allows for the model to assess cardiac health status of the patients and/or disease progression, based on an improvement of the recorded signals and observed patterns indicating a better ejection fraction 1807.

The ultrasound-based therapy optimization system can collect three types of data: patient-related information such as but not limited to demographic data, medical history, and sensed signals from non-invasive sensors, health outcome data/disease progression data recorded over time as a measure of treatment effectiveness such as but not limited to information recorded after an administered ultrasound-based therapy (e.g. Left ventricle ejection fraction or myocardial perfusion index improvement), and ultrasound therapy-related parameter information including but not limited to the periodicity of administration to keep track of how often the therapy takes place, the targeted therapy area and its dimensions (e.g. depth, and area), and therapy-specific parameters such as but not limited to frequencies, intensity/power (temporal average), ultrasound mode, duty cycle, and therapy duration.

FIG. 16 illustrates a block diagram 1600 of a cardiac-specific treatment therapy optimization system, in accordance with one embodiment of the present disclosure. The ultrasound-based therapy optimization system includes an initial (primary) treatment zone classification model. The model utilizes the organ mapping grid information obtained for the organ mapping model, patient demographics, and disease information to map the treatment classes to be administered on the grid region (treat vs no-treat). If treatment is specified an energy class is chosen based on the location map. The ultrasound-based therapy optimization system further includes a treatment parameters generation model using reinforcement learning (RL) which is a feedback-based ML, technique that follows disease progression and modifies treatment parameters accordingly.

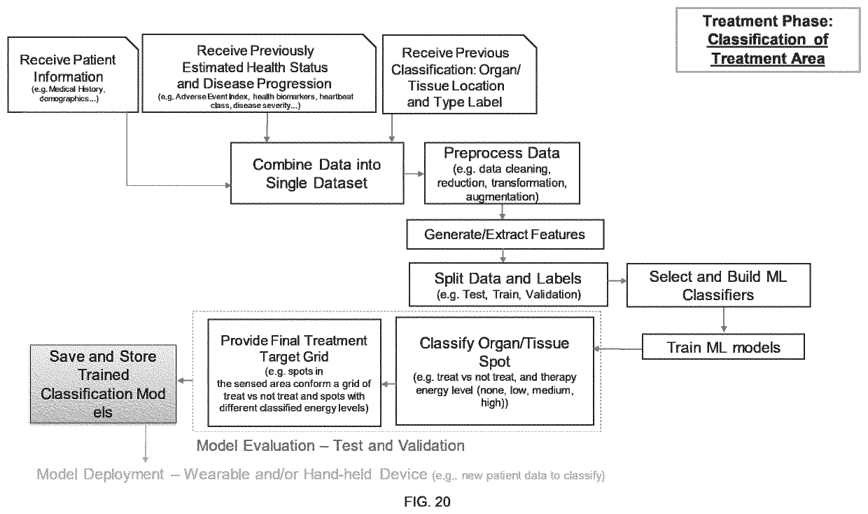

FIG. 20 depicts a general pipeline of the development of the classifier to initially classify the treatment area during the treatment phase. This machine learning model utilizes patient information (e.g. medical history and demographics), received disease information and estimated health status from previous health assessment models, and organ mapping information obtained from the organ mapping model in the previous health assessment phase. After the combination of the received data into a single dataset and preprocessing of the dataset has been completed, features are generated and the classifier is trained to produce a final grid with spots in the sensed area, confirm a grid to treat or not treat and provide information on classified energy levels (e.g. none, low, medium, high) of the spots.

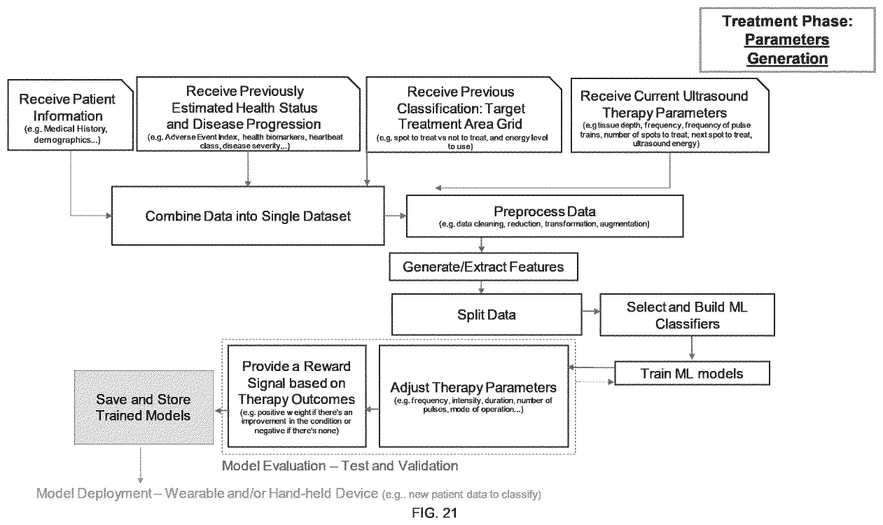

FIG. 21 depicts a general pipeline of the development of the treatment parameters generation model using reinforcement learning (RL) during the treatment phase. This machine learning model utilizes patient information (e.g. medical history and demographics, and disease severity), received disease information and estimated health status from previous health assessment models, previously generated target treatment area grid (treat vs not treat) from the classifier, and current ultrasound therapy parameters. The data is merged into a single dataset, and preprocessed (e.g. data cleaning, reduction, transformation and/or augmentation). The model is built and new therapy parameters are generated. A reward is provided based on the therapy outcomes and disease progression (e.g. positive weight if there's an improvement in the condition or negative if there's none).

In an embodiment, the reinforcement learning (RL) elements include state space, action space, and reward. The state space is the patient-related information obtained that describes patient characteristics and current condition (e.g. disease severity and state). Further, the action space is a set of treatment parameters to be modified/optimized to maximize reward (e.g., improved LVEF). The treatment parameters include but are not limited to, ultrasound-based pulsing frequency (e.g., shockwave pulsing frequency), number of treatment spots, duration of treatment, tissue depth, etc. Furthermore, the reward is provided for improved health biomarkers or clinical indicators (e.g., for cardiac application improved LVEF). The environment documents disease progression parameters over time intervals through the supervised regression and classification models used for health assessment. This allows for the sequential decision-making model to be established and optimized based on policy evaluation.

The presently disclosed device can be configured to harness non-thermal properties of ultrasound therapy to generate stem cell differentiation, angiogenesis (including but not limited to postnatal vasculogenesis), and anti-inflammatory effects, as a treatment for a plurality of diseases including but not limited to ischemia and/or fibrosis. Said non-thermal properties may be achieved through increased pressure and/or amplitude to generate microstreaming (whereby increased fluid movements can promote endothelial shear stress), jetting (whereby vascular permeability can be increased), bubble expansion and/or compression (whereby vascular permeability can be increased).

Specifically, the ultrasound therapy device can be configured to harness thermal effects of ultrasound through increased pulse length and/or power applied by means of the ultrasound transducer unit such that local tissue temperature, which may lead to liquefactive necrosis, can be increased.

Additionally, the ultrasound device can be configured to harness molecular effects. Said molecular effects may include but are not limited to the upregulation of angiogenic factors, increased nitric oxide synthase activity, anti-inflammatory properties, increased differentiation of myocytes, endothelial cells, and/or vascular smooth muscle cells.

The presently disclosed device can be used to apply ultrasound to liquefy blood clots, either independently or in combination with bubbles and anti-clotting agents, possibly being used to restore blood flow to regions.

The presently disclosed device may also be focused on harnessing non-thermal properties of ultrasound therapy to generate stem cell differentiation, angiogenesis, and anti-inflammatory effects, as a treatment for a plurality of diseases including but not limited to ischemia and/or fibrosis.

In some examples, the presently disclosed device is focused on harnessing non-thermal properties of ultrasound therapy to generate anti-inflammatory effects to inhibit fibroblast proliferation.

The presently disclosed device may be focused on harnessing non-thermal properties of ultrasound therapy to generate stem cell differentiation, angiogenesis, and anti-inflammatory effects, as a treatment for a plurality of diseases including but not limited to pulmonary fibrosis, chronic obstructive pulmonary disease (COPD), respiratory syndromes and/or pulmonary embolism.

In an embodiment, the presently disclosed device is focused on harnessing thermal properties of ultrasound therapy to target and destroy tumorous cells.

In an embodiment, the presently disclosed device is focused on harnessing ultrasound therapy to target and destroy thrombus in a plurality of body regions of the user including but not limited to the lower limb region.

In an embodiment, the presently disclosed device is used to identify and/or treat deep vein thrombosis.

In some embodiments, the same transducers can be used as sensor. In some embodiments these transducers and sensors can operate in different ranges while in other embodiments, they can operate in overlapping ranges. For example, the ultrasound transducer units 114 can act as ultrasound transducer units and ultrasound sensors in some embodiments. Alternatively, the ultrasound transducers units can act as sensor units in some embodiments, and other sensor units will be used that are more aptly tuned to desired frequencies.

### Machine Learning Model

A machine learning model underlying a machine learning system can be stored in a memory of device and/or the handheld computing device. The machine learning model can be trained within an adaptive clinical setting. The machine learning system can be integrated with the handheld computing device. Furthermore, the machine learning system can be executed remotely from a secondary handheld computing device. Additionally, the machine learning system can propose a recommended treatment, which can be accessed by the patient's clinician. The clinician can confirm the one or more recommended therapies including but not limited to the location, intensity, and/or frequency of the therapy.

In an embodiment, the machine learning system generates personalized ultrasound therapy for the user. For each user (patient), the machine learning system can measure the progression of the disease by comparing disease severity data in the period “A” with the disease severity data in the period “B”. Based on a dataset that includes all patients and/or all patients from multiple periods, the machine learning system can train a machine learning model to correlate x-variables (patient data and ultrasound therapy specifications, etc.) with the y-variables (disease progression). Based on the trained machine learning model, ultrasound therapy specifications (while holding other x-variables such as patient characteristics constant) are adjusted to minimize predicted disease progression (or in other words, maximize the efficacy of treatment). The machine learning system may include decision tree-based machine learning models, artificial neural networks, convolutional neural networks, logistic regression, naive Bayes, nearest neighbor, support vector machines, boosted tree learning methods, and/or generative neural networks.

The presently disclosed device may be configured to receive bio-sample data of the user. In operation, the user can receive his/her bio-sample from salivary glands in a form of saliva. Then the user can place the bio-sample on a reactant material having one or more reactant properties pertaining to chemical information of the user's body. Thereafter, the user can capture an image of the reactant material upon placing the bio-sample to obtain the bio-sample data. The device can then be configured to process the bio-sample data which acts as one of the decision points for generating personalized ultrasound therapy. In an alternative embodiment, the device is configured to generate personalized ultrasound therapy based on the medicine data of the user.

The device can be configured to utilize a machine learning model underlying a machine learning system stored in the memory of the user's handheld computing device. The machine learning model can for example be trained by an adaptive clinical setting. Furthermore, the machine learning system can be integrated with the handheld computing device. Additionally, the handheld computing device can also collect information about the user's health condition through a questionnaire and/or other patient health database.

A second handheld computing device may be connected to the ultrasound therapy device, enabling remote control of the ultrasound therapy device. The machine learning system can be executed remotely from a secondary handheld computing device. Furthermore, an external computing device, such as the second handheld computing device can receive data representing ultrasonic sensor data, which is recorded as a video file.

In an embodiment the device may include a machine learning model such as a large language model (LLM), to facilitate interpretation and interaction with human language. For example, the user may record their symptoms and said LLM can interpret the description of symptoms to analyze the severity of said symptoms and/or recommend next steps for treatment, trigger an alert to a third party and/or facilitate a longer conversation with the user to elucidate the state of health of that user.

### Ultrasound

Loosely organized structures, such as a thrombus or an atheroma, lacking the normal collagen and elastin fiber support, can be destroyed easily by ultrasound, but vascular walls contain a thick collagen and elastin matrix, so they tolerate ultrasound of higher intensity and lower frequency. These features are the foundation of sonothrombolysis. The present disclosure describes deploying ultrasound treatment, wherein the user receives the ultrasound therapy instructions which include disease type that needs to be treated, e.g. ischemic disease and/or organ muscle stiffness. These instructions may be predefined rules/instructions based on the user's characteristics, e.g., body size, age, type, and/or severity of organ disease—e.g., in ischemic organ disease one may want to achieve angiogenic effects that may be achieved by using ultrasound therapy parameters set at a frequency of 1/1875 MHz; 15/25 mW/cm2 spatial average temporal average (SATA); 20% duty cycle; 20 min/day).

In another example, one may want to achieve anti-inflammatory effects using parameters set at or close to a frequency of 1.5/3 MHz; 30/200 mW/cm2 SATA; 20% duty cycle; 15/20 min/day). In a further example, one may want to achieve anti-degenerative effects using parameters set at or close to a frequency of 1 MHz; 50/110 mW/cm2 SATA; 20%/50% duty cycle; 10/15 min/day). In a further example, one may want to achieve regenerative effects using parameters set at or close to a frequency of 1.5/1.6 MHz; 30/50/90 mW/cm2 SATA; 20% duty cycle; 20 min/day). In a further example, one may want to achieve differentiation effects using parameters set at or close to a frequency of 1.5/1.6 MHz; 30/50/90 mW/cm2 SATA; 20% duty cycle; 20 min/day).

The present disclosure also describes the semi-automated method of deploying ultrasound treatment. Furthermore, the present disclosure describes an automated method of deploying ultrasound treatment. For the first time usage of the automatic calibration of the wearable device to map the location and size of the user's organ, the user has to perform various steps. Additionally, for the first-time usage of manual calibration of the wearable device to map the location and size of the user's organ, the user has to perform various steps.

### Positioning Mechanism

The presently disclosed device is preferably configured such that the ultrasound transducer unit and/or the physiological sensor(s) can move controllably by means of a positioning mechanism, such that the ultrasound transducer unit, e.g. with an ultrasound sensor, can move across different regions of the user's organ.

The positioning mechanism may comprise guiding channels, defining a predefined movement path, for guiding the movement of the one or more ultrasound transducer units. One advantage thereof is that the channels allow the ultrasound transducer unit to move via an automated controller that searches for the optimum signal through the movement path.

In an embodiment, the positioning mechanism can comprise a motor for engaging with the ultrasound transducer unit, and possibly a pulley engaging with the motor. In a preferred embodiment, the device can be configured such that the motor and the pulley move controllably to position the ultrasound transducer unit.

In an embodiment, the positioning mechanism comprises a micro linear actuator, which may comprise a driving side and a driven side for engaging with a motor from the driving side, and for engaging with the ultrasound transducer unit from the driven side. Additionally, the positioning mechanism may comprise a spring-based mechanism for engaging with the ultrasound transducer unit.

In an embodiment, the wearable device is configured such that the positioning mechanism provides Pulse Width Modulation (PWM) control of the ultrasound transducer unit. An average delivered power to the device can be controlled via PWM. PWM can drive the motor in on and off modes, which can be acquired by a micro-linear actuator.

### Ultrasound Therapy

The device can be configured to execute a plurality of events. The wearable device can for example be configured to (utilizing the physiological sensor(s)) scan the user's organ, identify one or more regions of the user's organ, and possibly detect one or more organ diseases. The wearable device may further be configured to detect a plurality of physiological properties of a cardiovascular and tissue to optimize ultrasound therapy. Hence, the wearable device can be configured to determine an effective ultrasound therapy for the user in one or more regions of the user's organ region based on physiological properties and/or demographic characteristics. The wearable device can for example be configured to move the ultrasound transducer unit to the one or more identified regions and execute the ultrasound therapy in said one or more regions.

In an embodiment, the presently disclosed device is configured to analyze a health function of the organ of the user and to determine a location on the chest of the user where the ultrasound transducer unit is positioned based on physiological sensor data acquired minimally invasively.

As described herein, the device can be configured to create a map of the user's organ, wherein the map is stored in the memory and later be used to more efficiently position the physiological sensor(s), and/or the ultrasound transducer unit.

In an embodiment, the wearable device is configured to scan the organ by moving the ultrasound transducer unit, e.g. comprising the physiological sensor(s), across different regions of the user's organ and collect data therefrom.

In an embodiment, the wearable device is configured to compare the organ health of the user in the one or more regions to determine the efficacy of the ultrasound therapy over time. The wearable device can then be configured to update the ultrasound therapy based on the observed efficacy of the ultrasound therapy over time.

In an embodiment, the wearable device is configured to create a map of the user's organ, wherein the map is stored, e.g. in a memory of the device.

In an embodiment, the ultrasound transducer unit is configured to harness non-thermal properties of ultrasound therapy.

In an embodiment, the wearable device is configured to collect information on the user's health condition through a questionnaire and/or patient health database.

The present disclosure relates to the existing manual method of deploying ultrasound treatment. For example, the user can receive the ultrasound therapy information, comprising disease type that needs to be treated, e.g. ischemic disease and/or organ muscle stiffness. Then the user can position the ultrasound generator or the hereby disclosed device to the therapy region (therapy region can be approximated from the user's body size and demographics. E.g., if it's a small person, the user may be given a small wearable structure (e.g. a vest) to hold the wearable device in place) based on ultrasound therapy instructions. A wearable structure and/or device may include any device that can be worn by a user over longer periods of time, can be placed on a user's organ region either by the user themselves and/or able to be held in place by a user for a period of time lasting more than 10 seconds. Lastly, the user can apply (administer) the ultrasound therapy based on the instructions. These instructions may be predefined rules/instructions based on the user's characteristics, e.g., body size, age, type, and/or severity of organ disease. In a further embodiment, the user can manually adjust the pressure of the ultrasound transducer unit to the skin of the user to optimize contact with the user's skin for optimal ultrasound penetration. Said pressure adjustment can be performed using a pressure adjustment knob which moves the ultrasound transducer unit up and down. A pressure adjustment knob may in some cases be understood to be similarly constructed to a coarse adjustment knob of conventional microscopes that have been commonly used in clinical research.

The present disclosure further relates to a device configured to provide a semi-automated ultrasound treatment. Firstly, the user can receive ultrasound therapy information such as disease type that needs to be treated and therapy region, e.g. ischemic disease and/or organ muscle stiffness. The user can position the device to the therapy region using previously mapped out organ regions from the first-time use calibration process. Then the device can provide the ultrasonic data and/or electronic stethoscope data (and/or other minimally invasive physiological health data of the user) from the therapy region to the user. The user can therefore analyze the severity of the disease. The risk analysis may be assessed by a risk assessment machine learning model and/or measurement of physiological health function such as lung capacity measurement and/or a patient self-reported questionnaire, etc.). Then the user can identify the ultrasound therapy parameters such as intensity, duration, and/or pulsation frequency in one or more regions based on the user's characteristics, e.g., body size, age, type, and/or severity of organ disease (organ muscle stiffness may require an ultrasound therapy to relax the muscle and an ischemic disease may require an ultrasound therapy to get rid of plaque). Lastly, the user can administer the ultrasound therapy based on the identified therapy needs.

The present disclosure further relates to a device configured to provide an automated ultrasound treatment. The user can have access to the sensor data, such as ultrasonic data and/or electronic stethoscope data (and/or other minimally invasive physiological health data of the user) from a plurality of regions of the user. The user can therefore identify the one or more diseases and can analyze the severity of said one or more diseases in one or more regions. This can be done by employing a disease type and severity machine learning model, e.g., classification model trained on gold standards such as calcification index, plaque buildup in coronary arteries, measurement of physiological health function such as lung capacity, a patient self-reported health outcome/wellbeing questionnaire, etc. Then the user can identify the ultrasound therapy parameters (intensity, duration, and/or pulsation frequency) in one or more regions based on the user's characteristics, e.g. body size, age, type, and/or severity of organ disease (organ muscle stiffness may require an ultrasound therapy to relax the muscle and an ischemic disease may require an ultrasound therapy to shoot away plaque). Lastly, the user can administer the ultrasound therapy based on identified therapy needs.

The presently disclosed device can be configured to harness non-thermal properties of ultrasound therapy to generate stem cell differentiation, angiogenesis, and anti-inflammatory effects, as a treatment for a plurality of diseases including but not limited to ischemic organ disease and/or fibrosis. Said non-thermal properties may be achieved through increased pressure and/or amplitude to generate microstreaming (whereby increased fluid movements can promote endothelial shear stress), jetting (whereby vascular permeability can be increased), bubble expansion and/or compression (whereby vascular permeability can be increased).

Specifically, the wearable device can be configured to harness thermal effects of ultrasound through increased pulse length and/or power applied by means of the ultrasound transducer unit such that local tissue temperature, which may lead to liquefactive necrosis, can be increased.

Additionally, the wearable device can be configured to harness molecular effects. Said molecular effects may include but are not limited to the upregulation of angiogenic factors, increased nitric oxide synthase activity, anti-inflammatory properties, increased differentiation of myocytes, endothelial cells, and/or vascular smooth muscle cells.

The pulsation frequency and strength of the ultrasound transducer may be aimed at suppressing hypertrophic cardiomyopathy and/or myocardial interstitial fibrosis.

The ultrasound therapy may be aimed at enabling physiological health pacing. Alternatively, the presently disclosed device may be used to minimally invasively reduce hypertension by affecting the nerves that control blood pressure. Furthermore, low-intensity ultrasound pulsations may be used to create anti-inflammatory effects. Ultrasound pulsations may also be configured to generate anti-inflammatory effects to target systemic microvascular inflammation.

Low-intensity ultrasound pulsations may be used to enhance angiogenesis to reduce left ventricular dysfunction. Additionally, low-intensity ultrasound pulsations may be used to enhance angiogenesis to ameliorate myocardial infarction.

The presently disclosed device can be used to apply ultrasound to liquefy blood clots, either independently or in combination with bubbles and anti-clotting agents, possibly being used to restore blood flow to regions of the brain affected by stroke and/or treating arterial thrombosis and/or deep vein thrombosis.

Furthermore, the presently disclosed device may be focused on increasing myocardial blood flow in ischemic myocardium and endothelial cells. Ultrasound has direct effects on tissue that are cardioprotective which may arise from increased tissue blood flow induced by ultrasound and/or metabolites released from endothelial cells which may offer cardio protection by increasing blood flow.

The presently disclosed device may also be focused on harnessing non-thermal properties of ultrasound therapy to generate stem cell differentiation, angiogenesis, and anti-inflammatory effects, as a treatment for a plurality of diseases including but not limited to ischemic disease and/or fibrosis.

In some examples, the presently disclosed device is focused on harnessing non-thermal properties of ultrasound therapy to generate anti-inflammatory effects to inhibit fibroblast proliferation.

The presently disclosed device may be focused on harnessing non-thermal properties of ultrasound therapy to generate stem cell differentiation, angiogenesis, and anti-inflammatory effects, as a treatment for a plurality of diseases including but not limited to pulmonary fibrosis, chronic obstructive pulmonary disease (COPD), respiratory syndromes and/or pulmonary embolism.

In an embodiment, the presently disclosed device is focused on harnessing thermal properties of ultrasound therapy to target and destroy tumorous cells.

In an embodiment, the presently disclosed device is focused on harnessing ultrasound therapy to target and destroy thrombus in a plurality of body regions of the user including but not limited to the lower limb region.

In an embodiment, the presently disclosed device is used to identify and/or treat deep vein thrombosis.

The techniques described herein relate to an ultrasound system, wherein the ultrasound therapy information includes location data, frequency data, spatial average, temporal average data, duty cycle data, and therapy duration data.

The present disclosure presents shockwaves as a type of ultrasound therapy. Shockwave parameters included as part of the ultrasound therapy information comprise but are not limited to number of spots to treat, number of shocks per spot, positive peak duration and amplitude, negative tensile peak duration and amplitude, pulse duration and frequency of pulse repetition, pulse train frequency, total therapeutical session duration and treatment frequency occurrence and duration.

The present disclosure describes the Pulse Train Theory behind the application of Shockwave Treatment.

Unless otherwise defined, all terms (including technical and scientific terms) used in this disclosure have the same meaning as commonly understood by one of ordinary skill in the art to which this disclosure belongs. In this specification, “mobile phone” and “smartphone” are interchangeable as are “tablet” and “smart tablet.” It is to be understood that the phrases or terms employed herein are for description and not of limitation.

As will be appreciated by one of the skills in the art, the present disclosure may be embodied as a device, system, and method, or computer program product. Further, the present invention may take the form of a computer program product on a computer-readable storage medium having computer-usable program code embodied in the medium. The present systems and methods have been described above with reference to specific examples. However, other embodiments and examples than the above description is equally possible within the scope of the present invention. The scope of the disclosure may only be limited by the appended patent claims. Repeated use of “in some embodiments” or “in an embodiment” is not intended to refer to the same embodiments unless required by the context.

Even though modifications and changes may be suggested by the persons skilled in the art, it is the intention of the inventors and applicants to embody within the patent warranted heron all the changes and modifications as reasonably and properly come within the scope of the contribution the inventors and applicants to the art. The scope of the embodiments of the present invention is ascertained with the claims to be submitted at the time of filing the complete specification. Method steps can be performed in any order unless required otherwise by the context. In the specification and claims, a feature mentioned in the singular (e.g., using “a” or “an”) will be deemed to have an “at least one” or plural construction except where the context indicates such construction unworkable.

A person of skill in the art will also recognize that the embodiments discussed herein are reconfigurable and within the intended scope. For example, the dependent claims from one independent claim or dependent claim can be similarly made to depend from a different independent claim and/or dependent claim, unless prohibited by the context. In addition, as would be appreciated by a person of skill in the art, certain features or elements of a claim can be mixed and matched with other features or elements, even if not presented together at the time of filing.

Similarly, as would be appreciated by a person of skill in the art, data, outputs and readings from different described sensors, user inputs and other sources can be used together, even if not presented together at the time of filing. The term “and/or” in a list means all list items present, some list items present, or one of the list items present, unless such construction is limited by the context.

Positional and directional terms described in this specification may be understood to be different than shown or described and should not limit the variations of embodiments possible from the claimed features that a person of ordinary skill in the art would understand from the specification, figures and claims. “Acceptable,” unless the context dictates otherwise, refers to meeting a clinical threshold or such action would achieve a positive clinical outcome given the situation. “Threshold,” unless the context dictates otherwise, refers to equipment or sensor operation according to best practice or achieving such a position would achieve a positive clinical outcome given the situation. “Surrounding” means around the ultrasound therapy area. “Data” as used herein can be singular or plural.

1. A device for generating ultrasound treatment to a target therapy area of a user, the device comprising:
a wearable housing including a skin-facing side;
an array of ultrasound transducer units on the skin-facing side;
an energy source; and
a processor for processing instructions, the instructions comprising;
loading transducer parameters for a therapeutic application, the transducer parameters containing at least one of frequency, intensity, treatment zone area, and duration;
triggering the array of ultrasound transducer units to emit test waves according to a transmission quality test routine;
determining if a backscatter or a reflection of the test waves passes the transmission quality test;
initiating an ultrasound therapy treatment process when the current conditions pass the transmission quality test; the ultrasound treatment process comprising
generating an ultrasound therapy by the array of ultrasound transducer units.

2. The device according to claim 1, wherein the ultrasound treatment process further comprises, before generating the ultrasound therapy, setting ultrasound therapy parameters at least partly based on real-time or near real-time data from one or more physiological sensors; the therapy parameters including one or more of pulsing frequency, pulse train frequency, number of treated spots, duration, treatment zone area.

3. The device according to claim 2, wherein the ultrasound treatment process further comprises, after generating the ultrasound therapy, adjusting the ultrasound therapy parameters during the treatment process if needed at least partly based on updated real-time or near real-time data from one or more physiological sensors.
wherein the instructions further comprise receiving a health assessment of the user based on received health status data.

4. The device according to claim 2 or 3, wherein the ultrasound therapy parameters comprise one or more of pulsing frequency, pulse train frequency, number of treated spots, duration, and treatment zone are.

5. The device according to any of the preceding claims, further comprising a communication system.

6. The device according to claim 5, wherein the instructions further comprise receiving a health assessment of the user based on received health status data.

7. The device according to claim 5 or 6, wherein the device is further connected to a device comprising a display screen further comprising a user interface to receive input information about the target therapy area, wherein the input information comprises size and shape of the target therapy area.

8. The device according to any of claims 5-7, further comprising a user-friendly interface allowing the user to interact with the device, monitor therapy progress, adjust settings, and receive alerts or updates.

9. The device according to any of claims 5-8, wherein the communication system is configured to relay therapy data and receive remote operational commands.

10. The device according to any of the preceding claims, further comprising one or more physiological sensors, wherein the one or more physiological sensors are selected from a group consisting of one or more minimally invasive sensors including but not limited to an electrocardiography (ECG) sensor, photoplethysmography (PPG) sensor, seismocardiography (SCG) sensor, ballistocardiography (BCG) sensor, phonocardiography (PCG) sensor, ultrasonic sensor, temperature sensor, blood pressure sensor, bioimpedance sensor, electromyography (EMG), blood biomarker, pacemaker, glucose sensor, cochlear implants, implantable defibrillators, electroencephalogram (EEG), pulse oximeters, deep brain stimulators, retina implants, intracardiac pressure monitoring sensors and/or a piezoelectric sensor.

11. The device according to any of the preceding claims, further comprising a circuitry to automatically shut off the device upon detection of one or more of excessive heat, pain, and an indicator of potential harm.

12. The device according to any of the preceding claims, further comprising an emergency stop button to allow the user to terminate the ultrasound therapy as and when required.

13. The device according to any of the preceding claims, further comprising wherein the transmission quality test is used to determine the quality of the ultrasound transmission or the proximity of the transducers to the skin.

14. The device according to any of the preceding claims, wherein the target therapy area comprises at least one of a thoracic region, head region, kidney region, a lung region, a lower limb region, a neck region, a shoulder region, an upper limb region, an abdominal region, and a back region of the user.

15. The device according to any of the preceding claims, wherein the array of ultrasound transducer units is configured to operate directly through the skin of the user near the target therapy area or indirectly through one or more of an impedance matching material, gel, and fluid near the target therapy area.

16. The device according to any of the preceding claims, wherein the instructions further include determining whether to not utilize, partially utilize, or fully utilize each of the array of transducer units and activating a selection of the array of transducer units for beam steering of the ultrasound and shifting focal points of the ultrasound to a desired location of the target therapy area.

17. The device according to any of the preceding claims, wherein the instructions include adjusting a phase and a magnitude of each of the ultrasound transducers in the array of ultrasound transducers.

18. The device according to any of the preceding claims, further comprising a minimally invasive sensor, and the instructions include acquiring through the minimally invasive sensor one or more of: cardiac function information, neuronal function information, pulmonary function information, renal function information, peripheral arterial function, and deep vein thrombosis (DVT) function information of the user.

19. The device according to any of the preceding claims, further comprising a vibration sensor on a side of the wearable housing facing away from the skin-facing side, wherein the instructions include detecting and removing of vibration disturbances sensed by the vibration sensor with receiving and sending signals.

20. The device according to any of the preceding claims, wherein the instructions include promoting one or more of angiogenesis, reducing inflammation, improving fibrosis, tubular injury, neovascularizing, and ameliorating inflammatory processes in the target therapy area.

21. The device according to any of the preceding claims, further comprising an inertial measurement unit (IMU) sensor, and the instructions include determining with the IMU sensor at least one of whether the ultrasound therapy device is correctly positioned on the skin and whether the user is moving.

22. The device according to any of the preceding claims, wherein the housing is designed to contour to one or more of the user's chest, head, thorax and lower-limb region.

23. The device according to any of the preceding claims, wherein the instructions include comparing one or more of pulmonary health, renal health, peripheral artery health, neuronal health, cardiac health and/or deep vein thrombosis health of the user in one or more of the target therapy areas over time to determine the efficacy of the ultrasound therapy.

24. The device according to claim 23, wherein the processor is configured to update the ultrasound therapy based on the determined efficacy of the ultrasound therapy over time.

25. The device according to any of the preceding claims, wherein the wearable housing is rigid or semi-rigid.

26. The device according to any of the preceding claims, wherein the wearable housing comprises an adjustable strapping mechanism for secure attachment to the user's body.

27. The device according to any of the preceding claims, wherein the wearable housing comprises an adhesive patch.

28. The device according to any of the preceding claims, wherein the processor executes instructions to initiate shockwave therapy with an energy level of less than 0.38 mJ/mm².

29. The device according to any of the preceding claims, wherein the grouping of ultrasound transducers is arranged in an array, wherein the array configuration comprises a specific arrangement of at least one of the following patterns:
a linear array for focused ultrasound therapy;
a curved array arranged in a concave shape to follow the contour of the body in irregular surfaces; and
an annular array following an annular pattern.

30. The device according to any of the preceding claims, further configured to provide an extracorporeal shockwave treatment in parallel with chemotherapy for cancer treatment to minimize the risk of cancer therapy-induced cardiomyopathies and/or congestive heart failure.

31. The device according to any of the preceding claims, further configured to provide an extracorporeal shockwave treatment in parallel with chemotherapy for cancer treatment wherein the treatment is administered prior to each chemotherapy session during the cancer treatment regimen.

32. The device according to claim 31, wherein the extracorporeal shockwave treatment is synchronized with chemotherapy sessions using a smart algorithm that dynamically adjusts the timing and intensity of shockwaves based on the specific amount and type of chemotherapy drugs administered.

33. The device according to any of the preceding claims, further comprising a control unit and a user interface allowing healthcare professionals to program and customize the initial shockwave treatment parameters based on individual patient profiles.

34. The device according to any of the preceding claims, wherein extracorporeal shockwave treatment is administered using adjustable shockwave parameters including one or more of frequency, intensity, and duration, tailored to individual patient needs and clinical presentations.

35. A method for generating ultrasound treatment to a target therapy area of a user, the method comprising the steps of:
providing the user with an ultrasound therapy device containing a housing with ultrasound transducers on a skin-facing side of the housing, an impedance-matching material, gel or fluid, and a user interface;
applying onto the user the impedance matching material, gel, or fluid at or near a target therapy area;
holding by the user or fastening within a wearable structure the ultrasound therapy device against the impedance matching material, gel, or fluid of the user at or near the target therapy area;
sensing, by the ultrasound therapy device, and receiving, by a user interface, information regarding at least some of a location of the target therapy area, a location of the ultrasound therapy device relative to the target therapy area, an ultrasound transmission quality of the impedance matching material, gel or fluid and skin contact, a type of ultrasound therapy to perform, and a health status of the user;
activating the ultrasound therapy by the user; and
performing the ultrasound therapy on the user based on the information.

36. The method according to claim 35, further comprising the steps of:
mapping out a sub-skin layout of the target therapy area using the ultrasound transducers or other sensors on the device;
labeling the features identified in the sub-skin layout; and
performing the ultrasound therapy on a portion of the sub-skin layout that matches a pre-determined label.

37. The method according to claim 35 or 36, further comprising the step of:
focusing a laser from the ultrasound therapy device or another device below the user's skin near the target therapy area to create an ultrasound emanating from a focal point of the laser by means of laser energy absorbance and the resulting rapid thermal expansion which generates ultrasound waves detected by the ultrasound transducers in the device, utilized for therapy guidance and sub-skin layout mapping allowing for precise targeting and monitoring of therapy area.

38. The method according to any of claims 35-37, further comprising the step of terminating the ultrasound therapy upon depression, actuation, or toggling of an emergency stopper on or near the ultrasound therapy device.

39. The method according to any of claims 35-38, wherein the target therapy area comprises at least one of a thoracic region, head region, kidney region, a lung region, a lower limb region, a neck region, a shoulder region, an upper limb region, an abdominal region, and a back region of the user.

40. The method according to any of claims 35-39, wherein the ultrasound therapy is performed to initiate shockwave therapy with an energy level of less than 0.38 mJ/mm² to induce angiogenesis in a cardiac tissue of the user.

41. The method according to any of claims 35-40, wherein the ultrasound therapy is administered in a population with above average risk factors for developing a stroke.

42. The method according to any of claims 35-41, wherein the ultrasound therapy is performed using a shockwave therapy parameter with an energy level of less than 0.04 mJ/cm² prior to administering a chemotherapy drug to the user.

43. The method according to any of claims 35-42, wherein the ultrasound therapy is performed during a drug-coated balloon interventional procedure.

44. The method according to any of claims 35-43, further comprising the steps of receiving information about the ultrasound therapy and controlling the ultrasound therapy through the user interface.

45. The method according to any of claims 35-44, further comprising a step of implementing gating procedures.

46. A system for shockwave therapy utilizing an ultrasound system, comprising:
an ultrasound therapy module configured to generate ultrasound therapy information, wherein the ultrasound therapy information includes location data, frequency data, spatial average, temporal average data, duty cycle data, and therapy duration data;
a shockwave generation module configured to generate shockwaves as a type of shockwave therapy, wherein a shockwave is defined as a single pulse with a wide frequency range of approximately 150 kHz up to 100 MHz, a high pressure amplitude of up to 150 MPa, a low tensile wave of up to −25 MPa, a small positive pulse width lower than 1000 nanoseconds (1 μs) and a total pulse width lower than 10 μs, and a short rise time of up to a few hundred nanoseconds;
a treatment parameter adjustment module configured to adjust shockwave parameters as part of the ultrasound therapy information, including the number of spots to treat, the number of shocks per spot, positive peak duration and amplitude, negative tensile peak duration and amplitude, pulse duration, frequency of pulse repetition, pulse train frequency, total therapeutic session duration, and treatment frequency occurrence and duration;
a pulse train control module configured to control the application of multiple shocks into a damaged area by dividing the target area into spots and applying multiple shocks to each spot, wherein a group of shocks applied on the same spot is referred to as the train of pulses, and the time between shocks applied on the same spot determines the final pulse repetition frequency of the treatment, while the time between consecutive train of pulses for the treatment of two consecutive target spots determines the pulse train frequency of the therapy;
a therapeutic outcome monitoring module configured to monitor and analyze the therapeutic outcomes of shockwave therapy based on the adjusted shockwave parameters; and
a user interface module configured to provide a graphical user interface for adjusting shockwave parameters and monitoring therapeutic outcomes.

47. A non-invasive ultrasound therapy system for training a machine learning model to optimize ultrasound therapy parameters, the system comprising:
a computing device or cloud system connected to receive post-ultrasound therapy health outcome data comprising changes in one or more of a cardiac function, quality of life and angiogenesis, patient information data comprising demographic and medical history data of patients and ultrasound therapy parameter data comprising one or more of pulsing frequency, pulse train frequency, number of treated spots, duration and treatment zone area from a patient population comprising of suspected or confirmed cardiovascular disease patients from multiple clinical study sites, wherein the computing device or cloud system comprises a processor to execute a plurality of instructions comprising:
separately match the post-ultrasound therapy health outcome patient data with the patient information data and ultrasound therapy parameter data;
generate features from at least one portion of the patient information or ultrasound therapy parameter data using manual methods or unsupervised machine learning methods; and
train a machine learning model based on the generated features to optimize ultrasound therapy parameters using unsupervised or supervised machine learning methods, wherein the ultrasound therapy parameters are optimized to increase the likelihood of achieving a shockwave therapeutic effect comprising angiogenesis and neovascularization.

48. The system according to claim 47, further comprising storing the machine learning model in a format compatible to be implemented on a portable computing device.
